SEQUENCE LISTING (67 sequences)

SEQ ID NO: 1 - protein, 323 aa, Homo sapiens
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 2 - protein, 297 aa, Homo sapiens
HTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 3 - protein, 26 aa, Homo sapiens
MARGPGLAPPPLRLPLLLLVLAAVTG

SEQ ID NO: 4 - protein, 248 aa, Homo sapiens
HTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLT

SEQ ID NO: 5 - protein, 47 aa, Homo sapiens
HTAAQDNCTCPTNKMTVCSPDGPGGRCQCRALGSGMAVDCSTLTSKC

SEQ ID NO: 6 - protein, 73 aa, Homo sapiens
LLLKARMSAPKNARTLVRPSEHALVDNDGLYDPDCDPEGRFKARQCNQTSVCWCVNSVGVRRTDKGDLSLRCD

SEQ ID NO: 7 - protein, 128 aa, Homo sapiens
ELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLT

SEQ ID NO: 8 - protein, 23 aa, Homo sapiens
AGLIAVIVVVVVALVAGMAVLVI

SEQ ID NO: 9 - protein, 26 aa, Homo sapiens
TNRRKSGKYKKVEIKELGELRKEPSL

SEQ ID NO: 10 - protein, 450 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 13 - protein, 8 aa, synthetic construct
GYTFTNYG

SEQ ID NO: 14 - protein, 8 aa, synthetic construct
INTYTGEP

SEQ ID NO: 15 - protein, 14 aa, synthetic construct
ARGGFGSSYWYFDV

SEQ ID NO: 16 - protein, 25 aa, synthetic construct
QVQLQQSGSELKKPGASVKVSCKAS

SEQ ID NO: 17 - protein, 17 aa, synthetic construct
MNWVKQAPGQGLKWMGW

SEQ ID NO: 18 - protein, 38 aa, synthetic construct
TYTDDFKGRFAFSLDTSVSTAYLQISSLKADDTAVYFC

SEQ ID NO: 19 - protein, 11 aa, synthetic construct
WGQGSLVTVSS

SEQ ID NO: 20 - protein, 110 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTV

SEQ ID NO: 21 - protein, 6 aa, synthetic construct
QDVSIA

SEQ ID NO: 22 - skipped

SEQ ID NO: 23 - protein, 9 aa, synthetic construct
QQHYITPLT

SEQ ID NO: 24 - protein, 26 aa, synthetic construct
DIQLTQSPSSLSASVGDRVSITCKAS

SEQ ID NO: 25 - protein, 17 aa, synthetic construct
VAWYQQKPGKAPKLLIY

SEQ ID NO: 26 - protein, 36 aa, synthetic construct
YRYTGVPDRFSGSGSGTDFTLTISSLQPEDFAVYYC

SEQ ID NO: 27 - protein, 13 aa, synthetic construct
FGAGTKVEIKRTV

SEQ ID NO: 28 - protein, 450 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 214 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 121 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 31 - protein, 8 aa, synthetic construct
GYTFTTAG

SEQ ID NO: 32 - protein, 8 aa, synthetic construct
INTHSGVP

SEQ ID NO: 33 - protein, 14 aa, synthetic construct
ARSGFGSSYWYFDV

SEQ ID NO: 34 - protein, 25 aa, synthetic construct [sequence omitted]

SEQ ID NO: 35 - protein, 17 aa, synthetic construct
MQWVRQAPGQGLEWMGW

SEQ ID NO: 36 - protein, 38 aa, synthetic construct
KYAEDFKGRVTISADTSTSTAYLQLSSLKSEDTAVYYC

SEQ ID NO: 37 - protein, 11 aa, synthetic construct
WGQGTLVTVSS

SEQ ID NO: 38 - protein, 110 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTV

SEQ ID NO: 39 - protein, 6 aa, synthetic construct
QDVSTA

SEQ ID NO: 40 - skipped

SEQ ID NO: 41 - protein, 9 aa, synthetic construct
QQHYITPLT

SEQ ID NO: 42 - protein, 26 aa, synthetic construct [sequence omitted]

SEQ ID NO: 43 - protein, 17 aa, synthetic construct
VAWYQQKPGKAPKLLIY

SEQ ID NO: 44 - protein, 36 aa, synthetic construct
YRYTGVPSRFSGSGSGTDFTLTISSLQPEDFAVYYC

SEQ ID NO: 45 - protein, 13 aa, synthetic construct
FGQGTKLEIKRTV

SEQ ID NO: 46 - protein, 330 aa, synthetic construct [sequence omitted]

SEQ ID NO: 47 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 48 - protein, 12 aa, synthetic construct
EPKSCDKTHTCP

SEQ ID NO: 49 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 50 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 53 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 219 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 107 aa, synthetic construct [sequence omitted]

SEQ ID NO: 57 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 58 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 59 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 60 - protein, 106 aa, synthetic construct
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 61 - protein, 106 aa, synthetic construct
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 62 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 113 aa, synthetic construct
PCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAK

SEQ ID NO: 65 - protein, 220 aa, synthetic construct
PCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 219 aa, synthetic construct
PCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 16 aa, synthetic construct
QGRGGLDLRVRGEPLQ

(19) United States
(12) Patent Application Publication
(10) Pub. No.: US 2026/0355342 A1
(43) Pub. Date: Nov. 12, 2026

(54) TROP2-BINDING MOLECULES

(71) Applicant: Hummingbird Bioscience Pte. Ltd., Singapore (SG)

(72) Inventors: Jerome BOYD-KIRKUP, Singapore (SG); Piers INGRAM, Singapore (SG); Gary KHOO, Singapore (SG); Brendon HANSON, Singapore (SG); Dipti THAKKAR, Singapore (SG); Benjamin AYERS, Singapore (SG); Lisa OOI, Singapore (SG); Kevin Fongching CHAU, Singapore (SG); Youngjoo CHOI, Singapore (SG); Xiu Li SIM, Singapore (SG)

(21) Appl. No.: 19/704,333

(22) Filed: Jun. 10, 2026

Related U.S. Application Data
(63) Continuation of application No. PCT/SG2025/050488, filed on Jul. 18, 2025.
(60) Provisional application No. 63/673,373, filed on Jul. 19, 2024.

(51) Int. Cl. A61K 47/68; C07K 16/30

(52) U.S. Cl. CPC A61K 47/6851; A61K 47/6803; A61K 47/68037; A61K 47/6889; C07K 16/30

(57) ABSTRACT

Provided here are antigen-binding molecules that bind to TROP2, comprising (i) a TROP2-binding moiety, and (ii) at least one linker-payload moiety, wherein the antigen-binding molecule comprises (a) a DNA damage response (DDR) inhibitor moiety, and (b) a DNA topoisomerase I (TOP1) inhibitor moiety.

### CROSS-REFERENCE TO RELATED APPLICATIONS

This application claims priority to and benefit of U.S. Provisional Patent Application No. 63/673,373, filed Jul. 19, 2024, the disclosure of which is hereby incorporated herein by reference in its entirety.

### Reference to an Electronic Sequence Listing

The content of the electronic sequence listing (366592000440seqlist.xml; Size: 63,967 bytes; and Date of Creation: Jul. 16, 2025) is herein incorporated by reference in its entirety.

### TECHNICAL FIELD

The present disclosure relates to molecular biology, more specifically antibody technology. The present disclosure also relates to methods of medical treatment and prophylaxis.

### BACKGROUND

Cancers remain the leading cause of deaths worldwide. Chemotherapies have good clinical benefits, but due to their low specificity they have very significant side effects and low therapeutic indices. More targeted therapies, such as monoclonal antibody therapies, show good specificity but response rates are smaller. Antibody-drug conjugates (ADCs) are a therapeutic modality that harness an antibody's target specificity to selectively deliver cytotoxic payloads to tumors and are proving increasingly effective in the clinic.

TROP2 is a transmembrane glycoprotein encoded by the TACSTD2 gene. TROP2 is an intracellular calcium signal transducer. Under physiological conditions, TROP2 plays an essential role in embryonic development, placental tissue formation, embryo implantation, stem cell proliferation, organ development, and tight junction maintenance. TROP2 can also activate multiple pro-oncogenic signals including the MAPK/ERK pathway, the cyclin D/E pathway, and β-catenin signalling.

Although expression is observed in various normal tissues, overexpression of TROP2 is observed across many tumour types. TROP2 expression also has prognostic significance. A meta-analysis of 16 observational studies consisting of 2,569 solid tumor patients showed high TROP2 expression was significantly correlated with poor overall survival and poor disease-free survival (Zeng et al., Scientific Reports (2016) 6:33658).

Anti-TROP2 antibody drug conjugates (ADCs) are described in Parisi et al., Cancer Treatment Reviews (2023) 118:102572, and include sacituzumab govitecan (DrugBank Acc. No. DB12893; marketed as Trodelvy™) and datopotamab deruxtecan (DrugBank Acc. No. DB16410). Sacituzumab govitecan comprises the cytotoxic agent SN-38 (a topoisomerase I inhibitor) conjugated to a humanised antibody (RS7) via a hydrolysable linker. Datopotamab deruxtecan comprises the DNA topoisomerase I inhibitor DS-8201a (DXd) conjugated to a humanised anti-TROP2 antibody via a tetrapeptide-based cleavable linker. Despite showing promising overall response rates in some cancer indications, the majority of responders progress within one year.

There remains an unmet clinical need for the effective treatment of TROP2-expressing cancers.

### SUMMARY

In a first aspect, the present disclosure provides an antigen-binding molecule that binds to TROP2, comprising (i) a TROP2-binding moiety, and (ii) at least one linker-payload moiety, wherein the antigen-binding molecule comprises (a) a DNA damage response (DDR) inhibitor moiety, and (b) a DNA topoisomerase I (TOP1) inhibitor moiety.

In some embodiments, the DDR inhibitor moiety is, or comprises, a DDR inhibitor selected from: an ATR inhibitor, a PARP inhibitor, an ATM inhibitor, a WEE1 inhibitor, a CHK1/2 inhibitor, a DNA-PK inhibitor, a PLK1 inhibitor, a Polθ inhibitor, a RAD51 inhibitor, a USP inhibitor, a PKMYT1 inhibitor, or an Aurora-A inhibitor.

In some embodiments, the DDR inhibitor moiety is, or comprises, a DDR inhibitor which is an ATR inhibitor, optionally wherein the DDR inhibitor moiety is, or comprises, berzosertib.

In some embodiments, the TOP1 inhibitor moiety is, or comprises, a TOP1 inhibitor selected from: camptothecin or a derivative thereof, exatecan, exatecan mesylate (DX-8951f), N-glycyl-exatecan, SN-38, DXd (1), DXd (2), irinotecan, etirinotecan, FL118, topotecan, gimatecan, belotecan, deruxtecan, belotecan, rubitecan, lurtotecan, diflomotecan, karenitecan, silatecan, namitecan, elomotecan, DRF-1042, delimotecan, NSC606985, chimmitecan, ZBH-1205, Genz-644282, non-CPT1, indotecan, indimitecan, AZ14170132, SHR9265, Ed-04, KL610023, A1.9, ZD06519, P1003, P1021, VIP126, ZBH-01 and LMP-744.

In some embodiments, the TOP1 inhibitor moiety is, or comprises, a TOP1 inhibitor selected from: camptothecin or a derivative thereof, exatecan, exatecan mesylate (DX-8951f), N-glycyl-exatecan, SN-38, DXd (1), DXd (2), optionally wherein the TOP1 inhibitor moiety is, or comprises exatecan.

In some embodiments, the antigen-binding molecule comprises a linker-payload moiety comprising (a) a DDR inhibitor moiety, and (b) a TOP1 inhibitor moiety.

In some embodiments, the TROP2-binding moiety comprises:
(i) a heavy chain variable (VH) region incorporating the following CDRs:
   HC-CDR1 having the amino acid sequence of SEQ ID NO:13
   HC-CDR2 having the amino acid sequence of SEQ ID NO:14
   HC-CDR3 having the amino acid sequence of SEQ ID NO:15; and
(ii) a light chain variable (VL) region incorporating the following CDRs:
   LC-CDR1 having the amino acid sequence of SEQ ID NO:21
   LC-CDR2 having the amino acid sequence of SEQ ID NO:22
   LC-CDR3 having the amino acid sequence of SEQ ID NO:23.

In some embodiments, the antigen-binding moiety that binds to TROP2 comprises:
a VH region comprising an amino acid sequence having at least 70% sequence identity to the amino acid sequence of SEQ ID NO:12; and
a VL region comprising an amino acid sequence having at least 70% sequence identity to the amino acid sequence of SEQ ID NO:20.

In some embodiments, the antigen-binding moiety that binds to TROP2 comprises:
a polypeptide comprising, or consisting of, an amino acid sequence having at least 70% sequence identity to the amino acid sequence of SEQ ID NO:10; and
a polypeptide comprising, or consisting of, an amino acid sequence having at least 70% sequence identity to the amino acid sequence of SEQ ID NO:11.

The present disclosure also provides a composition comprising an antigen-binding molecule according to the present disclosure, and a pharmaceutically acceptable carrier, diluent, excipient or adjuvant.

The present disclosure also provides an antigen-binding molecule or a composition according to the present disclosure, for use in a method of medical treatment or prophylaxis, or in a method of diagnosis or prognosis.

The present disclosure also provides an antigen-binding molecule or a composition according to the present disclosure, for use in treating or preventing a cancer.

The present disclosure also provides use of an antigen-binding molecule or a composition according to the present disclosure, in the manufacture of a medicament for treating or preventing a cancer.

The present disclosure also provides a method of treating or preventing a cancer, comprising administering to a subject a therapeutically- or prophylactically-effective amount of an antigen-binding molecule or a composition according to the present disclosure.

In some embodiments, the cancer is selected from: a cancer comprising cells expressing/overexpressing TROP2, a solid tumour, metastatic cancer, unresectable cancer, hormone receptor-positive cancer, lung cancer, non-small cell lung cancer (NSCLC), non-small cell lung cancer adenocarcinoma (adNSCLC), squamous non-small cell lung cancer (sqNSCLC), breast cancer, hormone receptor-positive breast cancer, metastatic breast cancer, hormone receptor-positive metastatic breast cancer, HER2-positive breast cancer, HER2-positive metastatic breast cancer, HER2-negative breast cancer, metastatic HER2-negative breast cancer, unresectable HER2-negative breast cancer, hormone receptor-positive and HER2-negative breast cancer, metastatic hormone receptor-positive and HER2-negative breast cancer, unresectable hormone receptor-positive and HER2-negative breast cancer, triple-negative breast cancer (TNBC), metastatic triple negative breast cancer, unresectable triple-negative breast cancer, pancreatic cancer, prostate cancer, castration-resistant prostate cancer (CRPC), metastatic castration-resistant prostate cancer (mCRPC)), bladder cancer, head and neck cancer, head and neck squamous cell carcinoma (HNSCC), endometrial cancer, ovarian cancer, esophageal cancer, esophageal squamous-cell carcinoma (ESCC), cervical cancer, thyroid cancer, biliary tract cancer (BTC), urinary tract cancer, urothelial cancer, metastatic urothelial cancer, and unresectable urothelial cancer.

In some embodiments, the cancer is refractory or relapsed to treatment with a DNA damage repair inhibitor, and/or wherein the cancer is refractory or relapsed to treatment with a DNA topoisomerase I inhibitor.

The present disclosure also provides use of an antigen-binding molecule or a composition according to the present disclosure, to deplete or to increase killing of cells expressing TROP2.

The present disclosure also provides an in vitro complex, optionally isolated, comprising an antigen-binding molecule according to the present disclosure bound to TROP2.

### DESCRIPTION

The present disclosure relates to antigen-binding molecules comprising a TROP2-binding moiety and a linker-payload moiety comprising at least two different payload moieties.

The antigen-binding molecules of the present disclosure are provided with unexpected and advantageous properties relative to known anti-TROP2 antibody-drug conjugates.

DNA damage response (DDR) is a key pathway for repair, and might be an important mode of resistance to DNA damaging payloads such as TOP1 inhibitors. Synergistic anticancer effects have previously been observed through combined treatment using a DDR inhibitor and a TOP1 inhibitor (Thomas et al., Cancer Cell. (2021) 39 (4): 566-79 e7). In embodiments wherein the antigen-binding molecule of the present disclosure comprises a linker-payload moiety comprising (i) a TOP1 inhibitor moiety and (ii) a DNA damage response (DDR) inhibitor moiety, the DDR inhibitor is thought to increase the sensitivity of the cells of the cancer to the TOP1 inhibitor. Moreover, many TOP1 inhibitors and DDR inhibitors are not substrates for P-glycoprotein, and so patients treated with antigen-binding molecules comprising such payload moieties are less likely to develop P-glycoprotein-mediated resistance to such therapy.

### TROP2

TROP2 (trophoblast cell surface antigen 2) is the protein identified by UniProtKB: P09758. TROP2 is also known e.g. as tumor-associated calcium signal transducer 2 (TACSTD2), gastric antigen 733-1 (GA733-1), epithelial glycoprotein-1 (EGP-1), and membrane component chromosome 1 surface marker 1 (M1S1). Human TROP2 has the amino acid sequence of P09758 (v3, 2007 Nov. 13; SEQ ID NO:1). Human TROP2 is encoded by the human TACSTD2 gene.

Human TROP2 comprises a 26 amino acid N-terminal signal peptide (SEQ ID NO:3), followed by an extracellular domain (SEQ ID NO:4), a single-pass transmembrane domain (SEQ ID NO:8), and a cytoplasmic domain (SEQ ID NO:9) at the C-terminus. The mature form of human TROP2 is shown in SEQ ID NO:2.

The extracellular domain comprises a cysteine-rich domain (CRD) (SEQ ID NO:5), a thyroglobulin type-1 domain (TY-1) (SEQ ID NO:6), and a cysteine-poor domain (CPD) (SEQ ID NO:7). TROP2 has 4 N-linked glycosylation sites in the extracellular domain at the residues 33, 120, 168, and 208.

TROP2 monomers may form cis- or trans-dimers. At the interface of a TROP2 cis-dimer, a loop from the TY-1 domain from one monomer interacts with the CPD BC sheet of the other monomer via hydrogen bonds. The interface of a trans-dimer is formed by interaction between the CPD BC sheets of two TROP2 monomers. TROP2 cis- or trans-dimers can form tetramers. Tetrameric assembly of two cis- or trans-dimers occurs via the CRD domains.

The intracellular domain of TROP2 comprises a phosphatidylinositol 4,5-bisphosphate (PIP2)-binding motif, a conserved tyrosine site, and a serine phosphorylation site at S303. Part of TROP2 signaling is dependent on protein kinase C (PKC) and involves phosophyorylation of S303.

The structure and function of TROP2 is described e.g. in Shvartsur & Bonavida, Genes Cancer. (2015) 6 (3-4): 84-105, which is hereby incorporated by reference in its entirety.

TROP2 has been implicated in numerous intracellular signaling pathways. TROP2 transduces an intracellular calcium signal. TROP2-induced signal transduction can occur without extracellular Ca2+, suggesting a mobilization of Ca2+ from internal stores. TROP2 provides crucial signals for cells with requirements for proliferation, survival, self-renewal, and invasion. TROP2 has several ligands, including claudin-1, claudin-7, cyclin D1, IGF-1 and PKC. TROP2 has stem cell-like qualities and regulates cell growth, transformation, regeneration, and proliferation, which explains why its overexpression can lead to tumor progression. It is expressed on the surface of many stem/progenitor cells and has a role in maintaining tight junction integrity. TROP2 is also purported to be a modulator and/or an enhancer of EpCAM-induced cell signaling. TROP2 modulation of EpCAM can cause EpCAM to proliferate and migrate into liver parenchyma.

Reference herein to ‘TROP2’ generally refers to the canonical isoform of the human TROP2 (i.e. isoform 1), but also contemplates isoforms, fragments, variants (including mutants) and homologues thereof (i.e. from other species, e.g. non-human mammalian species (e.g. a non-human primate, e.g. rhesus, cynomolgous; e.g. a rodent, e.g. rat or mouse).

As used herein, a ‘fragment’, ‘variant’ or ‘homologue’ of a protein may optionally be characterised as having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98% or ≥99% amino acid sequence identity to the amino acid sequence of the reference protein (e.g. the canonical isoform of the human protein). In some embodiments fragments/variants/isoforms/homologues may be characterised by ability to perform a function performed by the reference protein.

A ‘fragment’ generally refers to a fraction of the reference protein. A ‘variant’ generally refers to a protein having an amino acid sequence comprising one or more amino acid substitutions, insertions, deletions or other modifications relative to the amino acid sequence of the reference protein, but retaining a considerable degree of sequence identity (e.g. at least 60%) to the amino acid sequence of the reference protein. An ‘isoform’ generally refers to a variant of the reference protein expressed by the same species as the species of the reference protein. A ‘homologue’ generally refers to a variant of the reference protein produced by a different species as compared to the species of the reference protein. For example, human TROP2 (P09758 v3; SEQ ID NO:1) and mouse TROP2 (UniProt: P, v1, 2003 Mar. 1) are homologues of one another. Homologues include orthologues.

A ‘fragment’ may be of any length (by number of amino acids), although may optionally be at least 20% of the length of the reference protein (that is, the protein from which the fragment is derived) and may have a maximum length of one of 50%, 75%, 80%, 85%, 90%, 91%, 92%, 93%, 94%, 95%, 96%, 97%, 98%, or 99% of the length of the reference protein. A fragment of TROP2 may have a minimum length of one of 10, 20, 30, 40, 50, 100, 150, 200, 250, or 300 amino acids, and may have a maximum length of one of 20, 30, 40, 50, 100, 150, 200, 250, or 300 amino acids.

Isoforms, fragments, variants or homologues may optionally be functional isoforms, fragments, variants or homologues, e.g. having a functional property/activity of the reference TROP2 (e.g. human TROP2), as determined by analysis by a suitable assay for the functional property/activity. For example, an isoform, fragment, variant or homologue of TROP2 may display association with IGF-1, Claudin-1 or Claudin-7.

In some embodiments, the TROP2 is TROP2 from a mammal (e.g. a primate (rhesus, cynomolgous, non-human primate or human) and/or a rodent (e.g. rat or murine) TROP2). Isoforms, fragments, variants or homologues of TROP2 may optionally be characterized as having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of an immature or mature TROP2 isoform from a given species, e.g. human.

In some embodiments, the TROP2 comprises, or consists of, an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to one of SEQ ID NO:1 or 2.

In some embodiments, a fragment of TROP2 comprises, or consists of, an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to SEQ ID NO:2 or 4.

In some embodiments, a fragment of TROP2 comprises, or consists of, an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to one of SEQ ID NOs: 5 to 7. In some embodiments, a fragment of TROP2 comprises, or consists of, an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to SEQ ID NO:67.

### TROP2-Binding Antigen-Binding Moieties

An ‘antigen-binding molecule’ refers to a molecule that binds to a given target antigen. Antigen-binding molecules comprise an antigen-binding moiety through which the antigen-binding molecule binds to its target antigen. The antigen-binding molecules of the present disclosure comprise an antigen-binding moiety that binds to TROP2 (i.e. a TROP2-binding moiety).

Antigen-binding moieties may comprise, or may be derived from, antibodies (i.e. immunoglobulins (Igs)) and antigen-binding fragments of antibodies. As used herein, ‘antibodies’ include monoclonal antibodies, polyclonal antibodies, monospecific and multispecific (e.g., bispecific, trispecific, etc.) antibodies, and antibody-derived antigen-binding molecules such as scFv, scFab, diabodies, triabodies, scFv-Fc, minibodies, single domain antibodies (e.g. VhH, etc.). Antigen-binding fragments of antibodies include e.g. Fv, Fab, F(ab′)₂ and F(ab′) fragments.

Antigen-binding moieties also include target antigen-binding aptamers, e.g. a nucleic acid aptamers (reviewed, for example, in Zhou and Rossi, Nat Rev Drug Discov. (2017) 16 (3): 181-202). In some embodiments, an antigen-binding moiety comprises or consists of an antigen-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)), affilin, armadillo repeat protein (ArmRP), OBody or fibronectin—reviewed e.g. in Reverdatto et al., Curr Top Med Chem. 2015; 15 (12): 1082-1101, which is hereby incorporated by reference in its entirety (see also e.g. Boersma et al., J Biol Chem (2011) 286:41273-85 and Emanuel et al., Mabs (2011) 3:38-48).

Commonly employed techniques for the production of fully human antibodies include (i) phage display, in which human antibody genes are expressed in phage display libraries, and (ii) production of antibodies in transgenic mice engineered to have human antibody genes (described in Park and Smolen, Advances in Protein Chemistry (2001) 56:369-421). Briefly, in the human antibody gene-phage display technique, genes encoding the VH and VL chains are generated by PCR amplification and cloning from ‘naive’ human lymphocytes, and assembled into a library from which they can be expressed either as disulfide-linked Fab fragments or as single-chain Fv (scFv) fragments. The Fab- or scFv-encoding genes are fused to a surface coat protein of filamentous bacteriophage and Fab or scFv capable of binding to the target of interest can then be identified by screening the library with antigen. Molecular evolution or affinity maturation procedures can be employed to enhance the affinity of the Fab/scFv fragment. In the transgenic mouse technique, mice in which the endogenous murine Ig gene loci have been replaced by homologous recombination with their human homologues are immunised with antigen, and monoclonal antibody is prepared by conventional hybridoma technology, to yield a fully human monoclonal antibody.

In some embodiments, an antigen-binding moiety according to the present disclosure comprises, or consists of, the antigen-binding region of an antibody (e.g. an antigen-binding fragment of an antibody). Antigen-binding moieties may be derived from antibodies. Antibody-derived antigen-binding moieties may comprise, or consist of, the antigen-binding region of an antibody (e.g. an antigen-binding fragment of an antibody). In some embodiments, an antigen-binding moiety may be or comprise the Fv (e.g. provided as an scFv) or the Fab region of an antibody that binds to a given target antigen, or the whole antibody.

The antigen-binding moieties of the present disclosure may be designed and prepared using the sequences of monoclonal antibodies (mAbs) capable of binding to a given target antigen (e.g. TROP2). Antigen-binding regions of antibodies, such as variable fragment (Fv), Fab and F(ab′)₂ fragments may also be used/provided. An ‘antigen-binding region’ is any fragment of an antibody that binds to the target for which the given antibody is specific.

In some embodiments, an antigen-binding moiety comprises the antibody heavy chain variable region (VH) and the antibody light chain variable region (VL) of an antibody capable of specific binding to the target antigen. In some embodiments, the antigen-binding moiety is or comprises the Fv (e.g. provided as an scFv) of an antibody. In some embodiments, the antigen-binding moiety is or comprises the Fab region of an antibody. In some embodiments, the antigen-binding moiety is or comprises the whole antibody (i.e. comprising variable and constant regions).

An antigen-binding moiety may be, or may comprise, an antigen-binding polypeptide, or an antigen-binding polypeptide complex. An antigen-binding moiety may comprise more than one polypeptide which together form an antigen-binding moiety. The polypeptides may associate covalently or non-covalently. In some embodiments, the polypeptides form part of a larger polypeptide comprising the polypeptides (e.g. in the case of scFv comprising VH and VL, or in the case of scFab comprising VH-CH1 and VL-CL).

In some embodiments, an antigen-binding moiety according to the present disclosure comprises, or consists of, a polypeptide complex formed by protein:protein interaction between constituent peptides/polypeptides of the antigen-binding moiety. An antigen-binding moiety may refer to a non-covalent or covalent complex of more than one polypeptide (e.g. 2, 3, 4, 6, or 8 polypeptides), e.g. an IgG-like antigen-binding moiety comprising two heavy chain polypeptides and two light chain polypeptides.

Antibodies generally comprise six complementarity-determining regions CDRs; three in the heavy chain variable (VH) region: HC-CDR1, HC-CDR2 and HC-CDR3, and three in the light chain variable (VL) region: LC-CDR1, LC-CDR2, and LC-CDR3. The six CDRs together define the paratope of the antibody, which is the part of the antibody that binds to the target antigen.

The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5th Ed. Public Health Service, National Institutes of Health, Bethesda, MD (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibody clones described herein were defined according to the international IMGT (ImMunoGeneTics) information system (LeFranc et al., Nucleic Acids Res. (2015) 43 (Database issue): D413-22), which uses the IMGT V-DOMAIN numbering rules as described in Lefranc et al., Dev. Comp. Immunol. (2003) 27:55-77. In preferred embodiments, the CDRs and FRs of antigen-binding molecules referred to herein are defined according to the IMGT information system.

In some embodiments, an antigen-binding moiety according to the present disclosure comprises, or consists of, an Fv region that binds to TROP2. In some embodiments, the VH and VL regions of the Fv are provided as single polypeptide joined by a linker sequence, i.e. a single chain Fv (scFv).

The VL and light chain constant (CL) region, and the VH region and heavy chain constant 1 (CH1) region of an antigen-binding region of an antibody together constitute the Fab region. In some embodiments, the antigen-binding moiety comprises a Fab region comprising a VH, a CH1, a VL and a CL (e.g. C_{κ} or C_{λ}). In some embodiments, the Fab region comprises a polypeptide comprising a VH and a CH1 (e.g. a VH-CH1 fusion polypeptide), and a polypeptide comprising a VL and a CL (e.g. a VL-CL fusion polypeptide). In some embodiments, the Fab region comprises a polypeptide comprising a VH and a CL (e.g. a VH-CL fusion polypeptide) and a polypeptide comprising a VL and a CH1 (e.g. a VL-CH1 fusion polypeptide); that is, in some embodiments, the Fab region is a CrossFab region. In some embodiments, the VH, CH1, VL and CL regions of the Fab or CrossFab are provided as single polypeptide joined by linker regions, i.e. as a single chain Fab (scFab) or a single chain CrossFab (scCrossFab).

In some embodiments, an antigen-binding moiety described herein comprises, or consists of, a whole antibody which binds to TROP2. As used herein, ‘whole antibody’ refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125 (202): S41-S52, which is hereby incorporated by reference in its entirety.

Immunoglobulins of type G (i.e. IgG) are ~150 kDa glycoproteins comprising two heavy chains and two light chains. From N- to C-terminus, the heavy chains comprise a VH followed by a heavy chain constant region comprising three constant domains (CH1, CH2, and CH3), and similarly the light chains comprise a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (A).

In some embodiments, the antigen-binding moiety comprises, or consists of, an IgG (e.g. IgG1, IgG2, IgG3, IgG4), IgA (e.g. IgA1, IgA2), IgD, IgE, or IgM which binds to TROP2.

In some embodiments, an antigen-binding moiety of the present disclosure comprises one or more regions (e.g. CH1, hinge, CH2, CH3, etc.) of an immunoglobulin heavy chain constant sequence. In some embodiments, the immunoglobulin heavy chain constant sequence is, or is derived from, the heavy chain constant sequence of an IgG (e.g. IgG1, IgG2, IgG3, IgG4), IgA (e.g. IgA1, IgA2), IgD, IgE or IgM, e.g. a human IgG (e.g. hIgG1, hIgG2, hIgG3, hIgG4), hIgA (e.g. hIgA1, hIgA2), hIgD, hIgE or hIgM. In some embodiments, the immunoglobulin heavy chain constant sequence is, or is derived from, the heavy chain constant sequence of a human IgG1 allotype (e.g. G1 ml, G1m2, G1m3 or G1m17).

In some embodiments, an antigen-binding moiety comprises one or more polypeptides comprising an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:47 or 52. In some embodiments, an antigen-binding moiety comprises one or more polypeptides comprising an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:48. In some embodiments, an antigen-binding moiety comprises one or more polypeptides comprising an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:49 or 64. In some embodiments, an antigen-binding moiety comprises one or more polypeptides comprising an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:50 or 53. In some embodiments, an antigen-binding moiety comprises one or more polypeptides comprising an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:54, 55, 65 or 66. In some embodiments, an antigen-binding moiety comprises one or more polypeptides comprising an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:46, 51, 62 or 63.

In some embodiments, an antigen-binding moiety of the present disclosure comprises one or more regions of an immunoglobulin light chain constant sequence. In some embodiments, the immunoglobulin light chain constant sequence is human immunoglobulin kappa constant (IGKC; C_{K}). In some embodiments, the immunoglobulin light chain constant sequence is a human immunoglobulin lambda constant (IGLC; Cλ), e.g. IGLC1, IGLC2, IGLC3, IGLC6 or IGLC7.

In some embodiments, an antigen-binding moiety comprises one or more polypeptides comprising an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:56, 57, 58, 59, 60, or 61.

In some embodiments described herein, one or more amino acids of an amino acid sequence referred to herein (e.g. an amino acid sequence of an antigen-binding moiety, e.g. an amino acid sequence of a CDR or VH/VL region) are substituted with another amino acid. A substitution comprises substitution of an amino acid residue with a non-identical ‘replacement’ amino acid residue. A replacement amino acid residue of a substitution according to the present disclosure may be a naturally-occurring amino acid residue (i.e. encoded by the genetic code) which is non-identical to the amino acid residue at the relevant position of the equivalent, unsubstituted amino acid sequence, selected from: alanine (Ala), arginine (Arg), asparagine (Asn), aspartic acid (Asp), cysteine (Cys), glutamine (Gln), glutamic acid (Glu), glycine (Gly), histidine (His), isoleucine (lle): leucine (Leu), lysine (Lys), methionine (Met), phenylalanine (Phe), proline (Pro), serine (Ser), threonine (Thr), tryptophan (Trp), tyrosine (Tyr), and valine (Val). In some embodiments, a replacement amino acid may be a non-naturally occurring amino acid residue—i.e. an amino acid residue other than those recited in the preceding sentence. Examples of non-naturally occurring amino acid residues include norleucine, ornithine, norvaline, homoserine, aib, and other amino acid residue analogues such as those described in Ellman, et al., Meth. Enzym. 202 (1991) 301-336.

In some embodiments, a substitution may be biochemically conservative. In some embodiments, where an amino acid to be substituted is provided in one of rows 1 to 5 of the table below, the replacement amino acid of the substitution is another, non-identical amino acid provided in the same row:

| Row | Shared property | Amino acids |
|---|---|---|
| 1 | Hydrophobic | Met, Ala, Val, Leu, Ile, |
| | | Trp, Tyr, Phe, Norleucine |
| 2 | Neutral hydrophilic | Cys, Ser, Thr, Asn, Gln |
| 3 | Acidic or negatively-charged | Asp, Glu |
| 4 | Basic or positively-charged | His, Lys, Arg |
| 5 | Orientation influencing | Gly, Pro |

By way of illustration, in some embodiments wherein substitution is of a Met residue, the replacement amino acid may be selected from Ala, Val, Leu, Ile, Trp, Tyr, Phe and Norleucine.

In some embodiments, a replacement amino acid in a substitution may have the same side chain polarity as the amino acid residue it replaces. In some embodiments, a replacement amino acid in a substitution may have the same side chain charge (at pH 7.4) as the amino acid residue it replaces:

| | | Side-chain charge |
|---|---|---|
| Amino Acid | Side-chain polarity | (pH 7.4) |
| Alanine | nonpolar | neutral |
| Arginine | basic polar | positive |
| Asparagine | polar | neutral |
| Aspartic acid | acidic polar | negative |
| Cysteine | nonpolar | neutral |
| Glutamic acid | acidic polar | negative |
| Glutamine | polar | neutral |
| Glycine | nonpolar | neutral |
| Histidine | basic polar | positive (10%) |
| | | neutral (90%) |
| Isoleucine | nonpolar | neutral |
| Leucine | nonpolar | neutral |
| Lysine | basic polar | positive |
| Methionine | nonpolar | neutral |
| Phenylalanine | nonpolar | neutral |
| Proline | nonpolar | neutral |
| Serine | polar | neutral |
| Threonine | polar | neutral |
| Tryptophan | nonpolar | neutral |
| Tyrosine | polar | neutral |
| Valine | nonpolar | neutral |

That is, in some embodiments, a nonpolar amino acid is substituted with another, non-identical nonpolar amino acid. In some embodiments, a polar amino acid is substituted with another, non-identical polar amino acid. In some embodiments, an acidic polar amino acid is substituted with another, non-identical acidic polar amino acid. In some embodiments, a basic polar amino acid is substituted with another, non-identical basic polar amino acid. In some embodiments, a neutral amino acid is substituted with another, non-identical neutral amino acid. In some embodiments, a positive amino acid is substituted with another, non-identical positive amino acid. In some embodiments, a negative amino acid is substituted with another, non-identical negative amino acid.

In some embodiments, substitution(s) may be functionally conservative. That is, in some embodiments, the substitution may not affect (or may not substantially affect) one or more functional properties (e.g. target binding) of the antigen-binding moiety comprising the substitution as compared to the equivalent unsubstituted molecule.

The antigen-binding molecules of the present disclosure comprise an antigen-binding moiety that binds to TROP2.

In some embodiments, the antigen-binding moiety comprises the CDRs of an antigen-binding moiety which is capable of binding to TROP2. In some embodiments, the antigen-binding moiety comprises the FRs of an antigen-binding moiety which is capable of binding to TROP2. In some embodiments, the antigen-binding moiety comprises the CDRs and the FRs of an antibody that is capable of binding to TROP2. That is, in some embodiments the antigen-binding moiety comprises the VH region and the VL region of an antibody that is capable of binding to TROP2.

In some embodiments, an antigen-binding molecule which is capable of binding to TROP2 according to the present disclosure may be, or may be derived from, a TROP2-binding antibody selected from: hRS7 (Sacituzumab) (described in e.g.WO03074566); MAAP-9001a (Datopotamab) (described in e.g. Okajima et al., Mol Cancer Ther. (2021) 20 (12): 2329-2340; PrE11 (described in e.g. Ikeda et al., Biochemical and Biophysical Research Communications (2015) 458 (4): 877-882); AR47A6.4.2 (described in e.g. Truong et al., Mol Cancer Ther. (2007) 6 (11_Supplement): PR-12); 7E6 (described in e.g. Liu et al., WO2013068946); 2EF (described in e.g. Guerra et al., Cancers (2023) 15 (14): 3721); and Hu2G10 (described in e.g. Albert et al., J. Clin. Oncol. (2022) 40:e14548).

In some embodiments the antigen-binding moiety is capable of binding the same region of TROP2, or an overlapping region of TROP2, to the region of TROP2 which is bound by an antigen-binding molecule comprising the VH and VL sequences of a TROP2-binding antibody described hereinabove. In some embodiments the antigen-binding moiety is capable of binding the same region of TROP2, or an overlapping region of TROP2, to the region of TROP2 which is bound by an antigen-binding molecule comprising the VH and VL sequences of sacituzumab (i.e. an antigen-binding molecule comprising a VH having the amino acid sequence of SEQ ID NO:12, and a VL having the amino acid sequence of SEQ ID NO: 20). In some embodiments the antigen-binding moiety is capable of binding the same region of TROP2, or an overlapping region of TROP2, to the region of TROP2 which is bound by an antigen-binding molecule comprising the VH and VL sequences of datopotamab (i.e. an antigen-binding molecule comprising a VH having the amino acid sequence of SEQ ID NO:30, and a VL having the amino acid sequence of SEQ ID NO:38).

In some embodiments, the antigen-binding moiety is capable of binding to the region of TROP2 shown in SEQ ID NO:67. In some embodiments, the antigen-binding moiety contacts the region of TROP2 shown in SEQ ID NO:67. In some embodiments, the antigen-binding moiety binds to TROP2 via contact with one or more amino acids of the region shown in SEQ ID NO:67. In some embodiments, the epitope of the antigen-binding moiety comprises or consists of the amino acid sequence shown in SEQ ID NO:67. In some embodiments, the antigen-binding moiety binds to a polypeptide comprising or consisting of the amino acid sequence shown in SEQ ID NO:67.

In some embodiments, the antigen-binding moiety is capable of binding to a polypeptide comprising, or consisting of, the amino acid sequence of one of SEQ ID NOs: 1, 2 or 4.

The ability of an antigen-binding moiety to bind to a given peptide/polypeptide can be analysed by methods well known to the skilled person, including analysis by ELISA, immunoblot (e.g. western blot), immunoprecipitation, Surface Plasmon Resonance (SPR; see e.g. Hearty et al., Methods Mol Biol (2012) 907:411-442) or Bio-Layer Interferometry (see e.g. Lad et al., (2015) J Biomol Screen 20 (4): 498-507).

In embodiments where the antigen binding moiety is capable of binding to a peptide/polypeptide comprising a reference amino acid sequence, the peptide/polypeptide may comprise one or more additional amino acids at one or both ends of the reference amino acid sequence. In some embodiments the peptide/polypeptide comprises e.g. 1-5, 1-10, 1-20, 1-30, 1-40, 1-50, 5-10, 5-20, 5-30, 5-40, 5-50, 10-20, 10-30, 10-40, 10-50, 20-30, 20-40 or 20-50 additional amino acids at one or both ends of the reference amino acid sequence. In some embodiments the additional amino acid(s) provided at one or both ends (i.e. the N-terminal and C-terminal ends) of the reference sequence correspond to the positions at the ends of the reference sequence in the context of the amino acid sequence of TROP2.

In some embodiments the antigen-binding moiety is capable of binding to a peptide/polypeptide which is bound by an antibody comprising the VH and VL sequences of sacituzumab (i.e. an antigen-binding molecule comprising a VH having the amino acid sequence of SEQ ID NO:12, and a VL having the amino acid sequence of SEQ ID NO:20). In some embodiments the antigen-binding moiety is capable of binding to a peptide/polypeptide which is bound by an antibody comprising the VH and VL sequences of datopotamab (i.e. an antigen-binding molecule comprising a VH having the amino acid sequence of SEQ ID NO: 30, and a VL having the amino acid sequence of SEQ ID NO:38).

In some embodiments the antigen-binding moiety is capable of binding to a peptide/polypeptide which is bound by an antibody comprising the VH and VL sequences of a TROP2-binding antibody described hereinabove.

In some embodiments, the antigen-binding moiety comprises the heavy chain CDRs and the light chain CDRs of a TROP2-binding antibody described hereinabove. In some embodiments, the antigen-binding moiety comprises the VH and VL of a TROP2-binding antibody described hereinabove. In some embodiments, the antigen-binding moiety comprises the heavy chain polypeptide (i.e. comprising VH, CH1, CH2 and CH3 region sequences) and light chain polypeptide (i.e. comprising VL and CL region sequences) of a TROP2-binding antibody described hereinabove.

In some embodiments, the antigen-binding moiety comprises the heavy chain CDRs and the light chain CDRs of sacituzumab. In some embodiments, the antigen-binding moiety comprises the VH and VL of sacituzumab. In some embodiments, the antigen-binding moiety comprises the heavy chain polypeptide (i.e. comprising VH, CH1, CH2 and CH3 region sequences) and light chain polypeptide (i.e. comprising VL and CL region sequences) of sacituzumab.

In some embodiments, the antigen-binding moiety comprises:
a VH region incorporating the following CDRs:
   HC-CDR1 having the amino acid sequence of SEQ ID NO:13
   HC-CDR2 having the amino acid sequence of SEQ ID NO:14
   HC-CDR3 having the amino acid sequence of SEQ ID NO:15,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid; and
a VL region incorporating the following CDRs:
   LC-CDR1 having the amino acid sequence of SEQ ID NO:21
   LC-CDR2 having the amino acid sequence of SEQ ID NO:22
   LC-CDR3 having the amino acid sequence of SEQ ID NO:23;
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.

In some embodiments, the antigen-binding moiety comprises:
a VH region comprising an amino acid sequence having at least 60% sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity to the amino acid sequence of SEQ ID NO:12; and
a VL region comprising an amino acid sequence having at least 60% sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity to the amino acid sequence of SEQ ID NO:20.

In some embodiments, an antigen-binding moiety comprises, or consists of:
(i) one or more (e.g. two) polypeptides comprising, or consisting of, an amino acid sequence having at least 70% sequence identity, more preferably one of ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:10; and
(ii) one or more (e.g. two) polypeptides comprising, or consisting of, an amino acid sequence having at least 70% sequence identity, more preferably one of ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:11.

In some embodiments, the antigen-binding moiety comprises the heavy chain CDRs and the light chain CDRs of datopotamab. In some embodiments, the antigen-binding moiety comprises the VH and VL of datopotamab. In some embodiments, the antigen-binding moiety comprises the heavy chain polypeptide (i.e. comprising VH, CH1, CH2 and CH3 region sequences) and light chain polypeptide (i.e. comprising VL and CL region sequences) of datopotamab.

In some embodiments, the antigen-binding moiety comprises:
a VH region incorporating the following CDRs:
   HC-CDR1 having the amino acid sequence of SEQ ID NO:31
   HC-CDR2 having the amino acid sequence of SEQ ID NO:32
   HC-CDR3 having the amino acid sequence of SEQ ID NO:33,
   or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid; and
a VL region incorporating the following CDRs:
   LC-CDR1 having the amino acid sequence of SEQ ID NO:39
   LC-CDR2 having the amino acid sequence of SEQ ID NO:40
   LC-CDR3 having the amino acid sequence of SEQ ID NO:41;
   or a variant thereof in which 1 or 2 or 3 amino acids in LC-CDR1, and/or in which 1 or 2 or 3 amino acids in LC-CDR2, and/or in which 1 or 2 or 3 amino acids in LC-CDR3 are substituted with another amino acid.

In some embodiments, the antigen-binding moiety comprises:
a VH region comprising an amino acid sequence having at least 60% sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity to the amino acid sequence of SEQ ID NO:30; and
a VL region comprising an amino acid sequence having at least 60% sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% sequence identity to the amino acid sequence of SEQ ID NO:38.

In some embodiments, an antigen-binding moiety comprises, or consists of:
(i) one or more (e.g. two) polypeptides comprising, or consisting of, an amino acid sequence having at least 70% sequence identity, more preferably one of ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:28; and
(ii) one or more (e.g. two) polypeptides comprising, or consisting of, an amino acid sequence having at least 70% sequence identity, more preferably one of ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:29.

In some embodiments, an antigen-binding molecule of the present disclosure (e.g. an antigen-binding moiety thereof) comprises an Fc region. As used herein, an ‘Fc region’ refers to a polypeptide complex formed by interaction between two polypeptides, each polypeptide comprising the CH2-CH3 region of an immunoglobulin (lg) heavy chain constant sequence.

Herein, a ‘CH2 domain’ refers to an amino acid sequence corresponding to the CH2 domain of an immunoglobulin (lg). The CH2 domain is the region of an Ig formed by positions 231 to 340 of the immunoglobulin constant domain, according to the EU numbering system described in Edelman et al., Proc Natl Acad Sci USA (1969) 63 (1): 78-85. A ‘CH3 domain’ refers to an amino acid sequence corresponding to the CH3 domain of an immunoglobulin (lg). The CH3 domain is the region of an Ig formed by positions 341 to 447 of the immunoglobulin constant domain, according to the EU numbering system described in Edelman et al., Proc Natl Acad Sci USA (1969) 63 (1): 78-85. A ‘CH2-CH3 region’ refers to an amino acid sequence corresponding to the CH2 and CH3 domains of an immunoglobulin (lg). The CH2-CH3 region is the region of an Ig formed by positions 231 to 447 of the immunoglobulin constant domain, according to the EU numbering system described in Edelman et al., Proc Natl Acad Sci USA (1969) 63 (1): 78-85.

In some embodiments, a CH2 domain, CH3 domain and/or a CH2-CH3 region according to the present disclosure corresponds to the CH2 domain/CH3 domain/CH2-CH3 region of an IgG (e.g. IgG1, IgG2, IgG3, IgG4), IgA (e.g. IgA1, IgA2), IgD, IgE or IgM. In some embodiments, the CH2 domain, CH3 domain and/or a CH2-CH3 region corresponds to the CH2 domain/CH3 domain/CH2-CH3 region of a human IgG (e.g. hIgG1, hIgG2, hIgG3, hIgG4), hIgA (e.g. hIgA1, hIgA2), hIgD, hIgE or hIgM. In some embodiments, the CH2 domain, CH3 domain and/or a CH2-CH3 region corresponds to the CH2 domain/CH3 domain/CH2-CH3 region of a human IgG1 allotype (e.g. G1 ml, G1m2, G1m3 or G1m17). In some embodiments, the CH2 domain, CH3 domain and/or a CH2-CH3 region corresponds to the CH2 domain/CH3 domain/CH2-CH3 region of human IgG1 allotype G1m3.

Fc regions provide for interaction with Fc receptors and other molecules of the immune system to bring about functional effects. Fc-mediated effector functions are reviewed e.g. in Jefferis et al., Immunol Rev 1998 163:59-76 (hereby incorporated by reference in its entirety), and are brought about through Fc-mediated recruitment and activation of immune cells (e.g. macrophages, dendritic cells, neutrophils, basophils, eosinophils, platelets, mast cells, NK cells and T cells) through interaction between the Fc region and Fc receptors expressed by the immune cells, recruitment of complement pathway components through binding of the Fc region to complement protein C1q, and consequent activation of the complement cascade. Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

In some embodiments, an antigen-binding molecule according to the present disclosure comprises one or more (e.g. two) polypeptides comprising an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:54 or 55. In some embodiments, the antigen-binding molecule comprises an Fc region comprising one or more (e.g. two) polypeptides comprising an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:54 or 55.

Modifications to antibody Fc regions that influence Fc-mediated functions are known in the art, such as those described e.g. in Wang et al., Protein Cell (2018) 9 (1): 63-73, which is hereby incorporated by reference in its entirety. Exemplary Fc region modifications known to influence antibody effector function are summarised in Table 1 of Wang et al., Protein Cell (2018) 9 (1): 63-73. In some embodiments, the antigen-binding molecule of the present disclosure comprises an Fc region comprising modification to increase or reduce an Fc-mediated function as compared to an antigen-binding molecule comprising the corresponding unmodified Fc region. Where an Fc region/CH2/CH3 is described as comprising modification(s) ‘corresponding to’ reference substitution(s), equivalent substitution(s) in the homologous Fc/CH2/CH3 are contemplated.

In some embodiments, the antigen-binding molecule of the present disclosure comprises an Fc region comprising modification. In some embodiments, the antigen-binding molecule of the present disclosure comprises an Fc region comprising modification in one or more of the CH2 and/or CH3 regions.

In some embodiments, the Fc region comprises modification to reduce/prevent an Fc-mediated function (e.g. ADCC, ADCP, CDC). In some embodiments, the Fc region comprises modification to reduce/prevent ADCC. In some embodiments, the Fc region comprises modification to reduce/prevent CDC. In some embodiments, the Fc region comprises modification to reduce/prevent binding to an Fc receptor. In some embodiments, the Fc region comprises modification to reduce/prevent binding to an Fcγ receptor. In some embodiments, the Fc region comprises modification to reduce/prevent glycosylation of the amino acid residue corresponding to N297.

In some embodiments, the Fc region comprises modification at the amino acid residue corresponding to N297. In some embodiments, the Fc region comprises modification corresponding to N297A or N297Q or N297G as described in Leabman et al., Mabs. (2013) 5:896-903. Substitution of ‘N297’ with ‘A’, ‘G’ or ‘Q’ is known to eliminate glycosylation, and thereby reduce Fc binding to C1q and Fcγ receptors, and thus also reducing CDC and ADCC. In some embodiments, the Fc region comprises modification corresponding to N297A.

In some embodiments, an antigen-binding molecule according to the present disclosure comprises one or more (e.g. two) polypeptides comprising an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:65 or 66. In some embodiments, the antigen-binding molecule comprises an Fc region comprising one or more (e.g. two) polypeptides comprising an amino acid sequence having at least 60% amino acid sequence identity, e.g. one of ≥70%, ≥75%, ≥80%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% amino acid sequence identity to the amino acid sequence of SEQ ID NO:65 or 66.

An antigen-binding molecule according to the present disclosure may comprise a TROP2-binding moiety according to any embodiment described hereinabove, and a linker-payload moiety as described hereinbelow.

### Linker-Payload Moieties

Aspects and embodiments of the present disclosure relate to antigen-binding molecules comprising a linker-payload moiety. As used herein, a linker-payload moiety refers to a moiety comprising one or more payload moieties, and a linker moiety for linking the payload moiety(/ies) to the antigen-binding region of the antigen-binding molecule.

A payload moiety according to the present disclosure comprises or consist of a cytotoxic agent. Payload moieties are described e.g. in Parslow et al., Biomedicines. 2016 September; 4 (3): 14, Goundry and Parker, Org. Process Res. Dev. (2022) 26, 8, 2121-2123, Fu et al., Signal Transduction and Targeted Therapy (2022) 7:93, Wang et al., Acta Pharmaceutica Sinica B (2023) 13 (10): 4025-4059 and Conilh et al., J. Hematol. & Oncol. (2023) 16:3, all of which are hereby incorporated by reference in their entirety.

In particular, the present disclosure relates to antigen-binding molecules comprising at least one linker-payload moiety, wherein the antigen-binding molecule comprises (a) a payload moiety which is a DNA damage response (DDR) inhibitor, and (b) a payload moiety which is a DNA topoisomerase I (TOP1) inhibitor.

Hereinbelow, for conciseness, ‘a payload moiety which is a DDR inhibitor’ may be referred to simply as a ‘DDR inhibitor moiety’, and similarly ‘a payload moiety which is a TOP1 inhibitor’ may be referred to simply as a ‘TOP1 inhibitor moiety’.

The DNA Damage Response (DDR) is a complex network of mechanisms for detecting and repair DNA damage, in order to preserve genomic stability. The DDR is reviewed e.g. in Groelly et al., Nature Reviews Cancer (2023) 23:78-94 and Molinaro et al., Cancers (Basel). (2021) 13 (15): 3819, both of which are hereby incorporated by reference in their entirety.

The detection of DNA damage and initiation of repair pathways is mediated by proteins such as ATM (Ataxia-Telangiectasia Mutated) and ATR (Ataxia-Telangiectasia and Rad3-Related). ATM is a protein kinase activated by double-strand breaks in DNA, and which initiates downstream signaling. ATR is activated by DNA damage and replication stress, and in particular responds to single-strand breaks and stalled DNA replication forks. CHK1 and CHK2 (Checkpoint Kinases 1 and 2) are downstream effectors of ATM and ATR, and phosphorylate various target proteins to stop cell cycle progression, and facilitate DNA repair. PARP (Poly ADP-Ribose Polymerase) is involved in repairing single-strand DNA breaks, helping to recruit repair factors and the formation of repair complexes at the sites of DNA damage. DNA-PK (DNA-Dependent Protein Kinase) helps bring broken DNA ends together for non-homologous end-joining (NHEJ), for repairing double-strand breaks. The DDR is facilitated by cell cycle regulation through WEE1 and PLK1 (Polo-Like Kinase 1). WEE1 is a kinase that phosphorylates and inhibits CDKs (Cyclin-Dependent Kinases), thereby delaying cell cycle progression and allowing more time for DNA damage repair prior to cell division. PLK1 regulates the cell cycle checkpoint and promotes repair processes through phosphorylation of Polθ. RAD51 is an ATPase involved in DNA repair. Ubiquitin-specific proteases (USPs) modulate the DDR by influencing the ubiquitination of proteins involved in the DDR. Protein kinase membrane associated tyrosine/threonine 1 (PKMYT1) regulates cell cycle and participates in DDR-related signaling. Aurora-A may contribute to the G2 DNA damage checkpoint through PLK1 and CDC25B activation, and is important in the mitotic DNA damage response.

Most cancerous cells have a greater dependency on the DDR than non-cancerous cells. DDR inhibitors and their use for the treatment of cancers is described e.g. in Cheng et al., Eur J Med Chem. (2022) 230:114109, Wang et al., Front Immunol. (2022) 13:854730 and Choi and Lee, Int J Mol Sci. (2022) 23 (3): 1701, all of which are hereby incorporated by reference in their entirety.

In some embodiments, a DDR inhibitor moiety according to the present disclosure is, or comprises, a DDR inhibitor selected from:
(a) a PARP inhibitor (e.g. olaparib, rucaparib, niraparib, talazoparib, veliparib, pamiparib, simmiparib, senaparib, SC-10914, 2X-121, AMXI-5001, JPI-547, AZD5305, IDX-1197, TQB-3823, HWH-340, AsiDNA, STP-1002, RBN-2397, fluzoparib, NMS-03305293, AZD9574);
(b) an ATM inhibitor (e.g. CP-466722, KU-55933, KU-60019, KU-59403, AZ31, AZ32, AZD0156, AZD1390, XRD-0394, M4076, M3541, WSD-0628, SYH-2051, IMP-08, SP-1161, INT-6C4/5C4);
(c) an ATR inhibitor (e.g. M6620 (berzosertib), M4344 (VX-803), AZD6738 (ceralasertib), BAY1895344 (elimusertib) RP3500 (camonsertib), ATRN119, ART380, IMP9064, HRS2398, M1774, IMP9064, SC0245, LF0397, NU6027);
(d) a WEE1 inhibitor (e.g. adavosertib, Debio 0123, PD0166285, PD0407824, AZD1775, ZN-c3 (azenosertib), IMP7068, SY4835, SCO191, IMP7068);
(e) a CHK1/2 inhibitor (e.g. CBP-501, prexasertib, MK-8776, GDC-0575, SRA-737, PF-00477736, AZD7762, LY2603618 (rabusertib), LY2880070, XL884, BEBT260, MU380, NU7441, KU-5778);
(f) a DNA-PK inhibitor (e.g. CC-115, LY-3023414, AsiDNA, M3814 (nedisertib, peposertib), VX-984 (M9831), BR-101801, XRD-0394, SL901, XZP-6877, IMP-11, ZL-2201, BR-2006, AZD7648, NU7441);
(g) a PLK1 inhibitor (e.g. BI-6727 (volasertib), PCM-075 (onvansertib), CYC140 (plogosertib));
(h) a Polθ inhibitor (e.g. ART4215, ART6043, novobiocin, RP-6685, RP-3467);
(i) a RAD51 inhibitor (e.g. CYT0851);
(j) an inhibitor of a ubiquitin-specific protease (USP) family enzyme (e.g. an inhibitor of USP11, USP7, USP4, USP37, USP39, USP45, USP24 and/or USP1; e.g. KSQ-4279);
(k) a PKMYT1 inhibitor (e.g. RP6306); and
(l) an Aurora-A inhibitor (e.g. alisertib, WJ05129 (JS112), JAB-2485).

In some embodiments, the DDR inhibitor moiety is, or comprises, ceralasertib.

In some embodiments, the DDR inhibitor moiety is, or comprises, berzosertib:

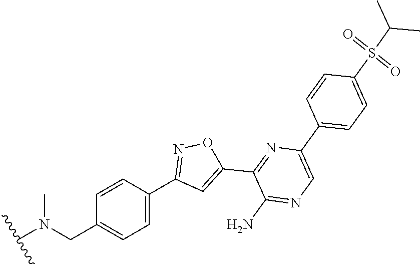 [SMILES: *N(C)Cc1ccc(-c2cc(-c3nc(-c4ccc(S(=O)(=O)C(C)C)cc4)cnc3N)on2)cc1]

During DNA replication and transcription, significant torsional strain is placed on the DNA helix, and this is relieved through the action of DNA topoisomerases I and II (TOP1 and TOP2), which cleave the DNA strand and allow it to untwist, before resealing the breaks (see e.g. Delgado et al., Biochem J. (2018) 475 (2): 373-398). DNA topoisomerase inhibitors block the resealing step, resulting in DNA fragmentation and cell death. DNA topoisomerase I inhibitors and their use for the treatment of cancers is described e.g. in Pommier, Chem Rev. (2009) 109 (7): 2894-2902, Li et al., Am J Cancer Res. (2017) 7 (12): 2350-2394 and Thomas and Pommier, Clin Cancer Res. (2019) 25 (22): 6581-6589, all of which are hereby incorporated by reference in their entirety.

In some embodiments, a TOP1 inhibitor moiety according to the present disclosure is, or comprises, a TOP1 inhibitor selected from: camptothecin or a derivative thereof, exatecan, exatecan mesylate (DX-8951f), N-glycyl-exatecan, SN-38, DXd (1), DXd (2), irinotecan, etirinotecan, FL118, topotecan, gimatecan, belotecan, deruxtecan, belotecan, rubitecan, lurtotecan, diflomotecan, karenitecan, Silat can, namitecan, elomotecan, DRF-1042, delimotecan, NSC606985, chimmitecan, ZBH-1205, Genz-644282, non-CPT1, indotecan (LMP-400), indimitecan (LMP-776), AZ14170132, SHR9265, Ed-04, KL610023, A1.9, ZD06519, P1003, P1021, VIP126, ZBH-01 and LMP-744.

In some embodiments, the TOP1 inhibitor moiety is, or comprises, exatecan:

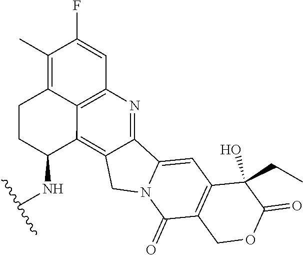 [SMILES: *N[C@H]1CCc2c(C)c(F)cc3nc4c(c1c23)Cn1c-4cc2c(c1=O)COC(=O)[C@]2(O)CC]

A linker moiety according to the present disclosure may be any moiety suitable for linking the payload moiety to the antigen-binding region of the antigen-binding molecule of the present disclosure. Accordingly, they generally comprise a group enabling connection to the payload moiety, a group connecting conjugation to the antigen-binding region of the antigen-binding molecule, and a linker core.

Linker moieties are described e.g. in Su et al., Acta Pharmaceutica Sinica B (2021) 11 (12): 3889-3907, Fu et al., Signal Transduction and Targeted Therapy (2022) 7:93,

A linker moiety according to the present disclosure may be a cleavable linker moiety or a non-cleavable moiety.

Cleavable linkers typically utilise differences between the environment of systemic circulation and that in cancer cells/the tumor microenvironment to release the payload moiety in a targeted manner. Cleavable linkers include chemical cleavage linkers (e.g. acid-cleavable linkers, GSH-cleavable linkers, Fe (II)-cleavable linkers) and enzyme cleavage linkers (e.g. cathepsin-cleavable linkers, glycosidase-cleavable linkers, phosphatase-cleavable linkers, sulfatase-cleavable linkers).

In some embodiments, a linker moiety according to the present disclosure is a chemical cleavage linker. In some embodiments, a linker moiety according to the present disclosure is an enzyme cleavage linker. In some embodiments, a linker moiety is an acid-cleavable linker, e.g. comprising a hydrazone group (e.g. a 6-maleimidocaproylhydrazone linker or a (4-(4-acetylphenoxy) butanoic acid) hydrazaone linker), a carbonate group or a silyl ether group. In some embodiments, a linker moiety is a GSH-cleavable linker, e.g. comprising a disulfide group. In some embodiments, a linker moiety is a Fe (II)-cleavable linker, e.g. comprising a 1,2,4-trioxolane group. In some embodiments, a linker moiety is a cathepsin-cleavable linker, e.g. comprising a dipeptide (e.g. a valine-citrulline linker, a phenylalanine-lysine linker or a valine-alanine linker), a triglycyl peptide (CX) or a cBu-Cit group. In some embodiments, a linker moiety is a glucuronidase-cleavable linker, e.g. comprising a β-glucuronide group. In some embodiments, a linker moiety is a glycosidase-cleavable linker, e.g. comprising a β-galactoside group. In some embodiments, a linker moiety is a phosphatase-cleavable linker, e.g. comprising a pyrophosphate group. In some embodiments, a linker moiety is a sulfatase-cleavable linker, e.g. comprising an arylsulfate group. In some embodiments, a linker moiety is a photo-responsive linker, e.g. comprising a heptamethine cyanine fluorophore group, an O-nitrobenzyl group or a PC4AP group. In some embodiments, a linker moiety is a biorthogonal cleavable linker, e.g. comprising a dsProc group.

Non-cleavable linkers remain inert in common chemical and enzymatic environments in the body, with the payload moiety being released following processing of the ADC by cellular lysosomal proteases. Non-cleavable linkers include linkers comprising thioether or maleimidocaproyl groups.

In some embodiments, a linker moiety is a thioether linker. In some embodiments, a linker moiety is a maleimidocaproyl linker, e.g. comprising a 2-(maleimidomethyl)-1,3-dioxane (MD) group or a Mal-PAB group. In some embodiments, a linker moiety comprises a polyethylene glycol (PEG) group and an alkyne, triazole or piperazine group.

In some embodiments, a linker-payload moiety according to the present disclosure has an amino (—NH2) group for linkage to the antigen-binding moiety, for example by enzymatic conjugation. In some of these embodiments, enzymatic conjugation with microbial transglutaminase may be used to conjugate the linker-payload moiety to the antigen-binding moiety.

In some embodiments, a linker moiety further comprises a spacer moiety. Spacer moieties are sometimes required sue to the bulky nature of payload moieties. Commonly employed spacer moieties include para-aminobenzyl carbamate (PABC), hemiaminal groups, PEG groups, polar acyl sulfamide groups, polar carbamoyl sulfamide groups and HydraSpace (described e.g. in Verkade et al., Antibodies (Basel) (2018) 7 (1): 12 and WO 2016/053107 A1, both of which are hereby incorporated by reference in their entirety). PABC is commonly employed as a spacer moiety in cathepsin-cleavable dipeptide linkers, β-glucuronidase-cleavable linkers, β-galactosidase-cleavable linkers and phosphatase cleavable linkers.

In some embodiments, an antigen-binding molecule according to the present disclosure comprises a linker-payload moiety, which in turn comprises both a DDR inhibitor moiety and a TOP1 inhibitor moiety. That is, in some embodiments, the antigen-binding molecule comprises a linker-payload moiety comprising: (a) a DDR inhibitor moiety, and (b) a TOP1 inhibitor moiety. In some embodiments, the DDR inhibitor moiety and TOP1 inhibitor moieties are provided in the same linker-payload moiety. In some embodiments, the DDR inhibitor moiety and the TOP1 inhibitor moiety are connected to the antigen-binding moiety of the antigen-binding molecule via the same linker moiety.

Approaches for the attachment of multiple payload moieties to a single linker moiety are described e.g. in Yamazaki, et al., Nat Commun. (2021) 12 (1): 3528, Kumar et al., Bioorg Med Chem Lett (2018) 28 (23-24): 3617-3621, Levengood, et al., Angew Chem Int Ed Engl (2017) 56 (3): 733-737 and Wang et al., Acta Pharmaceutica Sinica B (2023) 13 (10): 4025-4059, all of which are hereby incorporated by reference in their entirety.

In some embodiments, the DDR inhibitor and TOP1 inhibitor payload moieties are provided in the same linker-payload moiety. In some embodiments, the DDR inhibitor and TOP1 inhibitor payload moieties are connected to the antigen-binding moiety of the antigen-binding molecule through the same linker moiety.

In some embodiments, the DDR inhibitor and TOP1 inhibitor payload moieties are connected to the antigen-binding moiety of the antigen-binding molecule through a branched linker moiety. The linker may comprise of multiple branches to allow for DAR flexibility.

In some embodiments, the DDR inhibitor and TOP1 inhibitor payload moieties are connected to the antigen-binding moiety of the antigen-binding molecule through a linker moiety that has increased hydrophilicity.

In some embodiments, the DDR inhibitor and TOP1 inhibitor payload moieties are connected to the antigen-binding moiety of the antigen-binding molecule through a branched hydrophilic linker moiety.

In some embodiments, the DDR inhibitor moiety and the TOP1 inhibitor moiety are connected to the linker moiety of the linker-payload moiety via orthogonal functional groups. In some embodiments, the linker-payload moiety comprises a trifunctional linker moiety providing for linkage of an antigen-binding moiety to two different payload moieties. Such approaches to the production of a linker-payload moiety comprising two different payloads are described e.g. in Yamazaki, et al., Nat Commun. (2021) 12 (1): 3528 and Kumar et al., Bioorg Med Chem Lett (2018) 28 (23-24): 3617-3621.

Kumar et al., Bioorg Med Chem Lett (2018) 28 (23-24): 3617-3621 describes a branched linker moiety comprising:
(a) a group for connection to an antigen-binding moiety (which is a self-stabilizing N-aryl maleimide); and
(b) two orthogonal functional groups for linking payload moieties, which are:
   (i) an alkyne group, for incorporation of a payload moiety via copper-mediated azide-alkyne cycloaddition (CuAAC), and
   (ii) a ketone group, for incorporation of a payload moiety via for aminooxy reaction resulting in oxime linkage.

The branched linker moiety described in Kumar et al., Bioorg Med Chem Lett (2018) 28 (23-24): 3617-3621 has the following structure:

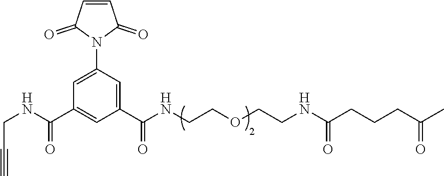

Accordingly, in some embodiments a linker-payload moiety according to the present disclosure comprises a linker moiety comprising: (i) a moiety derived from a group for connection to the antigen-binding moiety (e.g. a self-stabilizing N-aryl maleimide group), (ii) a moiety derived from an alkyne group suitable for incorporating a first payload moiety via CuAAC (i.e. a divalent triazole), and (iii) a moiety derived from a ketone group suitable for incorporating a second payload moiety via aminooxy reaction resulting in oxime linkage (i.e. an oxime). In accordance with such embodiments, in some cases the first payload moiety is a DDR inhibitor moiety as described herein, and the second payload moiety is a TOP1 inhibitor moiety as described herein. In some embodiments, the first payload moiety is a TOP1 inhibitor moiety as described herein, and the second payload moiety is a DDR inhibitor moiety as described herein.

In some embodiments, an antigen-binding molecule according to the present disclosure comprises a linker-payload moiety comprising: (i) a first payload moiety conjugated to the linker moiety via a CuAAC reaction between an azide group and an alkyne group, and (ii) a second payload moiety conjugated to the linker moiety oxime linkage between an alkoxyamine or hydrazide group, and a ketone group. In accordance with such embodiments, in some cases the first payload moiety is a DDR inhibitor moiety as described herein, and the second payload moiety is a TOP1 inhibitor moiety as described herein. In some embodiments, the first payload moiety is a TOP1 inhibitor moiety as described herein, and the second payload moiety is a DDR inhibitor moiety as described herein.

Yamazaki, et al., Nat Commun. (2021) 12 (1): 3528 describes a branched linker moiety comprising:
(a) a group for connection to an antibody (which is lysine-based); and
(b) two orthogonal functional groups for linking payload moieties, which are:
   (i) one or two azide groups for incorporation of a payload moiety via strain-promoted azide-dibenzocyclooctyne (DBCO) cycloaddition; and
   (ii) a methyltetrazine group for incorporation of a payload moiety via trans-cyclooctene (TCO) cycloaddition.

The branched linker moiety described in Yamazaki, et al., Nat Commun. (2021) 12 (1): 3528 has the following structure:

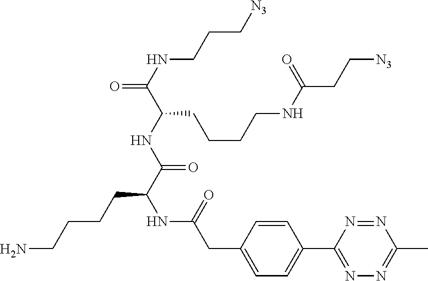 [SMILES: Cc1nnc(-c2ccc(CC(=O)N[C@@H](CCCCN)C(=O)N[C@@H](CCCCNC(=O)CCN=[N+]=[N-])C(=O)NCCCN=[N+]=[N-])cc2)nn1]

Accordingly, in some embodiments a linker-payload moiety according to the present disclosure comprises a linker moiety comprising: (i) a moiety derived from a group for connection to the antigen-binding moiety (e.g. a lysine-based group), (ii) one or two moieties derived from azide groups suitable for incorporating a first payload moiety via DBCO cycloaddition, and (iii) a moiety derived from a methyltetrazine group for incorporation of a second payload moiety via TCO cycloaddition. In accordance with such embodiments, in some cases the first payload moiety is a DDR inhibitor moiety as described herein, and the second payload moiety is a TOP1 inhibitor moiety as described herein. In some embodiments, the first payload moiety is a TOP1 inhibitor moiety as described herein, and the second payload moiety is a DDR inhibitor moiety as described herein.

In some embodiments, an antigen-binding molecule according to the present disclosure comprises a linker-payload moiety comprising: (i) a first payload moiety conjugated to the linker moiety via a DBCO cycloaddition reaction between a DBCO group and an azide group; and (ii) a second payload moiety conjugated to the linker moiety via a TCO cycloaddition reaction between a TCO group and a methyltetrazine group. In accordance with such embodiments, in some cases the first payload moiety is a DDR inhibitor moiety as described herein, and the second payload moiety is a TOP1 inhibitor moiety as described herein. In some embodiments, the first payload moiety is a TOP1 inhibitor moiety as described herein, and the second payload moiety is a DDR inhibitor moiety as described herein.

In some embodiments, the DDR inhibitor moiety and the TOP1 inhibitor moiety are connected to a linker moiety of the linker-payload moiety via cysteine groups. Such an approach to the production of a linker-payload moiety comprising two different payloads is described e.g. in Levengood, et al., Angew Chem Int Ed Engl (2017) 56 (3): 733-737.

Levengood, et al., Angew Chem Int Ed Engl (2017) 56 (3): 733-737 describes a linker-payload moiety comprising two different payload moieties, constructed by sequential deprotection of orthogonally-protected cysteines. Each payload is connected to a maleimide group (for example with a cleavable linker), and the maleimide undergoes a Michael reaction with the deprotected cysteines. The branched linker moiety described in Levengood, et al., Angew Chem Int Ed Engl (2017) 56 (3): 733-737 has the following structure:

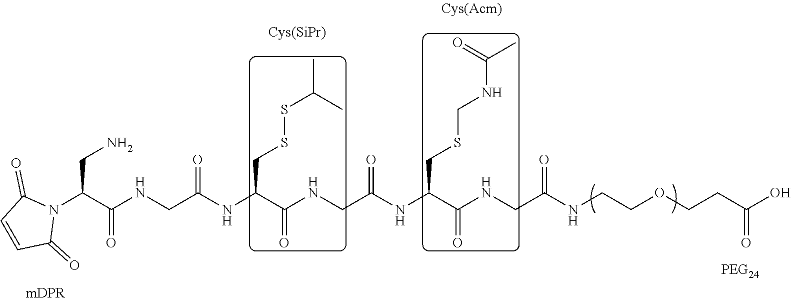

Accordingly, in some embodiments a linker-payload moiety according to the present disclosure comprises: (i) a first payload moiety conjugated to the linker-payload moiety via reduction of a cysteine residue bearing a protecting disulfide group (e.g. a S-(tert-butyl) disulfide group or S-(isopropyl) disulfide group), and subsequent incorporation of the first payload moiety via thiol-maleimide reaction; and (ii) a second payload moiety conjugated to the linker-payload moiety via reduction of a cysteine residue bearing a protecting acetamidomethyl group, and subsequent incorporation of the first payload moiety via thiol-maleimide reaction. In accordance with such embodiments, in some cases the first payload moiety is a DDR inhibitor moiety as described herein, and the second payload moiety is a TOP1 inhibitor moiety as described herein. In some embodiments, the first payload moiety is a TOP1 inhibitor moiety as described herein, and the second payload moiety is a DDR inhibitor moiety as described herein.

In some embodiments, the DDR inhibitor moiety and the TOP1 inhibitor moiety of the antigen-binding molecule of the present disclosure are connected to the antigen-binding moiety of the antigen-binding molecule via different linker moieties.

In some embodiments, an antigen-binding molecule according to the present disclosure comprises: (a) a linker-payload moiety comprising a DDR inhibitor moiety, and (b) a linker-payload moiety comprising a TOP1 inhibitor moiety. That is, in some embodiments, the antigen-binding molecule comprises at least two linker-payload moieties, wherein one of the linker-payload moieties comprises a DDR inhibitor moiety, and wherein another of the linker-payload moieties comprises a TOP1 inhibitor moiety.

Approaches for the attachment of multiple payload moieties to an antigen-binding moiety via multiple linker moieties are described e.g. in Świderska et al., Intl J Mol Sci (2018) 19:2098, Nilchan et al., Antib. Ther. (2019) 2:71-78 and Wang et al., Acta Pharmaceutica Sinica B (2023) 13 (10): 4025-4059, all of which are hereby incorporated by reference in their entirety.

Świderska et al., Intl J Mol Sci (2018) 19:2098 describes an approach in which (i) a first linker-payload moiety (specifically maleimide-Val-Cit-PAB-a-amanitin) is conjugated to a cysteine residue of a polypeptide via thiol-maleimide reaction, and in which (ii) a second linker-payload moiety (specifically an azide linked to MMAE) is conjugated to the same polypeptide via CuAAC-mediated conjugation to the alkyne group of an engineered N-propargyl-L-lysine (PrK) residue.

Accordingly, in some embodiments an antigen-binding molecule according to the present disclosure comprises: (i) a first linker-payload moiety conjugated to the antigen-binding moiety via thiol-maleimide reaction between a cysteine residue of the antigen-binding moiety and a maleimide group of the linker-payload moiety, and (ii) a second linker-payload moiety conjugated via CuAAC reaction between an azide group of the linker-payload moiety to the alkyne group of an N-propargyl-L-lysine residue of the antigen-binding moiety. In accordance with such embodiments, in some cases the first linker-payload moiety comprises a DDR inhibitor moiety as described herein, and the second linker-payload moiety comprises a TOP1 inhibitor moiety as described herein. In some embodiments, the first linker-payload moiety comprises a TOP1 inhibitor moiety as described herein, and the second linker-payload moiety comprises a DDR inhibitor moiety as described herein.

Nilchan et al., Antib. Ther. (2019) 2:71-78 describes a dual conjugation approach in which (i) a first linker-payload moiety is conjugated to an antigen-binding moiety via selenoether conjugation between a selenocysteine residue of the antigen-binding moiety and an iodoacetamide group of the linker-payload moiety, and in which (ii) a second linker-payload moiety is conjugated to the same antigen-binding moiety via reaction between a cysteine residue of the antigen-binding moiety and a methylsulfone phenyloxadiazole (MSODA) group of the linker-payload moiety.

Accordingly, in some embodiments an antigen-binding molecule according to the present disclosure comprises: (i) a first linker-payload moiety conjugated to the antigen-binding moiety via selenoether conjugation between a selenocysteine residue of the antigen-binding moiety and an iodoacetamide group of the linker-payload moiety, and (ii) a second linker-payload moiety conjugated via reaction between a cysteine residue of the antigen-binding moiety and a MSODA group of the linker-payload moiety. In accordance with such embodiments, in some cases the first linker-payload moiety comprises a DDR inhibitor moiety as described herein, and the second linker-payload moiety comprises a TOP1 inhibitor moiety as described herein. In some embodiments, the first linker-payload moiety comprises a TOP1 inhibitor moiety as described herein, and the second linker-payload moiety comprises a DDR inhibitor moiety as described herein.

A further aspect of the present disclosure provides a DDR inhibitor moiety linked to click group, wherein the click group is suitable for conjugation to a corresponding click group in a linker moiety, and wherein the linker moiety is conjugated, or is suitable for conjugation, to an antigen-binding moiety.

A further aspect of the present disclosure provides a TOP1 inhibitor moiety linked to click group, wherein the click group is suitable for conjugation to a corresponding click group in a linker moiety, and wherein the linker moiety is conjugated, or is suitable for conjugation, to an antigen-binding moiety.

In accordance with such aspects, in some embodiments, the click group is selected from:
(i) an azide group;
(ii) an alkyne;
(iii) a tetrazine or tetrazine derivative group;
(iv) a cyclooctyne, cyclooctyne derivative, or cyclooctyne analogue group; and
(v) a strained alkene.

Cyclooctyne, cyclooctyne derivatives and cyclooctyne analogues for use in the present disclosure include:

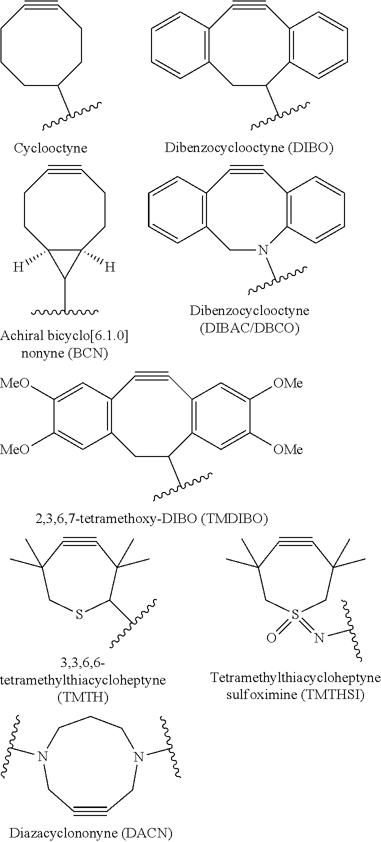

Tetrazine and tetrazine derivatives for use in the present disclosure include:

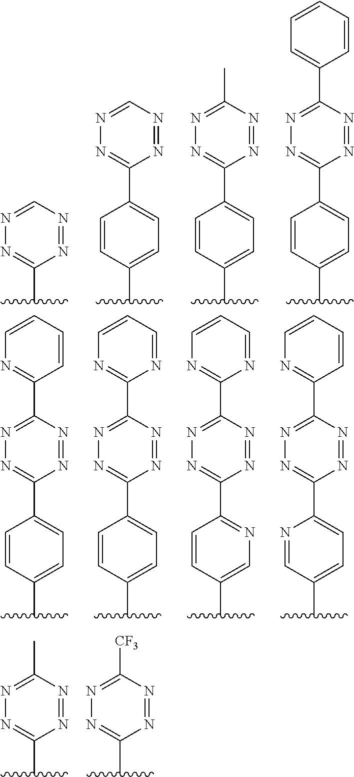

Strained alkenes for use in the present disclosure may have the structure:

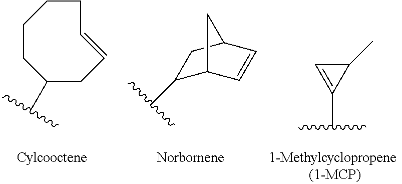

In some embodiments, the click group may be a dibenzoazacyclooctyne (DIBAC) group or a 1-ethylhept-6-enoxy group:

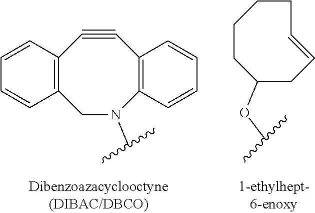

The link between the payload moiety (e.g. the DDR inhibitor moiety or the TOP1 inhibitor moiety) and the click group according to the present disclosure may be a cleavable linker moiety or a non-cleavable moiety, e.g. as described hereinabove.

In some embodiments, the link between the payload moiety (e.g. the DDR inhibitor moiety or the TOP1 inhibitor moiety) and the click group comprises: PABC, a cathepsin-cleavable dipeptide, and a PEG2 to PEG4 (e.g. a PEG3) group. In some of these embodiments, the link between the payload moiety and the click group comprises, or is:

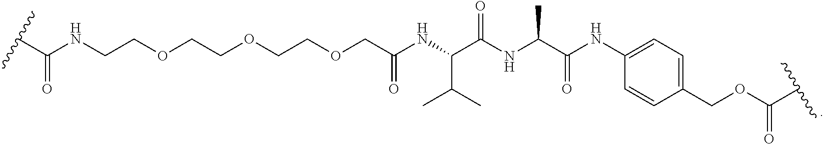 [SMILES: *C(=O)NCCOCCOCCOCC(=O)N[C@H](C(=O)N[C@@H](C)C(=O)Nc1ccc(COC(*)=O)cc1)C(C)C]

In some embodiments, the TOP1 inhibitor moiety linked to click group has the structure:

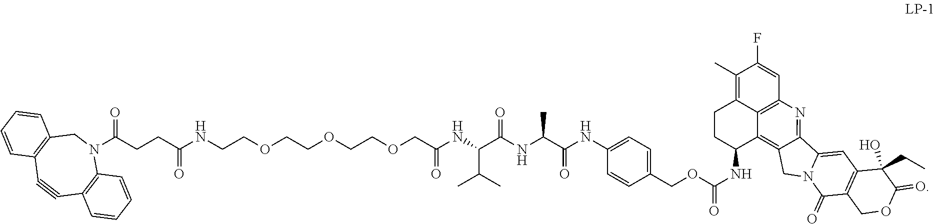 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)COCCOCCOCCNC(=O)CCC(=O)N2Cc4ccccc4C#Cc4ccccc42)C(C)C)cc1)CC3]

In some embodiments, the DDR inhibitor moiety linked to click group has the structure:

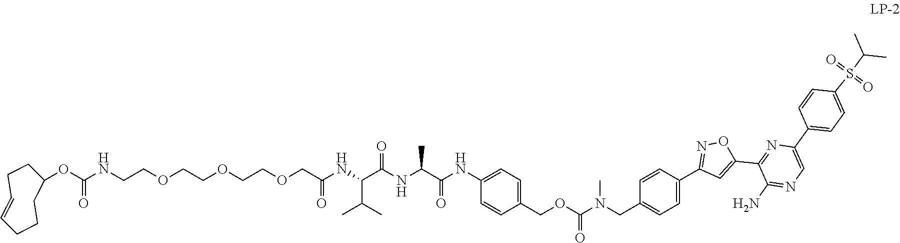 [SMILES: CC(C)[C@H](NC(=O)COCCOCCOCCNC(=O)OC1CC/C=C/CCC1)C(=O)N[C@@H](C)C(=O)Nc1ccc(COC(=O)N(C)Cc2ccc(-c3cc(-c4nc(-c5ccc(S(=O)(=O)C(C)C)cc5)cnc4N)on3)cc2)cc1]

In some embodiments, the DDR inhibitor moiety linked to click group has the structure:

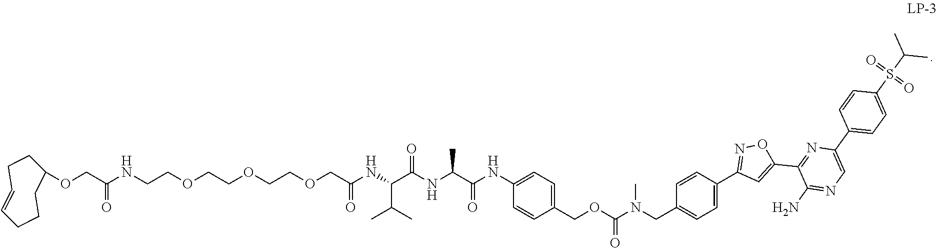 [SMILES: CC(C)[C@H](NC(=O)COCCOCCOCCNC(=O)COC1CC/C=C/CCC1)C(=O)N[C@@H](C)C(=O)Nc1ccc(COC(=O)N(C)Cc2ccc(-c3cc(-c4nc(-c5ccc(S(=O)(=O)C(C)C)cc5)cnc4N)on3)cc2)cc1]

### Functional Properties of the Antigen-Binding Molecules

The antigen-binding molecules described herein may be characterised by reference to certain functional properties. In some embodiments, an antigen-binding molecule described herein may possess one or more of the following properties:
binds to cells expressing TROP2;
inhibits proliferation of TROP2-expressing cells;
increases killing of cells expressing TROP2;
inhibits tumor growth and/or reduces tumor size/volume (e.g. of a TROP2-expressing cancer);
increases survival of subjects having a cancer (e.g. a TROP2-expressing cancer).

It will be appreciated that a given antigen-binding molecule may display more than one of the properties recited in the preceding paragraph. A given antigen-binding molecule may be evaluated for the properties recited in the preceding paragraph using suitable assays. For example, the assays may be e.g. in vitro assays, optionally cell-based assays or cell-free assays. In some embodiments, the assays may be e.g. in vivo assays, i.e. performed in non-human animals. In some embodiments, the assays may be e.g. ex vivo assays, i.e. performed using cells/tissue/an organ obtained from a subject.

Where assays are cell-based assays, they may comprise treating cells with an antigen-binding molecule in order to determine whether the antigen-binding molecule displays one or more of the recited properties. Assays may employ species labelled with detectable entities in order to facilitate their detection. Assays may comprise evaluating the recited properties following treatment of cells separately with a range of quantities/concentrations of a given antigen-binding molecule (e.g. a dilution series).

Analysis of the results of such assays may comprise determining the concentration at which 50% of the maximal level of the relevant activity is attained. The concentration of a given agent at which 50% of the maximal level of the relevant activity is attained may be referred to as the ‘half-maximal effective concentration’ of the agent in relation to the relevant activity, which may also be referred to as the ‘EC₅₀’. Depending on the property, the EC₅₀ may also be referred to as the ‘half-maximal inhibitory concentration’ or ‘IC₅₀’, this being the concentration of the agent at which 50% of the maximal level of inhibition of a given property is observed.

In some embodiments, the antigen-binding molecule of the present disclosure binds to TROP2 in a region which is accessible to an antigen-binding molecule (i.e., an extracellular antigen-binding molecule) when TROP2 is expressed at the cell surface (i.e. in or at the cell membrane). In some embodiments, the antigen-binding molecule binds to TROP2 expressed at the cell surface of a cell expressing TROP2. In some embodiments, the antigen-binding molecule binds to TROP2-expressing cells.

The ability of an antigen-binding molecule to bind to a given cell type can be analysed by contacting cells with the antigen-binding molecule, and detecting antigen-binding molecule bound to the cells, e.g. after a washing step to remove unbound antigen-binding molecule. The ability of an antigen-binding molecule to bind to TROP2-expressing cells can be analysed by methods such as flow cytometry and immunofluorescence microscopy.

In some embodiments, the antigen-binding molecule inhibits proliferation of TROP2-expressing cells (e.g. TROP2-expressing cancer cells). The ability of an antigen-binding molecule to inhibit proliferation of a given cell type can be analysed by contacting cells with the antigen-binding molecule, and subsequently evaluating proliferation of the cells (i.e. after a period of time sufficient for an effect on cell proliferation to be observed). Cell proliferation can be evaluated e.g. by detecting changes in number of cells over time, or by in vitro analysis of incorporation of 3H-thymidine or by CFSE dilution assay, e.g. as described in Fulcher and Wong, Immunol Cell Biol (1999) 77 (6): 559-564, hereby incorporated by reference in entirety.

In some embodiments, the antigen-binding molecule of the present invention is capable of inhibiting proliferation of TROP2-expressing cells to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of proliferation of TROP2-expressing cells observed in the absence of the antigen-binding molecule (or in the presence of an appropriate control antigen-binding molecule known not to influence proliferation of TROP2-expressing cells), in a given assay.

In some embodiments, the antigen-binding molecule described herein inhibits proliferation of cells expressing human TROP2 with an IC₅₀ of 100 nM or less, preferably one of ≤50 nM, ≤40 nM, ≤30 nM, ≤20 nM, ≤10 nM, ≤5 nM, ≤4 nM, ≤3 nM, ≤2 nM, ≤1 nM, ≤900 μM, ≤800 μM, ≤700 μM, ≤600 μM or ≤500 pM.

In some embodiments, the antigen-binding molecule according to the present disclosure potentiates (i.e. upregulates, enhances) cell killing of cells comprising/expressing TROP2.

In some embodiments, an antigen-binding molecule according to the present disclosure may inhibit growth or reduce metastasis of a cancer comprising cells comprising/expressing TROP2. In some embodiments, an antigen-binding molecule may potentiate (i.e. upregulate, enhance) cell killing of cells comprising/expressing TROP2. In some embodiments, an antigen-binding molecule may inhibit growth of cells of a cancer, or may inhibit growth of a tumor, comprising cells comprising/expressing TROP2. In some embodiments, an antigen-binding molecule may inhibit metastasis of a cancer/tumor comprising cells comprising/expressing TROP2.

Cell killing can be investigated, for example, using any of the methods reviewed in Zaritskaya et al., Expert Rev Vaccines (2011), 9 (6): 601-616, hereby incorporated by reference in its entirety. Examples of in vitro assays of cytotoxicity/cell killing assays include release assays such as the ⁵¹Cr release assay, the lactate dehydrogenase (LDH) release assay, the 3-(4,5-dimethylthiazol-2-yl)-2,5-diphenyl tetrazolium bromide (MTT) release assay, ATP release assay using Cell Titre Glo, and the calcein-acetoxymethyl (calcein-AM) release assay. These assays measure cell killing based on the detection of factors released from lysed cells.

In some embodiments an antigen-binding molecule according to the present disclosure is capable of reducing the number/proportion of cells expressing TROP2. In some embodiments, an antigen-binding molecule according to the present disclosure is capable of depleting/enhancing depletion of such cells.

In some embodiments, an antigen-binding molecule of the present disclosure displays anticancer activity. In some embodiments, the antigen-binding molecule increases killing of cancer cells. In some embodiments, the antigen-binding molecule causes a reduction in the number of cancer cells in vivo, e.g. as compared to an appropriate control condition. The cancer may be a cancer as described herein, e.g. a cancer expressing/overexpressing TROP2.

In some embodiments, an antigen-binding molecule according to the present disclosure reduces/inhibits growth of a cancer and/or of a tumor of a cancer. In some embodiments, an antigen-binding molecule reduces tissue invasion by cells of a cancer. In some embodiments, an antigen-binding molecule reduces metastasis of a cancer. In some embodiments, an antigen-binding molecule displays anticancer activity. In some embodiments, an antigen-binding molecule reduces the growth/proliferation of cancer cells. In some embodiments, an antigen-binding molecule reduces the survival of cancer cells. In some embodiments, an antigen-binding molecule increases the killing of cancer cells. In some embodiments, an antigen-binding molecule of the present disclosure causes a reduction in the number of cancer cells e.g. in vivo. The cancer may be a cancer comprising cells expressing TROP2.

An antigen-binding molecule of the present disclosure may be analysed for the properties described in the preceding paragraph in appropriate assays. Such assays include e.g. in vivo models.

In some embodiments, administration of an antigen-binding molecule according to the present disclosure may cause one or more of: inhibition of the development/progression of the cancer, a delay to/prevention of onset of the cancer, a reduction in/delay to/prevention of tumor growth, a reduction in/delay to/prevention of tissue invasion, a reduction in/delay to/prevention of metastasis, a reduction in the severity of one or more symptoms of the cancer, a reduction in the number of cancer cells, a reduction in the cancer burden, a reduction in tumor size/volume, and/or an increase in survival of subjects having the cancer (e.g. progression free survival or overall survival), e.g. as determined in an appropriate model.

It will be appreciated that the properties recited in the preceding paragraph are evaluated after a period of time sufficient for an effect associated with treatment using the antigen-binding molecule to be observed. Tumor growth may be monitored by investigating tumor volume over time. Tumor growth may be evaluated by measuring tumor volume (e.g. in mm³) over time.

In some embodiments, an antigen-binding molecule of the present disclosure is capable of reducing tumor size/volume (e.g. the mean tumor size/volume for the treatment group in an in vivo model, e.g. of a TROP2-expressing cancer) to less than 1 times, e.g. ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the tumor size/volume observed at the same time point in the absence of treatment with the antigen-binding molecule (or following treatment with an appropriate control antigen-binding molecule known not to influence tumor growth), in a given assay. In some embodiments, evaluation of tumor size/volume for the purposes of such comparison is performed after more than 5 days, e.g. one of ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, ≥85 days, ≥90 days, ≥95 days or ≥100 days following administration of the first dose of the antigen-binding molecule, in the relevant model.

In some embodiments, an antigen-binding molecule of the present disclosure achieves a level of tumor growth inhibition (e.g. expressed as % tumor growth inhibition, e.g. calculated relative to tumor growth observed on treatment with an appropriate control antigen-binding molecule) which is greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level of tumor growth inhibition observed at the same time point in the absence of treatment with the antigen-binding molecule (or following treatment with an appropriate control antigen-binding molecule known not to influence tumor growth), in a given assay. In some embodiments, evaluation of tumor growth inhibition for the purposes of such comparison is performed after more than 5 days, e.g. one of ≥10 days, ≥15 days, ≥20 days, ≥25 days, ≥30 days, ≥35 days, ≥40 days, ≥35 days, ≥50 days, ≥55 days, ≥60 days, ≥65 days, ≥70 days, ≥75 days, ≥80 days, 285 days, ≥90 days, 295 days or ≥100 days following administration of the first dose of the antigen-binding molecule, in the relevant model.

In some embodiments, an antigen-binding molecule of the present disclosure is capable of increasing median survival of subjects having a cancer (e.g. in an in vivo model, e.g. of a TROP2-expressing cancer) to greater than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the median survival observed in the absence of treatment with the antigen-binding molecule (or following treatment with an appropriate control antigen-binding molecule known not to influence survival of subjects having the cancer), in a given assay. Median survival may be expressed in days from the start of the experiment, for subjects in the relevant treatment groups.

### Additional Sequences

The antigen-binding molecules of the present disclosure and their constituent polypeptides may additionally comprise further amino acids or sequences of amino acids.

The polypeptides of the present disclosure may comprise one or more linker sequences between sequences of amino acids. Linker sequences are known to the skilled person, and are described, for example in Chen et al., Adv Drug Deliv Rev (2013) 65 (10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65 (10): 1357-1369. Flexible linker sequences often comprise high proportions of glycine and/or serine residues.

In some embodiments, the linker sequence comprises at least one glycine residue and/or at least one serine residue. In some embodiments, the linker sequence comprises or consists of glycine and serine residues. In some embodiments, the linker sequence has the structure: (GxS) n or (GxS) nGm; wherein G=glycine, S=serine, x=3 or 4, n=2, 3, 4, 5 or 6, and m=0, 1, 2 or 3. In some embodiments, the linker sequence comprises one or more (e.g. 1, 2, 3, 4, 5 or 6) copies (e.g. in tandem) of the sequence motif G4S. In some embodiments, the linker sequence comprises or consists of (G4S) 4 or (G4S) 6. In some embodiments, the linker sequence has a length of 1-2, 1-3, 1-4, 1-5, 1-10, 1-15, 1-20, 1-25, or 1-30 amino acids.

The antigen-binding molecules of the present disclosure and their constituent polypeptides may comprise amino acid sequence(s) to facilitate expression, folding, trafficking, processing, purification or detection of the antigen-binding molecule/polypeptide. For example, antigen-binding molecules and polypeptides of the present disclosure may additionally comprise a sequence of amino acids forming a detectable moiety, e.g. as described hereinbelow.

The antigen-binding molecules of the present disclosure and their constituent polypeptides may additionally comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides. Signal peptides are known for many proteins, and are recorded in databases such as GenBank, UniProt and Ensembl, and/or can be identified/predicted e.g. using amino acid sequence analysis tools such as SignalP (Petersen et al., 2011 Nature Methods 8:785-786) or Signal-BLAST (Frank and Sippl, 2008 Bioinformatics 24:2172-2176).

The signal peptide may be present at the N-terminus of the polypeptide, and may be present in the newly synthesised polypeptide. The signal peptide provides for efficient trafficking of the polypeptide. Signal peptides are often removed by cleavage, and thus are not comprised in the mature polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8:785-786) or Signal-BLAST (Frank and Sippl, 2008 Bioinformatics 24:2172-2176).

### Labels and Conjugates

In some embodiments, the antigen-binding molecules of the present disclosure and their constituent polypeptides comprise a detectable moiety.

In some embodiments, a detectable moiety is a fluorescent label, phosphorescent label, luminescent label, immuno-detectable label (e.g. an epitope tag), radiolabel, chemical, nucleic acid or enzymatic label. An antigen-binding molecule or a constituent polypeptide thereof may be covalently or non-covalently labelled with the detectable moiety.

Fluorescent labels include e.g. fluorescein, rhodamine, allophycocyanin, eosine and NDB, green fluorescent protein (GFP), chelates of rare earths such as europium (Eu), terbium (Tb) and samarium (Sm), tetramethyl rhodamine, Texas Red, 4-methyl umbelliferone, 7-amino-4-methyl coumarin, Cy3, and Cy5. Radiolabels include radioisotopes such as Hydrogen³, Sulfur³⁵, Carbon¹⁴, Phosphorus³², Iodine¹²³ Iodine¹²⁵, Iodine¹²⁶, Iodine¹³¹, Iodine¹³³, Bromine⁷⁷, Technetium⁹⁹m, Indium¹¹¹, Indium^{113m}, Gallium⁶⁷, Gallium⁶⁸, Ruthenium⁹⁵, Ruthenium⁹⁷, Ruthenium¹⁰³, Ruthenium¹⁰⁵, Mercury²⁰⁷, Mercury²⁰³, Rhenium⁹⁹m Rhenium¹⁰¹, Rhenium¹⁰⁵, Scandium⁴⁷, Tellurium¹²¹m, Tellurium¹²²m, Tellurium¹²⁵m, Thulium¹⁶⁵, Thuliuml¹⁶⁷, Thulium¹⁶⁸, Copper⁶⁷, Fluorine¹⁸, Yttrium⁹⁰, Palladium¹⁰⁰, Bismuth²¹⁷ and Antimony²¹¹. Luminescent labels include as radioluminescent, chemiluminescent (e.g. acridinium ester, luminol, isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers.

In some embodiments, an antigen-binding molecule or a constituent polypeptide thereof comprises a radionuclide. Such conjugates may be called radionuclide drug conjugates (RDCs) or radioimmunoconjugates (RICs). In some embodiments, an antigen-binding molecule or a constituent polypeptide thereof comprises a chelator group that is capable of chelating a radionuclide. Radionuclides include radioisotopes such as those listed above, or others such as Lutetium¹⁷⁷, Actinium²²⁵ and Strontium⁹⁰.

In some embodiments, an antigen-binding molecule or a constituent polypeptide thereof comprises an epitope tag, e.g. a His, (e.g. 6XHis), FLAG, c-Myc, StrepTag, haemagglutinin, E, calmodulin-binding protein (CBP), glutathione-s-transferase (GST), maltose-binding protein (MBP), thioredoxin, S-peptide, T7 peptide, SH2 domain, avidin, streptavidin, and haptens (e.g. biotin, digoxigenin, dinitrophenol), optionally at the N- or C-terminus of the antigen-binding molecule/polypeptide.

In some embodiments, an antigen-binding molecule or a constituent polypeptide thereof polypeptide comprises a moiety having a detectable activity, e.g. an enzymatic moiety. Enzymatic moieties include e.g. luciferases, glucose oxidases, galactosidases (e.g. beta-galactosidase), glucorinidases, phosphatases (e.g. alkaline phosphatase), peroxidases (e.g. horseradish peroxidase) and cholinesterases.

### Nucleic Acids and Vectors

The present disclosure provides a nucleic acid, or a plurality of nucleic acids, encoding an antigen-binding molecule/antigen-binding polypeptide complex or a constituent polypeptide thereof according to the present disclosure. In some embodiments, the nucleic acid(s) comprise or consist of DNA and/or RNA.

An antigen-binding molecule/antigen-binding polypeptide complex or a constituent polypeptide thereof according to the present disclosure may be produced within a cell by translation of RNA encoding the polypeptide(s). An antigen-binding molecule/antigen-binding polypeptide complex or a constituent polypeptide thereof may be produced within a cell by transcription from nucleic acid encoding the polypeptide(s), and subsequent translation of the transcribed RNA.

In some embodiments, the nucleic acid(s) may be, or may be comprised/contained in, a vector, or a plurality of vectors. A ‘vector’ as used herein is a nucleic acid molecule used as a vehicle to transfer exogenous nucleic acid into a cell.

Accordingly, the present disclosure also provides a vector, or plurality of vectors, comprising the nucleic acid or plurality of nucleic acids according to the present disclosure. The vector may facilitate delivery of the nucleic acid(s) encoding a polypeptide according to the present disclosure to a cell. The vector may be an expression vector comprising elements required for expressing a polypeptide according to the present disclosure. The vector may comprise elements facilitating integration of the nucleic acid(s) into the genomic DNA of cell into which the vector is introduced.

Nucleic acids and vectors according to the present disclosure may be provided in purified or isolated form, i.e. from other nucleic acid, or naturally-occurring biological material.

A vector may be a vector for expression of the nucleic acid in the cell (i.e. an expression vector). Such vectors may include a promoter sequence operably linked to a nucleotide sequence encoding an antigen-binding molecule or polypeptide according to the present disclosure. A vector may also include a termination codon (i.e. 3′ in the nucleotide sequence of the vector to the nucleotide sequence encoding the polypeptide(s)) and expression enhancers. Any suitable vectors, promoters, enhancers and termination codons known in the art may be used to express a peptide or polypeptide from a vector according to the present disclosure.

The term ‘operably linked’ may include the situation where nucleic acid encoding a polypeptide according to the present disclosure and regulatory nucleic acid sequence(s) (e.g. a promoter and/or enhancers) are covalently linked in such a way as to place the expression of the nucleic acid encoding a polypeptide under the influence or control of the regulatory nucleic acid sequence(s) (thereby forming an expression cassette). Thus, a regulatory sequence is operably linked to the selected nucleic acid sequence if the regulatory sequence is capable of effecting transcription of the nucleic acid sequence. The resulting transcript(s) may then be translated into the desired polypeptide(s).

Vectors contemplated in connection with the present disclosure include DNA vectors, RNA vectors, plasmids (e.g. conjugative plasmids (e.g. F plasmids), non-conjugative plasmids, R plasmids, col plasmids, episomes), viral vectors (e.g. retroviral vectors, e.g. gammaretroviral vectors (e.g. murine Leukemia virus (MLV)-derived vectors, e.g. SFG vector), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 and Morgan and Boyerinas, Biomedicines (2016) 4:9, which are both hereby incorporated by reference in their entirety. In some embodiments, a vector according to the present disclosure is a lentiviral vector.

In some embodiments, the vector may be a eukaryotic vector, i.e. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

Constituent polypeptides of an antigen-binding molecule/antigen-binding polypeptide complex according to the present disclosure may be encoded by different nucleic acids of the plurality of nucleic acids, or by different vectors of the plurality of vectors.

### Producing the Antigen-Binding Molecules

Antigen-binding molecules according to the present disclosure may be prepared according to methods for the production of antibody-drug conjugates known to the skilled person.

Antigen-binding moieties according to the present disclosure may be prepared by chemical synthesis, e.g. liquid or solid phase synthesis. For example, peptides/polypeptides can be synthesised using the methods described in, for example, Chandrudu et al., Molecules (2013), 18:4373-4388, which is hereby incorporated by reference in its entirety.

Alternatively, antigen-binding moieties according to the present disclosure may be produced by recombinant expression. Molecular biology techniques suitable for recombinant production of polypeptides are well known in the art, such as those set out in Green and Sambrook, Molecular Cloning: A Laboratory Manual (4^{th} Edition), Cold Spring Harbor Press, 2012, and in Nat Methods. (2008); 5(2): 135-146 both of which are hereby incorporated by reference in their entirety. Methods for the recombinant production of antigen-binding polypeptides are also described in Frenzel et al., Front Immunol. (2013); 4: 217 and Kunert and Reinhart, Appl Microbiol Biotechnol. (2016) 100:3451-3461, both of which are hereby incorporated by reference in their entirety.

In some cases, the antigen-binding moieties of the present disclosure are comprised of more than one polypeptide chain. In such cases, production of the antigen-binding moiety may comprise transcription and translation of more than one polypeptide, and subsequent association of the polypeptide chains to form the antigen-binding moiety.

For recombinant production according to the present disclosure, any cell suitable for the expression of polypeptides may be used. The cell may be a prokaryote or eukaryote. In some embodiments, the cell is a prokaryotic cell, such as a cell of archaea or bacteria. In some embodiments, the bacteria may be Gram-negative bacteria such as bacteria of the family Enterobacteriaceae, for example Escherichia coli. In some embodiments, the cell is a eukaryotic cell such as a yeast cell, a plant cell, insect cell or a mammalian cell, e.g. a cell described hereinabove. In some cases, the cell is not a prokaryotic cell because some prokaryotic cells do not allow for the same folding or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

In some embodiments polypeptides may be prepared by cell-free-protein synthesis (CFPS), e.g. according to a system described in Zemella et al. Chembiochem (2015) 16 (17): 2420-2431, which is hereby incorporated by reference in its entirety.

Production of antigen-binding moieties may involve culture or fermentation of a eukaryotic cell modified to express the polypeptide(s) of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide(s). Culture, fermentation and separation techniques are well known to those of skill in the art, and are described, for example, in Green and Sambrook, Molecular Cloning: A Laboratory Manual (4^{th} Edition; incorporated by reference herein above). Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the polypeptide(s), the polypeptide(s) of interest may be isolated. Any suitable method for separating proteins from cells known in the art may be used. In order to isolate the polypeptide, it may be necessary to separate the cells from nutrient medium. If the polypeptide(s) are secreted from the cells, the cells may be separated by centrifugation from the culture media that contains the secreted polypeptide(s) of interest. If the polypeptide(s) of interest collect within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polypeptide(s) of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins. Other methods for distinguishing different proteins are known in the art, for example ion exchange chromatography and size chromatography. These may be used as an alternative to precipitation or may be performed subsequently to precipitation.

Once the polypeptide(s) of interest have been isolated from culture it may be desired or necessary to concentrate the polypeptide(s). A number of methods for concentrating proteins are known in the art, such as ultrafiltration or lyophilisation.

Antigen-binding polypeptides/polypeptide complexes according to the present disclosure may be conjugated to linker-payload moieties according to the present disclosure for the production of antigen-binding molecules according to the present disclosure by any suitable techniques, which are well known to the skilled person and routinely employed in the art.

General methods for the conjugation of antigen-binding polypeptides/polypeptide complexes to linker-payload moieties are described e.g. in Chudasama et al., Nature Chemistry, (2016), 8:114-119, Baah et al., Molecules. (2021) 26 (10): 2943, and Walsh et al., Chem. Soc. Rev. (2021) 50:1305-1353, all of which are hereby incorporated by reference in their entirety. Conjugation of antigen-binding moieties and linker-payload moieties and purification of antigen-binding molecules produced by such conjugation is described e.g. in Beck et al., (2017) Nat Rev Drug Discov 16:315-337; Peters and Brown Biosci Rep (2015) 35: art:e00225; McCombs and Owen, The AAPS Journal (2015) 17:339-351; Jackson, Org Process Res Dev (2016) 20:852-866; and Olivier and Hurvitz, Antibody-Drug Conjugates: Fundamentals, Drug Development, and Clinical, (2016) Wiley.

General methods for the conjugation of antigen-binding polypeptides/polypeptide complexes to linker-payload moieties are described e.g. in Chudasama et al., Nature Chemistry, (2016), 8:114-119, Baah et al., Molecules. (2021) 26 (10): 2943, and Walsh et al., Chem. Soc. Rev. (2021) 50:1305-1353, all of which are hereby incorporated by reference in their entirety. Conjugation of antigen-binding moieties and linker-payload moieties and purification of antigen-binding molecules produced by such conjugation is described e.g. in Beck et al., (2017) Nat Rev Drug Discov 16:315-337; Peters and Brown Biosci Rep (2015) 35: art:e00225; McCombs and Owen, The AAPS Journal (2015) 17:339-351; Jackson, Org Process Res Dev (2016) 20:852-866; and Olivier and Hurvitz, Antibody-Drug Conjugates: Fundamentals, Drug Development, and Clinical Outcome to Target Cancer (2016) Wiley.

Antigen-binding moieties according to the present disclosure may be conjugated to linker-payload moieties according to the present disclosure by any suitable techniques, which are well known to the skilled person and routinely employed in the art. Such methods are described e.g. in Chudasama et al., Nature Chemistry, (2016), 8:114-119, Baah et al., Molecules. (2021) 26 (10): 2943, and Walsh et al., Chem. Soc. Rev. (2021) 50:1305-1353, all of which are hereby incorporated by reference in their entirety.

Conjugation of antigen-binding moieties and linker-payload moieties and the purification of antigen-binding molecules produced by such conjugation can be performed e.g. as described in in Beck et al., (2017) Nat Rev Drug Discov 16:315-337; Peters and Brown Biosci Rep (2015) 35: art:e00225; McCombs and Owen, The AAPS Journal (2015) 17:339-351; Jackson, Org Process Res Dev (2016) 20:852-866; or Olivier and Hurvitz, Antibody-Drug Conjugates: Fundamentals, Drug Development, and Clinical Outcome to Target Cancer, (2016) Wiley, all of which are hereby incorporated by reference in their entirety. Other relevant disclosures relating to conjugation and linkers are: Tsuchikama and An, Protein Cell. (2018) 9 (1): 33-46, Khongorzul et al., Mol Cancer Res (2020) 18 (1): 3-19 and Drago et al., Nature Reviews Clinical Oncology (2021) 18:327-344, all of which are hereby incorporated by reference in their entirety.

### Lysine Amide Coupling

Lysine-based conjugation is one of the most widely used non-specific conjugation strategies. Such conjugation occurs on reactive amine side chains of lysine residues due to their good nucleophilicity. Immunoglobulin scaffolds contains over 80 lysine residues, most of which are exposed on the surface of the molecule. Among the surface lysine residues, more than 20 have been shown as highly solvent-accessible and can serve as potential ADC conjugation sites. Lysine conjugation follows two main strategies that result in the formation of a stable amide or amidine bond between the protein and the drug-linker complex. Generally speaking, activated esters on the drug-linker complexes, often O-succinimide reagents such as N-hydroxysuccinimidyl (NHS) or sulfo-NHS esters, react with the antibody lysine residues and achieve the conjugation via amide bonds. On the other hand, stable amidine bonds can be generated on an antibody by the reaction of imido ester compounds, such as Traut's reagent, with antibody lysine residues.

A one-step conjugation of a drug-linker moiety containing an amine-reactive group to the antibody via amide bonds is known, as well as two-step conjugation, where in the first step, a small bi-functional reagent containing both an amine- and a thiol-reactive functional groups is reacted with the available lysine ε-amino groups to serve as a chemical adaptor, leaving free thiol-reactive groups on the antibody. In the second step, the payload drugs or drug-linker complexes are attached to the thiol-reactive groups introduced previously to form the ADC. The two-step approach is often used when the drug/drug-linker complex contains a thiol-reactive module or as an alternative route when introducing an amine-reactive module into the drug or drug-linker complex is proven to be difficult. Four small adaptors commonly used in the two-step conjugation: SPDB disulfide, MCC (maleimidomethyl cyclohexane-1-carboxylate), sulfo-SPDB, and Hydrazine.

### Cysteine Coupling

Cysteine modification occurs most commonly by 1,4-conjugate addition to N-substituted maleimides. Maleimides are particularly attractive reagents due to their synthetic accessibility and rapid reaction rates with cysteine under mild conditions. The resulting thiosuccinimide conjugates are inherently unstable, due to their propensity towards retro-Michael addition. This instability can be mitigated by forcing post-conjugation hydrolysis of the thiosuccinimide, creating a stable chemical linkage. Accordingly, a number of “self-hydrolysing” maleimides have now been developed, with ring-opening catalysed by adjacent functional groups such as primary amine, polyethylene glycol (PEG) and N-aryl amongst the most promising. Other reagents including α-halocarbonyls, palladium oxidative-addition complexes, ethynylphosphonamidates, vinylphosphonites and ethynylbenziodoxolones.

Some non-maleimide cysteine conjugations are summarised in Kang, et al., Chem Sci (2021) 12, 13613-13647 (doi: 10.1039/D1SC02973H), and include the use of:
(i) alkynyl carboxylic acid derivatives;
(ii) 5-methylene pyrrolone (5MP);
(iii) 5,5′-dithiobis-(2-nitrobenzoic acid) (DTNB);
(iv) phenyloxadiazole sulfone (PODS);
(v) aza-dibenzocyclooctyne (DBCO);
(vi) phosphonamidite;
(vii) 3-arylpropionitrile (APN);
(viii) perfluoroarene;
(ix) ethynylbenziodoxolone (EBX);
(x) bicyclo[1.1.0]butane (BCB) carboxylic amide; and
(xi) allenamide.

Another possible approach is described in Cheng, et al., Front. Oncol. 12:951589 (doi: 10.3389/fonc.2022.951589) where 2-methylsulfonyl pyrimidine is used instead of a maleimide.

Genetic modification of the number of accessible cysteine residues on an antibody surface is a method to achieve site-selective and homogeneous modification. For example, in THIOMABs, the engineered cysteine is installed on an anti-MUC16 antibody by mutation of heavy chain alanine 114 (HC-A114). Other approaches have engineered antibodies to contain cysteine mutations at D265C, S239C, E269C, K326C or A327C, or to insert additional cysteines before and after positions HC-S239, HC-A114, and LC-V205.

### Non-Natural Amino Acid Incorporation by Genetic Engineering

Site-specific incorporation of non-canonical amino acids (ncAAs) into antibodies results in an efficient approach to the site-specific modification of antibodies, and therefore homogeneous ADCs.

NcAAs bearing unique functionalities, such as ketones, azides, cyclopropenes or diene functional groups, have been developed and incorporated into antibodies. Such ncAAs include p-acetylphenylalanine (pAcF), which has a ketone side chain which can participate in oxime ligation reactions; NE-(1-methylcycloprop-2-enecarboxamido)-lysine (CpK), which has a cyclopropene side chain which can participate in IEDDA reactions; para-azidomethyl phenylalanine (pAMF), which has a an azide side chain which can undergo click reactions; spiro[2.4]hepta-4,6-diene-lysine (SCpHK), which has a spiro[2.4]hepta-4,6-diene side chain which can participate in Diels-Alder reactions; and N6-(2-azidoethoxy)-carbonyl-L-lysine (AzK), which has a an azide side chain which can undergo click reactions.

Azide-containing ncAAs can undergo rapid CuAAC or SPAAC reactions under physiological conditions. para-azidophenylalanine (pAzF) can undergo reactions with, for example, cyclooctyne-functionalised linkers and dibenzylcyclooctyne (DBCO)-functionalised linkers. A cyclopropene derivative of lysine (N ϵ-[((2-methylcycloprop-2-en-1-yl) methoxy) carbonyl]-l-lysine; CypK) can undergo a rapid and efficient inverse-electron demand Diels-Alder (IEDDA) reaction with a tetrazine-functionalised linker.

Cyclopentadiene-containing ncAAs, spiro[2.4]hepta-4,6-diene-lysine (SCpHK) and cyclopentadiene-lysine (CpHK), can undergo irreversible Diels-Alder cycloadditions with maleimide-modified drugs.

### Enzymatic Conjugation

Enzymes can be used to achieve site-selective antibody modification due to their high specificity and mild reaction conditions. Enzymes can either directly attach a payload to a specific amino acid sequence or introduce a reactive functionality on the antibody that can be further functionalised with the desired payload.

### Transpeptidation Using Sortase

Sortase-mediated antibody conjugation (SMAC) technology is an additional enzymatic ligation approach. SMAC-technology uses S. aureus sortase A, which is a transpeptidase that cleaves the amide bond between threonine and glycine residues in the LPXTG (X=any amino acid) pentapeptide motif, and subsequently catalyses the attachment of glycine-functionalised payloads to the newly generated C-terminus. The sortase recognition motif and a Strep II tag, which is used to aid removal of unreacted antibody, were fused to the light and heavy chain C-terminus of different antibodies. Sortase-mediated conjugation can then be used to attach a series of penta-glycine tagged payloads.

### Transpeptidation Using Microbial Transglutaminase

The use of bacterial transglutaminases is a powerful approach for site-specific incorporation of the payload into the antibody. A transglutaminase derived from Streptomyces mobaraensis catalyzes transpeptidation where a primary amine-containing linker is covalently attached to the primary amide side chain of a specific glutamine (Q295) within deglycosylated antibodies, resulting in ADCs with a defined DAR arising from the conjugation of 2 linker-payloads (one conjugation site per heavy chain). An N297Q mutation prior to this conjugation provides two more reaction sites (resulting in the conjugation of 4-linker-payloads). An alternative version using a peptide sequence-specific transglutaminase. This enzyme recognizes and utilizes LLQG motif that is genetically incorporated, resulting in site-specific antibody-drug conjugation. Another advantage of this LLQG-specific bacterial transglutaminase is that conjugation sites can be flexibly laid by inserting this short peptide motif within the antibody structure. Further alternative approaches allow for the use of transglutaminase without deglycosylation.

The DAR will depend on the number of payloads per linker-payload moieties conjugated.

### N-Glycan Engineering

Asn297 (N297) within the Fc domain and the N-glycan on this residue are conserved in all IgG classes, making these components attractive reaction sites for broadly applicable ADC conjugation. Incorporation of an aldehyde group on the N-glycan terminus using β-1,4-galactosyltransferase (GalT) and α-2,6-sialyltransferase (SialT) introduce a sialic acid on each N-glycan terminus, which is subsequently converted into an aldehyde group using NalO₄ under mild oxidation conditions. The aldehyde groups generated can then used to conjugate aminooxyfunctionalized payloads.

Another approach is to incorporate non-natural saccharides possessing orthogonal reaction handles into the antibody. A technology based on this strategy is the GlycoConnect in which the glycan chain at Asn297 is trimmed using the endoglycosidase Endo S2 and then azide groups are introduced using a mutant galactosyl transferase GalT (Y289L) and N-azidoacetylgalactosamine (GaINAz). The azide handles can be used for a strain-promoted click reaction with payloads.

In some embodiments, the linker-payload terminates in an amino group which is conjugated to the antigen-binding molecule using transglutaminase.

In some embodiments, the method further comprises purifying/isolating the antigen-binding molecule (i.e. from unreacted precursors and/or by-products). In some embodiments, the antigen-binding molecule may be purified/isolated by chromatography, e.g. size-exclusion chromatography.

The present disclosure also provides an antigen-binding molecule obtained or obtainable by the methods of the present disclosure.

### Compositions

The present disclosure provides a composition comprising an antigen-binding molecule according to the present disclosure.

The antigen-binding molecules described herein may be formulated as pharmaceutical compositions or medicaments for clinical use and may comprise a pharmaceutically-acceptable carrier, diluent, excipient or adjuvant. Thus, the present disclosure provides a pharmaceutical composition/medicament comprising an antigen-binding molecule described herein.

The pharmaceutical compositions/medicaments of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilisers, solubilisers, surfactants (e.g., wetting agents), masking agents or colouring agents (e.g. titanium oxide).

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabiliser, solubiliser, surfactant, masking agent, colouring agent, flavouring agent or sweetening agent of a composition according to the present disclosure must also be ‘acceptable’ in the sense of being compatible with the other ingredients of the formulation. Suitable carriers, diluents, excipients, adjuvants, fillers, buffers, preservatives, anti-oxidants, lubricants, binders, stabilisers, solubilisers, surfactants, masking agents, colouring agents, flavouring agents or sweetening agents can be found in standard pharmaceutical texts, for example, Remington's ‘The Science and Practice of Pharmacy’ (Ed. A. Adejare), 23^{rd} Edition (2020), Academic Press.

Pharmaceutical compositions and medicaments of the present disclosure may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration. In some embodiments, a pharmaceutical composition/medicament may be formulated for administration by injection or infusion, or administration by ingestion.

Suitable formulations may comprise the antigen-binding molecule provided in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments, the pharmaceutical compositions/medicament is formulated for injection or infusion, e.g. into a blood vessel, tissue/organ of interest, or a tumor.

The present disclosure also provides methods for the production of pharmaceutically useful compositions, such methods of production may comprise one or more steps selected from:
producing an antigen-binding molecule described herein;
isolating/purifying an antigen-binding molecule described herein; and/or
mixing an antigen-binding molecule described herein with a pharmaceutically-acceptable carrier, adjuvant, excipient or diluent.

For example, a further aspect of the present disclosure relates to a method of formulating or producing a medicament or pharmaceutical composition for use in the treatment of a disease/condition (e.g. a disease/condition described herein), the method comprising formulating a pharmaceutical composition or medicament by mixing an antigen-binding molecule described herein with a pharmaceutically-acceptable carrier, adjuvant, excipient or diluent.

### Therapeutic and Prophylactic Applications

The antigen-binding molecules and compositions described herein find use in therapeutic and prophylactic intervention for disease, e.g. cancers.

It will be appreciated that the antigen-binding molecules and compositions of the present disclosure may be used for the treatment/prevention of any disease/condition that would derive therapeutic or prophylactic benefit from a reduction in the level of expression or activity of TROP2, or a reduction in the number or activity of cells comprising/expressing TROP2.

For example, the disease/condition may be a disease/condition in which TROP2, or cells expressing/overexpressing TROP2 are pathologically-implicated, e.g. a disease/condition in which an increased level/activity of TROP2, or an increase in the number/proportion of cells comprising/expressing TROP2 is positively associated with the onset, development or progression of the disease/condition, and/or severity of one or more symptoms of the disease/condition. In some embodiments, an increased level/activity of TROP2, or an increase in the number/proportion of cells comprising/expressing TROP2 may be a risk factor for the onset, development or progression of the disease/condition.

The present disclosure provides an antigen-binding molecule or composition described herein for use in a method of medical treatment or prophylaxis. Also provided is an antigen-binding molecule or composition described herein for use in a method of treating or preventing a cancer (e.g. a cancer described herein). Also provided is the use of an antigen-binding molecule or composition described herein in the manufacture of a medicament for treating or preventing a cancer (e.g. a cancer described herein). Also provided is a method of treating or preventing a cancer (e.g. a cancer described herein) in a subject, comprising administering to a subject a therapeutically or prophylactically effective amount of an antigen-binding molecule or composition described herein.

The methods may be effective to reduce the development or progression of a cancer, alleviation of the symptoms of a cancer or reduction in the pathology of a cancer. The methods may be effective to prevent progression of the cancer, e.g. to prevent worsening of, or to slow the rate of development of, the cancer. In some embodiments, the methods may lead to an improvement in the cancer, e.g. a reduction in the symptoms of the cancer or reduction in some other correlate of the severity/activity of the cancer. In some embodiments, the methods may prevent development of the cancer to a later stage (e.g. a chronic stage or metastasis).

As used herein, a ‘cancer’ may be or comprise any unwanted cell proliferation (or any disease manifesting itself by unwanted cell proliferation), neoplasm or tumor. The cancer may be benign or malignant. The cancer may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, biliary tract, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue; examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, hematologic cancer and sarcoma.

In some embodiments the cancer is a cancer in which TROP2 is pathologically-implicated. That is, in some embodiments the cancer is a cancer which is caused or exacerbated by the expression of TROP2, a cancer for which expression of TROP2 is a risk factor and/or a cancer for which expression of TROP2 is positively associated with onset, development, progression, severity or metastasis of the cancer. The cancer may be characterised by expression of TROP2, e.g. the cancer may comprise cells (e.g. cells of tumor tissue) expressing TROP2. Such cancers may be referred to as being positive for TROP2. A cancer which is ‘positive’ for TROP2 may be a cancer comprising cells expressing TROP2 (e.g. at the cell surface). A cancer which is ‘positive’ for TROP2 may overexpress TROP2.

Expression may be determined by any suitable means. Expression may be gene expression or protein expression. Gene expression can be determined e.g. by detection of mRNA encoding TROP2, for example by quantitative real-time PCR (qRT-PCR). Protein expression can be determined e.g. by antibody-based methods, for example by western blot, immunohistochemistry, immunocytochemistry, flow cytometry, or ELISA.

In some embodiments, the cancer to be treated/prevented comprises cells harboring a genetic variant (e.g. a mutation) which causes increased (gene and/or protein) expression and/or activity of TROP2, relative to comparable cells harboring a reference allele not comprising the genetic variant (e.g. a non-mutated, or ‘wildtype’ allele). The genetic variant may be or comprise insertion, deletion, substitution to, or larger-scale translocation/rearrangement of, the nucleotide sequence relative to the reference allele.

A mutation ‘resulting in’ increased expression of TROP2 may be known or predicted to cause, or may be associated with, increased gene/protein expression of TROP2. A mutation ‘resulting in’ increased activity of TROP2 may be known or predicted to cause, or may be associated with, increased TROP2-mediated signaling. Mutations resulting in increased expression and/or activity of TROP2 may be referred to as ‘activating’ mutations.

A mutation which causes increased expression of TROP2 may result in gene or protein expression of TROP2 which is not expressed by, and/or not encoded by genomic nucleic acid of, an equivalent cell not harboring the mutation. That is, the expression of TROP2 may be a result of the mutation, and thus ‘increased expression’ may be from no expression.

A mutation which causes increased expression of TROP2 may result in increased gene or protein expression of TROP2 which is expressed by, and/or which is encoded by genomic nucleic acid of, an equivalent cell not comprising the mutation. By way of illustration, a cell may comprise a mutation resulting in an increase in the level of transcription of nucleic acid encoding TROP2 relative to the level of transcription of nucleic acid encoding TROP2 by an equivalent cell not comprising the mutation.

In some embodiments, a mutation which causes increased expression of TROP2 may cause an increase in gene expression of TROP2 relative to an equivalent cell not comprising the mutation. In some embodiments, a mutation which causes increased expression of TROP2 may cause an increase in protein expression of TROP2 relative to an equivalent cell not comprising the mutation.

In some embodiments, a mutation which causes increased expression of TROP2 may cause an increase in the level of TROP2 on or at the cell surface of a cell comprising the mutation, relative to an equivalent cell not comprising the mutation.

Cells having increased expression of TROP2 relative to the level of expression of TROP2 by a reference cell (e.g. as a result of mutation) may be described as ‘overexpressing’ TROP2, or having ‘upregulated expression’ of TROP2. For example, a cancer comprising cells harboring a mutation resulting in increased expression of TROP2 relative to equivalent cells lacking the mutation may be described as a cancer comprising cells displaying overexpression/upregulated expression of TROP2. In some embodiments, the reference cell lacking the mutation may be a non-cancerous cell (e.g. of equivalent cell type) or a cancerous cell (e.g. of equivalent cancer type).

A mutation which causes increased activity of TROP2 may result in an increase in TROP2-mediated signaling relative to the level of TROP2-mediated signaling by an equivalent cell not comprising the mutation.

It will be appreciated that a mutation which causes increased expression/activity of TROP2 may be a mutation which impacts a pathway associated with the regulation of TROP2 expression/activity. That is, a mutation may cause increased or decreased expression/activity of a component/molecule/protein/nucleic acid of a pathway that is associated with regulating TROP2 expression/activity, and this may result in increased expression/activity of TROP2.

In some embodiments, a cancer to be treated/prevented in accordance with the present disclosure may be characterised by an increase in the expression and/or activity of TROP2 (i.e. gene and/or protein expression) in an organ/tissue/subject affected by the disease/condition e.g. as compared to normal organ/tissue/subject (i.e. in the absence of the disease/condition). In some embodiments, cells and/or a tumor of a cancer to be treated/prevented may be characterised by an increase in the expression and/or activity of TROP2, e.g. as compared to the level of expression and/or activity observed in equivalent non-cancerous cells/non-tumor tissue.

TROP2 and its association with and role in cancer is reviewed e.g. in Qiu et al., BBA—Reviews on Cancer (2023) 1878:188902, Chmielewska & Kubiatowski, Oncology in Clinical Practice (2023) 19(6): 427-432, and Lombardi et al., Cancers (2023) 15 (6): 1744, all of which are hereby incorporated by reference in their entirety.

Overexpression of TROP2 has been reported in a wide range of cancers. Tumor types with strong to moderate TROP2 IHC staining include: lung cancers (e.g. non-small cell lung cancers (e.g. adNSCLC, sqNSCLC)), breast cancers (e.g. hormone receptor-positive (HR+) breast cancer, metastatic breast cancer, HR+ metastatic breast cancer, HER2-positive (HER2+) breast cancer, HER2+ metastatic breast cancer, triple-negative breast cancer (TNBC)), pancreatic cancers, prostate cancers (e.g. castration-resistant prostate cancer (CRPC), metastatic castration-resistant prostate cancer (mCRPC)), bladder cancers, head and neck cancers (e.g. head and neck squamous cell carcinomas (HNSCC)), endometrial cancers, ovarian cancers, esophageal cancers (e.g. squamous-cell carcinoma (ESCC)), cervical cancers, thyroid cancers and biliary tract cancers (BTC). Expression of TROP2 in tumors and normal tissue is described in e.g. Dum et al., Pathobiology (2022) 89 (4): 245-258. Accordingly, in some embodiments, the cancer to be treated is selected from: lung cancer (e.g. non-small cell lung cancer (e.g. adNSCLC, sqNSCLC)), breast cancer (e.g. hormone receptor-positive (HR+) breast cancer, metastatic breast cancer, HR+ metastatic breast cancer, HER2-positive (HER2+) breast cancer, HER2+ metastatic breast cancer, triple-negative breast cancer (TNBC)), pancreatic cancer, prostate cancer (e.g. castration-resistant prostate cancer (CRPC), metastatic castration-resistant prostate cancer (mCRPC)), bladder cancer, head and neck cancer (e.g. head and neck squamous cell carcinoma (HNSCC)), endometrial cancer, ovarian cancer, esophageal cancer (e.g. esophageal squamous-cell carcinoma (ESCC)), cervical cancer, thyroid cancer, and biliary tract cancer (BTC). In some embodiments, the cancer to be treated is selected from: non-small cell lung cancer adenocarcinoma (adNSCLC), hormone receptor-positive breast cancer (HR+BC), metastatic castration-resistant prostate cancer (mCRPC), bladder cancer, endometrial cancer, triple-negative breast cancer (TNBC), ovarian cancer, pancreatic cancer, head and neck squamous cell carcinoma (HNSCC), and esophageal squamous-cell carcinoma (ESCC).

In some embodiments, a cancer according to the present disclosure is a cancer for which sacituzumab govitecan is an approved treatment. In some embodiments, a cancer is selected from: triple-negative breast cancer (e.g. metastatic or unresectable triple negative breast cancer); hormone receptor-positive (i.e. a cancer comprising cells expressing estrogen receptor (ER) and/or progesterone receptor (PR)) and HER2-negative breast cancer (e.g. metastatic or unresectable hormone receptor-positive and HER2-negative breast cancer); bladder cancer and cancers of the urinary tract (e.g. urothelial cancer (e.g. metastatic or unresectable urothelial cancer)).

In some embodiments, the cancer to be treated is selected from: a cancer comprising cells expressing/overexpressing TROP2, a solid tumour, metastatic cancer, unresectable cancer, hormone receptor-positive cancer, lung cancer, non-small cell lung cancer (NSCLC), non-small cell lung cancer adenocarcinoma (adNSCLC), squamous non-small cell lung cancer (sqNSCLC), breast cancer, hormone receptor-positive breast cancer, metastatic breast cancer, hormone receptor-positive metastatic breast cancer, HER2-positive breast cancer, HER2-positive metastatic breast cancer, HER2-negative breast cancer, metastatic HER2-negative breast cancer, unresectable HER2-negative breast cancer, hormone receptor-positive and HER2-negative breast cancer, metastatic hormone receptor-positive and HER2-negative breast cancer, unresectable hormone receptor-positive and HER2-negative breast cancer, triple-negative breast cancer (TNBC), metastatic triple negative breast cancer, unresectable triple-negative breast cancer, pancreatic cancer, prostate cancer, castration-resistant prostate cancer (CRPC), metastatic castration-resistant prostate cancer (mCRPC)), bladder cancer, head and neck cancer, head and neck squamous cell carcinoma (HNSCC), endometrial cancer, ovarian cancer, esophageal cancer, esophageal squamous-cell carcinoma (ESCC), cervical cancer, thyroid cancer, biliary tract cancer (BTC), urinary tract cancer, urothelial cancer, metastatic urothelial cancer, and unresectable urothelial cancer.

In some embodiments, a cancer to be treated/prevented in accordance with the present disclosure comprises/expresses/overexpresses canonical/wild-type TROP2 (e.g. TROP2 having the amino acid sequence as shown in SEQ ID NO: 1 or 2). In some embodiments, a cancer to be treated/prevented in accordance with the present disclosure does not comprise/express/overexpress TROP2 comprising a mutation (e.g. a mutation that attenuates/decreases the binding of the antigen-binding moiety to TROP2, or a mutation that reduces TROP2 plasma membrane localisation). In some embodiments, a cancer to be treated/prevented in accordance with the present disclosure does not comprise/express/overexpress TROP2 comprising a T256R mutation (i.e. TROP2 comprising substitution of T at position 256 of SEQ ID NO: 1, or the position corresponding to position 256 of SEQ ID NO:1, with R).

In some embodiments, the cancer may be a relapsed cancer. As used herein, a ‘relapsed’ cancer refers to a cancer which responded to a treatment (e.g. a first line therapy for the cancer), but which has subsequently re-emerged/progressed, e.g. after a period of remission. For example, a relapsed cancer may be a cancer whose growth/progression was inhibited by a treatment (e.g. a first line therapy for the cancer), and which has subsequently grown/progressed. A cancer that is relapsed with respect to given treatment may be described as having acquired resistance to such treatment.

In some embodiments, the cancer may be a refractory cancer. As used herein, a ‘refractory’ cancer refers to a cancer which has not responded to a treatment (e.g. a first line therapy for the cancer). For example, a refractory cancer may be a cancer whose growth/progression was not inhibited by a treatment (e.g. a first line therapy for the cancer). In some embodiments a refractory cancer may be a cancer for which a subject receiving treatment for the cancer did not display a partial or complete response to the treatment. A cancer that is refractory with respect to given treatment may be described as having intrinsic resistance to such treatment.

In some embodiments, the cancer is a cancer that is relapsed or refractory with respect to treatment with a DNA damage response (DDR) inhibitor. In some embodiments, the cancer is refractory with respect to treatment with a DDR inhibitor. In some embodiments, the cancer is relapsed with respect to treatment with a DDR inhibitor. In some embodiments, the cancer has intrinsic resistance to treatment with a DDR inhibitor. In some embodiments, the cancer has acquired resistance to treatment with a DDR inhibitor. In accordance with such embodiments, the DDR inhibitor may have been administered in the form of an antigen-binding molecule comprising a payload moiety comprising or consisting of the DDR inhibitor, or may have been administered in unconjugated form.

Herein, where a cancer is described as being relapsed/refractory/resistant, etc. with respect to a given intervention, it may be simply described as being ‘relapsed/refractory/resistant to’ the relevant intervention.

DDR inhibitors and their use for the treatment of cancers is described e.g. in Cheng et al., Eur J Med Chem. (2022) 230:114109, Wang et al., Front Immunol. (2022) 13:854730 and Choi and Lee, Int J Mol Sci. (2022) 23 (3): 1701, all of which are hereby incorporated by reference in their entirety. In some embodiments, a DDR inhibitor according to the present disclosure is selected from: a PARP inhibitor (e.g. olaparib, rucaparib, niraparib, talazoparib, veliparib, pamiparib, simmiparib, senaparib, SC-10914, 2X-121, AMXI-5001, JPI-547, AZD5305, IDX-1197, TQB-3823, HWH-340, AsiDNA, STP-1002, RBN-2397), an ATM inhibitor (e.g. CP-466722, KU-55933, KU-60019, KU-59403, AZ31, AZ32, AZD0156, AZD1390), an ATR inhibitor (e.g. M6620 (berzosertib), M4344 (VX-803), AZD6738 (ceralasertib), BAY1895344 (elimusertib)), a WEE1 inhibitor (e.g. adavosertib, Debio 0123, PD0166285, PD0407824, AZD1775), a CHK1/2 inhibitor (e.g. CBP-501, prexasertib, MK-8776, GDC-0575, SRA-737), a DNA-PK inhibitor (e.g. CC-115, LY-3023414, AsiDNA, M3814 (nedisertib), M9831 (VX-984)) and a PLK1 inhibitor (e.g. BI-6727 (volasertib), PCM-075 (onvansertib)).

In some embodiments, the cancer is a cancer that is relapsed or refractory with respect to treatment with a DNA topoisomerase I (TOP1) inhibitor. In some embodiments, the cancer is refractory with respect to treatment with a TOP1 inhibitor. In some embodiments, the cancer is relapsed with respect to treatment with a TOP1 inhibitor. In some embodiments, the cancer has intrinsic resistance to treatment with a TOP1 inhibitor. In some embodiments, the cancer has acquired resistance to treatment with a TOP1 inhibitor. In accordance with such embodiments, the TOP1 inhibitor may have been administered in the form of an antigen-binding molecule comprising a payload moiety comprising or consisting of the TOP1 inhibitor, or may have been administered in unconjugated form.

DNA topoisomerase I inhibitors and their use for the treatment of cancers is described e.g. in Pommier, Chem Rev. (2009) 109 (7): 2894-2902, Li et al., Am J Cancer Res. (2017) 7 (12): 2350-2394 and Thomas and Pommier, Clin Cancer Res. (2019) 25 (22): 6581-6589, all of which are hereby incorporated by reference in their entirety. In some embodiments, a TOP1 inhibitor according to the present disclosure is selected from: camptothecin, irinotecan, etirinotecan, SN-38, DX-8951f (extatecan mesylate), DXd (1), DXd (2), exatecan, FL118, topotecan, gimatecan, belotecan, deruxtecan, belotecan, rubitecan, lurtotecan, diflomotecan, karenitecan, silatecan, namitecan, elomotecan, DRF-1042, delimotecan, NSC606985, chimmitecan, ZBH-1205, Genz-644282, non-CPT1, indotecan (LMP-400), indimitecan (LMP-776) and LMP744.

In some embodiments, the cancer to be treated/prevented in accordance with the present disclosure is a cancer that is: relapsed or refractory with respect to treatment with a DDR inhibitor (e.g. a DDR inhibitor as described herein), and relapsed or refractory with respect to treatment with a TOP1 inhibitor (e.g. a TOP1 inhibitor as described herein). In some embodiments, the cancer is: refractory with respect to treatment with a DDR inhibitor (e.g. a DDR inhibitor as described herein), and refractory with respect to treatment with a TOP1 inhibitor (e.g. a TOP1 inhibitor as described herein). In some embodiments, the cancer is: relapsed with respect to treatment with a DDR inhibitor (e.g. a DDR inhibitor as described herein), and relapsed with respect to treatment with a TOP1 inhibitor (e.g. a TOP1 inhibitor as described herein). In some embodiments, the cancer is: refractory with respect to treatment with a DDR inhibitor (e.g. a DDR inhibitor as described herein), and relapsed with respect to treatment with a TOP1 inhibitor (e.g. a TOP1 inhibitor as described herein). In some embodiments, the cancer is: relapsed with respect to treatment with a DDR inhibitor (e.g. a DDR inhibitor as described herein), and refractory with respect to treatment with a TOP1 inhibitor (e.g. a TOP1 inhibitor as described herein). In accordance with such embodiments, the TOP1 inhibitor and/or DDR inhibitor may have been administered in the form of an antigen-binding molecule comprising a payload moiety comprising or consisting of the TOP1 inhibitor/DDR inhibitor, or may have been administered in unconjugated form.

Treatment of a cancer in accordance with the methods of the present disclosure achieves one or more of the following treatment effects: reduces the number of cancer cells in the subject, reduces the size of a cancerous tumor/lesion in the subject, inhibits (e.g. prevents or slows) growth of cancer cells in the subject, inhibits (e.g. prevents or slows) growth of a cancerous tumor/lesion in the subject, inhibits (e.g. prevents or slows) the development/progression of a cancer (e.g. to a later stage, or metastasis), reduces the severity of symptoms of a cancer in the subject, increases survival of the subject (e.g. progression free survival or overall survival), reduces a correlate of the number or activity of cancer cells in the subject, and/or reduces cancer burden in the subject.

Subjects may be evaluated in accordance with the Revised Criteria for Response Assessment: The Lugano Classification (described e.g. in Cheson et al., J Clin Oncol (2014) 32:3059-3068, incorporated by reference hereinabove) in order to determine their response to treatment. In some embodiments, treatment of a subject in accordance with the methods of the present disclosure achieves one of the following: complete response, partial response, or stable disease.

Prevention may refer to prevention of development of a cancer, and/or prevention of worsening of a cancer, e.g. prevention of progression of a cancer, e.g. to a later stage (e.g. metastasis).

In some embodiments, administration of an antigen-binding molecule/composition according to the present disclosure may be associated with one or more of: inhibition of the development/progression of the cancer, a delay to/prevention of onset of the cancer, a reduction in/delay to/prevention of tumor growth, a reduction in/delay to/prevention of tissue invasion, a reduction in/delay to/prevention of metastasis, a reduction in the severity of one or more symptoms of the cancer, a reduction in the number of cancer cells, a reduction in the cancer burden, a reduction in tumor size/volume, and/or an increase in survival of subjects having the cancer (e.g. progression free survival or overall survival).

In accordance with various aspects of the present disclosure, a method of treating and/or preventing a cancer according to the present disclosure may comprise inhibiting the growth of a tumor, reducing the size/volume of a tumor and/or increasing the survival of a subject having the cancer.

In accordance with various aspects of the present disclosure, methods are provided which are for, or which comprise (e.g. in the context of treatment/prevention of a cancer, e.g. a cancer described herein), one or more of the following:
binding to cells expressing TROP2;
inhibiting the proliferation of TROP2-expressing cells;
killing cells expressing TROP2;
inhibiting tumor growth and/or reducing tumor size/volume, e.g. of a TROP2-expressing cancer; and/or
increasing the survival of subjects having a cancer, e.g. a TROP2-expressing cancer.

Also provided are antigen-binding molecules and compositions according to the present disclosure for use in such methods, and the use of antigen-binding molecules and compositions according to the present disclosure in manufacture of compositions (e.g. medicaments) for use in such methods. It will be appreciated that the methods typically comprise administering an antigen-binding molecule according to the present disclosure to a subject.

Similarly, one or more of the following may be observed in a subject following therapeutic or prophylactic intervention in accordance with the present disclosure (e.g. compared to the level/number/proportion etc. prior to intervention):
inhibition of proliferation of TROP2-expressing cells;
killing of cells expressing TROP2;
inhibition of tumor growth and/or reduction of tumor size/volume, e.g. of a TROP2-expressing cancer; and/or
increased survival of a subject having a cancer, e.g. a TROP2-expressing cancer.

In some embodiments, therapeutic/prophylactic intervention in accordance with the present disclosure may be described as being ‘associated with’ one or more of the effects described in the preceding paragraph. The skilled person is readily able to evaluate such properties using techniques that are routinely practiced in the art.

Administration of the antigen-binding molecules and compositions of the present disclosure is preferably in a ‘therapeutically-effective’ or ‘prophylactically-effective’ amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (Ed. A. Adejare), 23^{rd} Edition (2020), Academic Press.

Administration of the antigen-binding molecules and compositions of the present disclosure may be e.g. parenteral, systemic, topical, intracavitary, intravascular, intravenous, intra-arterial, intramuscular, intrathecal, intraocular, intraconjunctival, intratumoral, subcutaneous, intradermal, oral or transdermal. Administration may be by injection, infusion or ingestion.

In some aspects and embodiments, articles of the present disclosure may be administered to a tissue/organ of interest (e.g. a tissue/organ affected by the disease/condition, e.g. a tissue/organ in which symptoms of the disease/condition manifest). In some aspects and embodiments, articles of the present disclosure may be administered to the blood (i.e. intravenous/intra-arterial administration) by injection or infusion (e.g. via cannula), or may be administered subcutaneously or orally. In some aspects and embodiments, articles of the present disclosure may be administered to a tumor.

In some embodiments, therapeutic or prophylactic intervention according to the present disclosure may further comprise administering another agent for the treatment/prevention of the relevant disease/condition. Administration of antigen-binding molecules and compositions described herein may be alone or in combination with other treatments, either simultaneously or sequentially, dependent upon the condition to be treated. Simultaneous administration refers to administration with another therapeutic agent together, for example as a pharmaceutical composition containing both agents (combined preparation), or immediately after each other (e.g. within 1, 4, 6, 8 or 12 hours) and optionally via the same route of administration (e.g. to the same tissue, artery, vein or other blood vessel). Sequential administration refers to administration of one agent followed after a given time interval by separate administration of another agent. It is not required that the two agents are administered by the same route, although this is the case in some embodiments. The time interval may be any time interval.

Multiple doses of the antigen-binding molecules and compositions may be provided. Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

### Methods of Detection

The present disclosure also provides the articles of the present disclosure for use in methods for detecting, localising or imaging TROP2, or cells expressing TROP2.

The antigen-binding molecules described herein may be used in methods that involve detecting binding of the antigen-binding molecule to TROP2. Such methods may involve detection of the bound complex of the antigen-binding molecule and TROP2. It will be appreciated that the TROP2 may be TROP2 expressed by a cell, e.g. in or at the cell surface of a cell expressing TROP2.

As such, a method is provided, comprising contacting a sample containing, or suspected to contain, TROP2, and detecting the formation of a complex of the antigen-binding molecule and TROP2. Also provided is a method comprising contacting a sample containing, or suspected to contain, a cell expressing TROP2, and detecting the formation of a complex of the antigen-binding molecule and a cell expressing TROP2.

Suitable method formats are well known in the art, including immunoassays such as sandwich assays, e.g. ELISA. The methods may involve labelling the antigen-binding molecule, or target(s), or both, with a detectable moiety, e.g. a fluorescent label, phosphorescent label, luminescent label, immuno-detectable label, radiolabel, chemical, nucleic acid or enzymatic label as described herein. Detection techniques are well known to those of skill in the art and can be selected to correspond with the labelling agent.

Methods comprising detecting TROP2, or cells expressing TROP2, include methods for diagnosing/prognosing a disease/condition described herein.

Methods of this kind may be performed in vitro on a patient sample, or following processing of a patient sample. Once the sample is collected, the patient is not required to be present for the in vitro method to be performed, and therefore the method may be one which is not practised on the human or animal body. In some embodiments, the method is performed in vivo.

Such methods may involve detecting or quantifying TROP2 and/or cells expressing TROP2, e.g. in a patient sample. Where the method comprises quantifying the relevant factor, the method may further comprise comparing the determined amount against a standard or reference value as part of the diagnostic or prognostic evaluation. Other diagnostic/prognostic tests may be used in conjunction with those described herein to enhance the accuracy of the diagnosis or prognosis or to confirm a result obtained by using the tests described herein.

Detection in a sample may be used for the purpose of diagnosis of a disease/condition (e.g. a cancer), predisposition to a disease/condition, or for providing a prognosis (prognosticating) for a disease/condition, e.g. a disease/condition described herein. The diagnosis or prognosis may relate to an existing (previously diagnosed) disease/condition.

A sample may be taken from any tissue or bodily fluid. The sample may comprise or may be derived from: a quantity of blood; a quantity of serum derived from the individual's blood which may comprise the fluid portion of the blood obtained after removal of the fibrin clot and blood cells; a tissue sample or biopsy; pleural fluid; cerebrospinal fluid (CSF); or cells isolated from said individual. In some embodiments, the sample may be obtained or derived from a tissue or tissues which are affected by the disease/condition (e.g. tissue or tissues in which symptoms of the disease manifest, or which are involved in the pathogenesis of the disease/condition).

A subject may be selected for diagnostic/prognostic evaluation based on the presence of symptoms indicative of a disease/condition described herein, or based on the subject being considered to be at risk of developing a disease/condition described herein.

The present disclosure also provides methods for selecting/stratifying a subject for treatment with a TROP2-targeted agent. In some embodiments a subject is selected for treatment/prevention in accordance with the methods of the present disclosure, or is identified as a subject which would benefit from such treatment/prevention, based on detection/quantification of TROP2, or cells expressing TROP2, e.g. in a sample obtained from the individual.

### Subjects

The subject in accordance with aspects described herein may be any animal or human. The subject is preferably mammalian, more preferably human. The subject may be a non-human mammal, but is more preferably human. The subject may be male or female. The subject may be a patient. A subject may have been diagnosed with a disease or condition requiring treatment (e.g. a cancer, e.g. a cancer described herein), may be suspected of having such a disease/condition, or may be at risk of developing/contracting such a disease/condition.

In some embodiments, the subject to be treated according to a therapeutic or prophylactic method of the present disclosure herein is a subject having, or at risk of developing, a cancer, e.g. a cancer described herein. In embodiments according to the present disclosure, a subject may be selected for treatment according to the methods based on characterisation for certain markers of such disease/condition.

In some embodiments, a patient may be selected for treatment described herein based on the detection of a cancer expressing/overexpressing TROP2, e.g. in a sample obtained from the subject (e.g. a biopsy, e.g. of a tumor).

In some embodiments, a patient may be selected for treatment described herein based on detection of a relapsed/refractory cancer subsequent to administration of a therapy for the cancer, e.g. a first line therapy for the cancer or a second line therapy for the cancer. In some embodiments, a patient may be selected for treatment described herein based on detection of a relapsed/refractory cancer subsequent to administration of the standard-of-care therapy (e.g. the first line standard of care) for the given cancer. In some embodiments, a patient may be selected for treatment described herein based on detection of a relapsed/refractory cancer subsequent to administration of a therapy for the cancer comprising a TROP2 antibody drug conjugate (e.g. sacituzumab govitecan).

### Kits

The present disclosure also provides kits of parts. A kit according to the present disclosure may comprise components for performing a method described herein, in whole or in part.

The kit may have at least one container having a predetermined quantity of an antigen-binding molecule or composition described herein.

In some aspects of the present disclosure a kit of parts is provided. In some embodiments, the kit may comprise an antigen-binding molecule or composition described herein, and which may be provided in a predetermined quantity.

The kit may provide an antigen-binding molecule or composition described herein together with instructions for administration to a patient in order to treat a specified disease/condition (e.g. a disease/condition described herein, e.g. a cancer).

The kit may provide an antigen-binding moiety according to the disclosure, and a linker-payload moiety according to the present disclosure. The kit may further comprise reagents for conjugating the antigen-binding moiety and the linker-payload moiety.

The kit may further comprise reagents, buffers and/or standards required for execution of a method according to the present disclosure. Kits according to the present disclosure may include instructions for use, e.g. in the form of an instruction booklet or leaflet. The instructions may include a protocol for performing any one or more of the methods described herein.

### Sequence Identity

As used herein, ‘sequence identity’ refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6 (298)) and MAFFT (Katoh and Standley 2013, Molecular Biology and Evolution, 30 (4) 772-780) software. When using such software, the default parameters, e.g. for gap penalty and extension penalty, are preferably used.

Sequences

| SEQ ID | | |
|---|---|---|
| NO: | DESCRIPTION | SEQUENCE |
| 1 | Human TROP2 | MARGPGLAPPPLRLPLLLLVLAAVTGHTAAQDNCTCPTNKMTVCSPDGPGGRCQCRALGSGMAVDCSTLT |
| | (P09758 v3 | SKCLLLKARMSAPKNARTLVRPSEHALVDNDGLYDPDCDPEGRFKARQCNQTSVCWCVNSVGVRRTDKG |
| | 2007 Nov. 13) | DLSLRCDELVRTHHILIDLRHRPTAGAFNHSDLDAELRRLFRERYRLHPKFVAAVHYEQPTIQIELRQNTSQK |
| | | AAGDVDIGDAAYYFERDIKGESLFQGRGGLDLRVRGEPLQVERTLIYYLDEIPPKFSMKRLTAGLIAVIVVVVV |
| | | ALVAGMAVLVITNRRKSGKYKKVEIKELGELRKEPSL |
| 2 | Mature human | HTAAQDNCTCPTNKMTVCSPDGPGGRCQCRALGSGMAVDCSTLTSKCLLLKARMSAPKNARTLVRPSEHA |
| | TROP2 | LVDNDGLYDPDCDPEGRFKARQCNQTSVCWCVNSVGVRRTDKGDLSLRCDELVRTHHILIDLRHRPTAGAF |
| | | NHSDLDAELRRLFRERYRLHPKFVAAVHYEQPTIQIELRQNTSQKAAGDVDIGDAAYYFERDIKGESLFQGR |
| | | GGLDLRVRGEPLQVERTLIYYLDEIPPKFSMKRLTAGLIAVIVVVVVALVAGMAVLVITNRRKSGKYKKVEIKEL |
| | | GELRKEPSL |
| 3 | Human TROP2 | MARGPGLAPPPLRLPLLLLVLAAVTG |
| | signal peptide | |
| 4 | Human TROP2 | HTAAQDNCTCPTNKMTVCSPDGPGGRCQCRALGSGMAVDCSTLTSKCLLLKARMSAPKNARTLVRPSEHA |
| | extracellular | LVDNDGLYDPDCDPEGRFKARQCNQTSVCWCVNSVGVRRTDKGDLSLRCDELVRTHHILIDLRHRPTAGAF |
| | domain | NHSDLDAELRRLFRERYRLHPKFVAAVHYEQPTIQIELRQNTSQKAAGDVDIGDAAYYFERDIKGESLFQGR |
| | | GGLDLRVRGEPLQVERTLIYYLDEIPPKFSMKRLT |
| 5 | Human TROP2 | HTAAQDNCTCPTNKMTVCSPDGPGGRCQCRALGSGMAVDCSTLTSKC |
| | cysteine-rich | |
| | domain (CRD) | |
| 6 | Human TROP2 | LLLKARMSAPKNARTLVRPSEHALVDNDGLYDPDCDPEGRFKARQCNQTSVCWCVNSVGVRRTDKGDLSL |
| | thyroglobulin | RCD |
| | type-1 domain | |
| | (TY-1) | |
| 7 | Human TROP2 | ELVRTHHILIDLRHRPTAGAFNHSDLDAELRRLFRERYRLHPKFVAAVHYEQPTIQIELRQNTSQKAAGDVDI |
| | cysteine-poor | GDAAYYFERDIKGESLFQGRGGLDLRVRGEPLQVERTLIYYLDEIPPKFSMKRLT |
| | domain (CPD) | |
| 8 | Human TROP2 | AGLIAVIVVVVVALVAGMAVLVI |
| | transmembrane | |
| | domain | |
| 9 | Human TROP2 | TNRRKSGKYKKVEIKELGELRKEPSL |
| | cytoplasmic | |
| | domain | |
| 10 | Sacituzumab HC | QVQLQQSGSELKKPGASVKVSCKASGYTFTNYGMNWVKQAPGQGLKWMGWINTYTGEPTYTDDFKGRFA |
| | | FSLDTSVSTAYLQISSLKADDTAVYFCARGGFGSSYWYFDVWGQGSLVTVSSASTKGPSVFPLAPSSKSTS |
| | | GGTAALGCLVKDYFPEPVTVSWNSGALTSGVHTFPAVLQSSGLYSLSSVVTVPSSSLGTQTYICNVNHKPS |
| | | NTKVDKRVEPKSCDKTHTCPPCPAPELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLYSKLTVDKSRWQQG |
| | | NVFSCSVMHEALHNHYTQKSLSLSPG |
| 11 | Sacituzumab κ | DIQLTQSPSSLSASVGDRVSITCKASQDVSIAVAWYQQKPGKAPKLLIYSASYRYTGVPDRFSGSGSGTDFT |
| | LC | LTISSLQPEDFAVYYCQQHYITPLTFGAGTKVEIKRTVAAPSVFIFPPSDEQLKSGTASVVCLLNNFYPREAKV |
| | | QWKVDNALQSGNSQESVTEQDSKDSTYSLSSTLTLSKADYEKHKVYACEVTHQGLSSPVTKSFNRGEC |
| 12 | Sacituzumab VH | QVQLQQSGSELKKPGASVKVSCKASGYTFTNYGMNWVKQAPGQGLKWMGWINTYTGEPTYTDDFKGRFA |
| | | FSLDTSVSTAYLQISSLKADDTAVYFCARGGFGSSYWYFDVWGQGSLVTVSS |
| 13 | Sacituzumab | GYTFTNYG |
| | HC-CDR1 | |
| 14 | Sacituzumab | INTYTGEP |
| | HC-CDR2 | |
| 15 | Sacituzumab | ARGGFGSSYWYFDV |
| | HC-CDR3 | |
| 16 | Sacituzumab | QVQLQQSGSELKKPGASVKVSCKAS |
| | HC-FR1 | |
| 17 | Sacituzumab | MNWVKQAPGQGLKWMGW |
| | HC-FR2 | |
| 18 | Sacituzumab | TYTDDFKGRFAFSLDTSVSTAYLQISSLKADDTAVYFC |
| | HC-FR3 | |
| 19 | Sacituzumab | WGQGSLVTVSS |
| | HC-FR4 | |
| 20 | Sacituzumab VL | DIQLTQSPSSLSASVGDRVSITCKASQDVSIAVAWYQQKPGKAPKLLIYSASYRYTGVPDRFSGSGSGTDFT |
| | | LTISSLQPEDFAVYYCQQHYITPLTFGAGTKVEIKRTV |
| 21 | Sacituzumab | QDVSIA |
| | LC-CDR1 | |
| 22 | Sacituzumab | SAS |
| | LC-CDR2 | |
| 23 | Sacituzumab | QQHYITPLT |
| | LC-CDR3 | |
| 24 | Sacituzumab | DIQLTQSPSSLSASVGDRVSITCKAS |
| | LC-FR1 | |
| 25 | Sacituzumab | VAWYQQKPGKAPKLLIY |
| | LC-FR2 | |
| 26 | Sacituzumab | YRYTGVPDRFSGSGSGTDFTLTISSLQPEDFAVYYC |
| | LC-FR3 | |
| 27 | Sacituzumab | FGAGTKVEIKRTV |
| | LC-FR4 | |
| 28 | Datopotamab | QVQLVQSGAEVKKPGASVKVSCKASGYTFTTAGMQWVRQAPGQGLEWMGWINTHSGVPKYAEDFKGRV |
| | HC | TISADTSTSTAYLQLSSLKSEDTAVYYCARSGFGSSYWYFDVWGQGTLVTVSSASTKGPSVFPLAPSSKSTS |
| | | GGTAALGCLVKDYFPEPVTVSWNSGALTSGVHTFPAVLQSSGLYSLSSVVTVPSSSLGTQTYICNVNHKPS |
| | | NTKVDKRVEPKSCDKTHTCPPCPAPELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCKVSNKALPAPIEKTISKAKGQPREPQVY |
| | | TLPPSREEMTKNQVSLTCLVKGFYPSDIAVEWESNGQPENNYKTTPPVLDSDGSFFLYSKLTVDKSRWQQG |
| | | NVFSCSVMHEALHNHYTQKSLSLSPG |
| 29 | Datopotamab κ | DIQMTQSPSSLSASVGDRVTITCKASQDVSTAVAWYQQKPGKAPKLLIYSASYRYTGVPSRFSGSGSGTDF |
| | LC | TLTISSLQPEDFAVYYCQQHYITPLTFGQGTKLEIKRTVAAPSVFIFPPSDEQLKSGTASVVCLLNNFYPREAK |
| | | VQWKVDNALQSGNSQESVTEQDSKDSTYSLSSTLTLSKADYEKHKVYACEVTHQGLSSPVTKSFNRGEC |
| 30 | Datopotamab | QVQLVQSGAEVKKPGASVKVSCKASGYTFTTAGMQWVRQAPGQGLEWMGWINTHSGVPKYAEDFKGRV |
| | VH | TISADTSTSTAYLQLSSLKSEDTAVYYCARSGFGSSYWYFDVWGQGTLVTVSS |
| 31 | Datopotamab | GYTFTTAG |
| | HC-CDR1 | |
| 32 | Datopotamab | INTHSGVP |
| | HC-CDR2 | |
| 33 | Datopotamab | ARSGFGSSYWYFDV |
| | HC-CDR3 | |
| 34 | Datopotamab | QVQLVQSGAEVKKPGASVKVSCKAS |
| | HC-FR1 | |
| 35 | Datopotamab | MQWVRQAPGQGLEWMGW |
| | HC-FR2 | |
| 36 | Datopotamab | KYAEDFKGRVTISADTSTSTAYLQLSSLKSEDTAVYYC |
| | HC-FR3 | |
| 37 | Datopotamab | WGQGTLVTVSS |
| | HC-FR4 | |
| 38 | Datopotamab VL | DIQMTQSPSSLSASVGDRVTITCKASQDVSTAVAWYQQKPGKAPKLLIYSASYRYTGVPSRFSGSGSGTDF |
| | | TLTISSLQPEDFAVYYCQQHYITPLTFGQGTKLEIKRTV |
| 39 | Datopotamab | QDVSTA |
| | LC-CDR1 | |
| 40 | Datopotamab | SAS |
| | LC-CDR2 | |
| 41 | Datopotamab | QQHYITPLT |
| | LC-CDR3 | |
| 42 | Datopotamab | DIQMTQSPSSLSASVGDRVTITCKAS |
| | LC-FR1 | |
| 43 | Datopotamab | VAWYQQKPGKAPKLLIY |
| | LC-FR2 | |
| 44 | Datopotamab | YRYTGVPSRFSGSGSGTDFTLTISSLQPEDFAVYYC |
| | LC-FR3 | |
| 45 | Datopotamab | FGQGTKLEIKRTV |
| | LC-FR4 | |
| 46 | Human IgG1 | ASTKGPSVFPLAPSSKSTSGGTAALGCLVKDYFPEPVTVSWNSGALTSGVHTFPAVLQSSGLYSLSSVVTVP |
| | constant region | SSSLGTQTYICNVNHKPSNTKVDKKVEPKSCDKTHTCPPCPAPELLGGPSVFLFPPKPKDTLMISRTPEVTC |
| | (IGHG1; | VVVDVSHEDPEVKFNWYVDGVEVHNAKTKPREEQYNSTYRVVSVLTVLHQDWLNGKEYKCKVSNKALPAP |
| | UniProt:P01857- | IEKTISKAKGQPREPQVYTLPPSRDELTKNQVSLTCLVKGFYPSDIAVEWESNGQPENNYKTTPPVLDSDGS |
| | 1, v1) | FFLYSKLTVDKSRWQQGNVFSCSVMHEALHNHYTQKSLSLSPGK |
| 47 | CH1 IgG1 | ASTKGPSVFPLAPSSKSTSGGTAALGCLVKDYFPEPVTVSWNSGALTSGVHTFPAVLQSSGLYSLSSVVTVP |
| | (positions 1-98 | SSSLGTQTYICNVNHKPSNTKVDKKV |
| | of P01857-1, v1) | |
| 48 | Hinge IgG1 | EPKSCDKTHTCP |
| | (positions 99- | |
| | 110 of P01857- | |
| | 1, v1) | |
| 49 | CH2 IgG1 | PCPAPELLGGPSVFLEPPKPKDTLMISRTPEVTCVVVDVSHEDPEVKFNWYVDGVEVHNAKTKPREEQYNS |
| | (positions 111- | TYRVVSVLTVLHQDWLNGKEYKCKVSNKALPAPIEKTISKAK |
| | 223 of P01857- | |
| | 1, v1) | |
| 50 | CH3 IgG1 | GQPREPQVYTLPPSRDELTKNQVSLTCLVKGFYPSDIAVEWESNGQPENNYKTTPPVLDSDGSFFLYSKLT |
| | (positions 224- | VDKSRWQQGNVFSCSVMHEALHNHYTQKSLSLSPGK |
| | 330 of P01857- | |
| | 1, v1) | |
| 51 | Human IgG1 | ASTKGPSVFPLAPSSKSTSGGTAALGCLVKDYFPEPVTVSWNSGALTSGVHTFPAVLQSSGLYSLSSVVTVP |
| | constant region | SSSLGTQTYICNVNHKPSNTKVDKRVEPKSCDKTHTCPPCPAPELLGGPSVFLFPPKPKDTLMISRTPEVTC |
| | G1m3 allotype | VVVDVSHEDPEVKFNWYVDGVEVHNAKTKPREEQYNSTYRVVSVLTVLHQDWLNGKEYKCKVSNKALPAP |
| | (K214R, D356E | IEKTISKAKGQPREPQVYTLPPSREEMTKNQVSLTCLVKGFYPSDIAVEWESNGQPENNYKTTPPVLDSDGS |
| | and L358M (EU | FFLYSKLTVDKSRWQQGNVFSCSVMHEALHNHYTQKSLSLSPGK |
| | numbering) | |
| | relative to | |
| | P01857-1) | |
| 52 | CH1 IgG1 G1m3 | ASTKGPSVFPLAPSSKSTSGGTAALGCLVKDYFPEPVTVSWNSGALTSGVHTFPAVLQSSGLYSLSSVVTVP |
| | allotype | SSSLGTQTYICNVNHKPSNTKVDKRV |
| 53 | CH3 IgG1 G1m3 | GQPREPQVYTLPPSREEMTKNQVSLTCLVKGFYPSDIAVEWESNGQPENNYKTTPPVLDSDGSFFLYSKLT |
| | allotype | VDKSRWQQGNVFSCSVMHEALHNHYTQKSLSLSPGK |
| 54 | Human IgG1 | PCPAPELLGGPSVFLFPPKPKDTLMISRTPEVTCVVVDVSHEDPEVKFNWYVDGVEVHNAKTKPREEQYNS |
| | CH2-CH3 region | TYRVVSVLTVLHQDWLNGKEYKCKVSNKALPAPIEKTISKAKGQPREPQVYTLPPSRDELTKNQVSLTCLVK |
| | | GFYPSDIAVEWESNGQPENNYKTTPPVLDSDGSFFLYSKLTVDKSRWQQGNVFSCSVMHEALHNHYTQKS |
| | | LSLSPGK |
| 55 | Human IgG1 | PCPAPELLGGPSVFLFPPKPKDTLMISRTPEVTCVVVDVSHEDPEVKFNWYVDGVEVHNAKTKPREEQYNS |
| | G1m3 allotype | TYRVVSVLTVLHQDWLNGKEYKCKVSNKALPAPIEKTISKAKGQPREPQVYTLPPSREEMTKNQVSLTCLVK |
| | CH2-CH3 region | GFYPSDIAVEWESNGQPENNYKTTPPVLDSDGSFFLYSKLTVDKSRWQQGNVFSCSVMHEALHNHYTQKS |
| | | LSLSPG |
| 56 | Cκ CL (IGKC; | RTVAAPSVFIFPPSDEQLKSGTASVVCLLNNFYPREAKVQWKVDNALQSGNSQESVTEQDSKDSTYSLSST |
| | UniProt: | LTLSKADYEKHKVYACEVTHQGLSSPVTKSFNRGEC |
| | P01834-1, v2)) | |
| 57 | Cλ CL1 (IGLC1; | GQPKANPTVTLFPPSSEELQANKATLVCLISDFYPGAVTVAWKADGSPVKAGVETTKPSKQSNNKYAASSYL |
| | UniProt: | SLTPEQWKSHRSYSCQVTHEGSTVEKTVAPTECS |
| | P0CG04-1, v1) | |
| 58 | Cλ CL2 (IGLC2; | GQPKAAPSVTLFPPSSEELQANKATLVCLISDFYPGAVTVAWKADSSPVKAGVETTTPSKQSNNKYAASSYL |
| | UniProt: | SLTPEQWKSHRSYSCQVTHEGSTVEKTVAPTECS |
| | P0DOY2-1, v1) | |
| 59 | Cλ CL3 (IGLC3; | GQPKAAPSVTLFPPSSEELQANKATLVCLISDFYPGAVTVAWKADSSPVKAGVETTTPSKQSNNKYAASSYL |
| | UniProt: | SLTPEQWKSHKSYSCQVTHEGSTVEKTVAPTECS |
| | P0DOY3-1, v1) | |
| 60 | Cλ CL6 (IGLC6; | GQPKAAPSVTLFPPSSEELQANKATLVCLISDFYPGAVKVAWKADGSPVNTGVETTTPSKQSNNKYAASSYL |
| | UniProt: | SLTPEQWKSHRSYSCQVTHEGSTVEKTVAPAECS |
| | P0CF74-1, v1) | |
| 61 | Cλ CL7 (IGLC7; | GQPKAAPSVTLFPPSSEELQANKATLVCLVSDFNPGAVTVAWKADGSPVKVGVETTKPSKQSNNKYAASSY |
| | UniProt: | LSLTPEQWKSHRSYSCRVTHEGSTVEKTVAPAECS |
| | A0M8Q6-1, v3) | |
| 62 | Human IgG1 | ASTKGPSVFPLAPSSKSTSGGTAALGCLVKDYFPEPVTVSWNSGALTSGVHTFPAVLQSSGLYSLSSVVTVP |
| | constant region | SSSLGTQTYICNVNHKPSNTKVDKKVEPKSCDKTHTCPPCPAPELLGGPSVFLFPPKPKDTLMISRTPEVTC |
| | (IGHG1; | VVVDVSHEDPEVKFNWYVDGVEVHNAKTKPREEQYASTYRVVSVLTVLHQDWLNGKEYKCKVSNKALPAPI |
| | UniProt:P01857- | EKTISKAKGQPREPQVYTLPPSRDELTKNQVSLTCLVKGFYPSDIAVEWESNGQPENNYKTTPPVLDSDGSF |
| | 1, v1) | FLYSKLTVDKSRWQQGNVFSCSVMHEALHNHYTQKSLSLSPGK |
| | comprising | |
| | N297A | |
| 63 | Human IgG1 | ASTKGPSVFPLAPSSKSTSGGTAALGCLVKDYFPEPVTVSWNSGALTSGVHTFPAVLQSSGLYSLSSVVTVP |
| | constant region | SSSLGTQTYICNVNHKPSNTKVDKRVEPKSCDKTHTCPPCPAPELLGGPSVFLFPPKPKDTLMISRTPEVTC |
| | G1m3 allotype | VVVDVSHEDPEVKFNWYVDGVEVHNAKTKPREEQYASTYRVVSVLTVLHQDWLNGKEYKCKVSNKALPAPI |
| | (K214R, D356E | EKTISKAKGQPREPQVYTLPPSREEMTKNQVSLTCLVKGFYPSDIAVEWESNGQPENNYKTTPPVLDSDGS |
| | and L358M (EU | FFLYSKLTVDKSRWQQGNVFSCSVMHEALHNHYTQKSLSLSPGK |
| | numbering) | |
| | relative to | |
| | P01857-1) | |
| | comprising | |
| | N297A | |
| 64 | CH2 IgG1 | PCPAPELLGGPSVFLFPPKPKDTLMISRTPEVTCVVVDVSHEDPEVKFNWYVDGVEVHNAKTKPREEQYAS |
| | comprising | TYRVVSVLTVLHQDWLNGKEYKCKVSNKALPAPIEKTISKAK |
| | N297A | |
| 65 | Human IgG1 | PCPAPELLGGPSVFLFPPKPKDTLMISRTPEVTCVVVDVSHEDPEVKFNWYVDGVEVHNAKTKPREEQYAS |
| | CH2-CH3 region | TYRVVSVLTVLHQDWLNGKEYKCKVSNKALPAPIEKTISKAKGQPREPQVYTLPPSRDELTKNQVSLTCLVK |
| | comprising | GFYPSDIAVEWESNGQPENNYKTTPPVLDSDGSFFLYSKLTVDKSRWQQGNVFSCSVMHEALHNHYTQKS |
| | N297A | LSLSPGK |
| 66 | Human IgG1 | PCPAPELLGGPSVFLFPPKPKDTLMISRTPEVTCVVVDVSHEDPEVKFNWYVDGVEVHNAKTKPREEQYAS |
| | G1m3 allotype | TYRVVSVLTVLHQDWLNGKEYKCKVSNKALPAPIEKTISKAKGQPREPQVYTLPPSREEMTKNQVSLTCLVK |
| | CH2-CH3 region | GFYPSDIAVEWESNGQPENNYKTTPPVLDSDGSFFLYSKLTVDKSRWQQGNVFSCSVMHEALHNHYTQKS |
| | comprising | LSLSPG |
| | N297A | |
| 67 | Sacituzumab | QGRGGLDLRVRGEPLQ |
| | epitope | |

The present disclosure includes the combination of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise,’ and variations such as ‘comprises’ and ‘comprising,’ will be understood to imply the inclusion of a stated integer or step or group of integers or steps but not the exclusion of any other integer or step or group of integers or steps.

It must be noted that, as used in the specification and the appended claims, the singular forms ‘a’, ‘an’, and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about’, it will be understood that the particular value forms another embodiment.

Where a nucleic acid sequence is disclosed or referred to herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

Values may be expressed herein as ‘about’ a particular value. Similarly, ranges may be expressed herein as from ‘about’ a particular value, and/or to ‘about’ another particular value. The term ‘about’ in relation to a numerical value is optional, and means for example +/−10%. By way of illustration, reference e.g. to ‘about 10%’ is to be construed as 9% to 11%. In instances herein where ‘about’ is recited, the value it precedes is also specifically contemplated. By way of illustration, reference e.g. to ‘about 10%’ also specifically contemplates 10%.

### BRIEF DESCRIPTION OF THE FIGURES

Embodiments and experiments illustrating the principles of the invention will now be discussed with reference to the accompanying figures.

FIG. 1A shows % inhibition for a TOP1 inhibitor and an ATR inhibitor alone and in combination in HCT-116 cells.

FIG. 1B shows % inhibition for a TOP1 inhibitor and a CHK1 inhibitor alone and in combination in HCT-116 cells.

FIG. 1C shows % inhibition for a TOP1 inhibitor and an ATR inhibitor alone and in combination in HEC-1B cells.

FIG. 1D shows % inhibition for a TOP1 inhibitor and a CHK1 inhibitor alone and in combination in HEC-1B cells.

FIG. 2A shows the Loewe synergy score for exatecan and berzosertib (upper panel) and the % inhibition for exatecan and berzosertib alone and in combination (lower panel) in HEC1B cells.

FIG. 2B shows the Loewe synergy score for exatecan and berzosertib (upper panel) and the % inhibition for exatecan and berzosertib alone and in combination (lower panel) in HCT-116 cells.

FIG. 3A shows the level of pATR, pCHK1, pH2AX and CHK1 in JIMT-1 cells untreated or treated with 100 nM exatecan, 100 nM berzosertib, or 100 nM exatecan and 100 nM berzosertib in combination.

FIG. 3B shows the level of pATR, pCHK1, pH2AX and CHK1 in HCT-116 cells untreated or treated with 75 nM exatecan, 75 nM berzosertib, or 75 nM exatecan and 75 nM berzosertib in combination.

FIG. 4A shows survival of Sprague-Dawley rats following administration of vehicle, exatecan (1, 3, 10 or 30 mg/kg), berzosertib (35 mg/kg) or exatecan and berzosertib in combination (1, 3, 10 or 30 mg/kg exatecan+35 mg/kg berzosertib).

FIG. 4B shows body weight change in Sprague-Dawley rats following administration of vehicle, exatecan (1, 3, 10 or 30 mg/kg), berzosertib (35 mg/kg) or exatecan and berzosertib in combination (1, 3, 10 or 30 mg/kg exatecan+35 mg/kg berzosertib).

FIG. 5A shows results of hematology assessments in Sprague-Dawley rats following administration of vehicle, exatecan (1, 3, 10 or 30 mg/kg), berzosertib (35 mg/kg) or exatecan and berzosertib in combination (1, 3, 10 or 30 mg/kg exatecan+35 mg/kg berzosertib).

FIG. 5B shows results of clinical chemistry assessments in Sprague-Dawley rats following administration of vehicle, exatecan (1, 3, 10 or 30 mg/kg), berzosertib (35 mg/kg) or exatecan and berzosertib in combination (1, 3, 10 or 30 mg/kg exatecan+35 mg/kg berzosertib).

### EXAMPLES

### Example 1—In Vitro Combination of DDR Inhibitors and TOP1 Inhibitors

In order to further demonstrate the benefits of combining DDR inhibitors and TOP1 inhibitors, certain inhibitors were combined in a two-dimensional proliferation assay as follows.

### Cell Lines

HCT-116
HEC-1B

### Test Compounds

Exatecan (MedChemExpress, #HY-13631)
Ceralasertib (MedChemExpress, #HY-19323)
Prexasertib (MedChemExpress, #HY-18174)

### Controls

Vehicle control (0.25% DMSO)
Only Cells

### Detecting Reagent

Cell Titre Glo 2.0, Promega #G9243

The cells were seeded in wells of a white 96-well plate at a density of 7,000 cells per well for HEC-1B cells, and 3,000 cells per well for HCT-116 cells, with 150 μl of media in each well. For the single compound experiment, 25 μl of the test compound and 25 μl of media were added to the respective wells at varying concentrations. For the synergy experiment, 25 μl of compound 1 and 25 μl of another compound were added to the respective wells at different concentrations. The plates were then incubated for 3 days at 37 °C with 5% CO₂. Following incubation, 50 μl of the detection reagent was added per well and shaken at 600 rpm for 20 minutes. The resulting luminescence was measured using Perkin Elmer Victor Nivo, and percent inhibition was calculated using the following equation:

% Inhibition=100−((Lum_{Treatment}/Lum_{Vehicle})*100)

Synergy between the test compounds (Loewe synergy score) was then determined using the Synergy Finder tool at synergyfinder.fimm.fi. A Loewe synergy score of above 10 indicates synergy, between 10 and −10 indicates an additive effect, and below −10 indicates antagonism.

### A: Exatecan and Ceralasertib (an ATR Inhibitor) in HCT-116 Cells

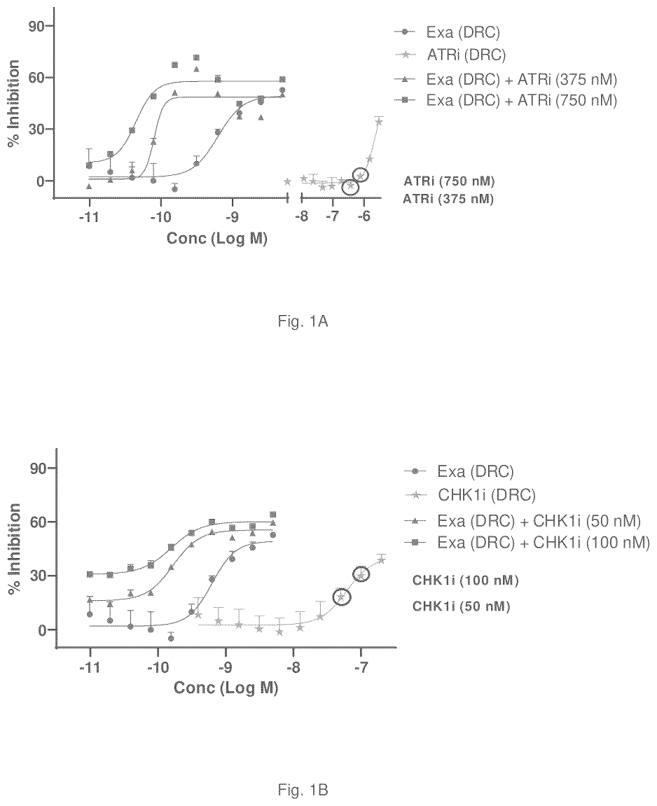

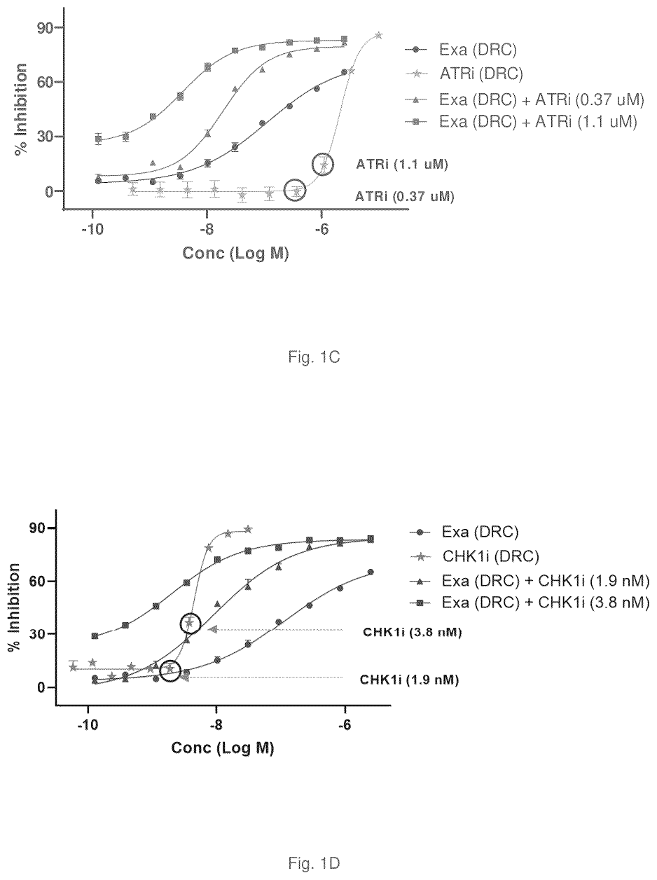

FIG. 1A shows the % inhibition for exatecan and ceralasertib alone and in combination. The Loewe synergy score is shown in table 1A below. The IC₅₀ for exatecan alone was 0.626 nM, when combined with ceralasertib at 375 nM was 0.080 nM and when combined with ceralasertib at 750 nM was 0.044 nM.

TABLE 1A

| | | | | | | | | | | | |
|---|---|---|---|---|---|---|---|---|---|---|---|
| Ceralasertib | 3000 | 14.47 | 7.75 | 14.24 | 22.99 | 29.46 | 28.14 | 19.70 | 14.41 | 18.67 | 26.21 |
| (nM) | 1500 | 16.96 | 26.24 | 1.73 | 14.76 | 26.42 | 25.94 | 16.68 | 6.20 | 9.53 | 20.48 |
| | 750 | 6.37 | 12.63 | 25.21 | 42.94 | 17.98 | 22.09 | 9.32 | 0.50 | −1.53 | 9.67 |
| | 375 | −5.16 | −1.60 | 4.08 | 19.51 | 1.90 | 15.52 | 1.24 | −5.98 | −10.88 | 0.65 |
| | 188 | −7.41 | −4.77 | −3.19 | −2.33 | 20.61 | 1.32 | 11.36 | −6.17 | −12.77 | −3.84 |
| | 94 | −8.44 | −6.34 | −3.72 | −6.87 | 3.28 | 20.33 | 9.72 | −4.78 | −10.01 | −5.03 |
| | 47 | −3.10 | −2.85 | −1.45 | −5.41 | −2.03 | 10.27 | 6.29 | −4.23 | −7.76 | −2.24 |
| | 23 | −3.04 | 0.99 | 3.88 | −0.49 | −0.78 | 5.97 | 4.24 | −4.06 | −6.20 | −0.96 |
| | 12 | 1.29 | 5.83 | 4.58 | 2.40 | 3.49 | 5.59 | 5.02 | −1.15 | −3.59 | 0.63 |
| | 6 | 5.29 | 5.46 | 6.95 | 4.14 | 9.33 | 10.49 | 9.10 | 1.97 | −1.21 | 4.21 |
| | | 0.009 | 0.019 | 0.039 | 0.078 | 10.156 | 0.313 | 0.63 | 1.25 | 2.51 | 5.0 |

Exatecan (nM)

### B: Exatecan and Prexasertib (a CHK1 Inhibitor) in HCT-116 Cells

FIG. 1B shows the % inhibition for exatecan and prexasertib alone and in combination. The Loewe synergy score is shown in table 1B below. The IC₅₀ for exatecan alone was 0.626 nM, when combined with prexasertib at 50 nM was 0.166 nM and when combined with prexasertib at 100 nM was 0.158 nM. The IC₅₀ for prexasertib alone was 61.14 nM.

TABLE 1B

| | | | | | | | | | | | |
|---|---|---|---|---|---|---|---|---|---|---|---|
| Prexasertib | 200 | 1.44 | 0.54 | 4.30 | 4.73 | 0.55 | 10.69 | 13.40 | 10.59 | 12.37 | 16.76 |
| (nM) | 100 | −0.70 | −0.97 | 1.99 | 3.22 | 11.68 | 4.53 | 10.68 | 7.28 | 8.16 | 14.77 |
| | 50 | −0.93 | −3.65 | 1.15 | −0.38 | 11.15 | 18.31 | 5.15 | 1.93 | 4.48 | 10.30 |
| | 25.0 | −4.31 | −5.15 | −3.38 | −4.93 | 6.01 | 14.06 | 1.73 | 4.34 | −0.95 | 6.29 |
| | 12.5 | −5.23 | −5.36 | 1.16 | −8.54 | 2.10 | 10.25 | 18.44 | 3.76 | −3.49 | 3.33 |
| | 6.3 | −4.45 | −5.70 | −0.48 | −6.58 | −4.00 | 0.64 | 10.86 | 2.45 | −6.72 | −0.59 |
| | 3.13 | −4.52 | −3.53 | −2.03 | −5.26 | −4.81 | −5.45 | 5.90 | 2.56 | −6.85 | −2.46 |
| | 1.56 | −3.41 | −2.52 | 0.97 | −3.23 | −2.10 | 1.45 | 4.34 | 1.67 | −6.14 | −1.63 |
| | 0.78 | 0.04 | 1.41 | 5.32 | −2.11 | −0.21 | 2.92 | 5.53 | 0.01 | −4.94 | −2.74 |
| | 0.39 | −0.86 | 2.10 | 4.13 | 0.29 | 5.42 | 6.59 | 6.01 | −2.13 | −1.71 | 2.17 |
| | | 0.009 | 0.019 | 0.039 | 0.078 | 0.156 | \|0.313 | 0.63 | 1.25 | 2.51 | 5.0 |

Exatecan (nM)

### C: Exatecan and Ceralasertib (an ATR Inhibitor) in HEC-1B Cells

FIG. 1C shows the % inhibition for exatecan and ceralasertib alone and in combination. The Loewe synergy score is shown in table 1C below. The IC₅₀ for exatecan alone was 113.9 nM, when combined with ceralasertib at 0.37 UM was 19.01 nM and when combined with ceralasertib at 1.1 μM was 3.719 nM. The IC₅₀ for ceralasertib alone was 2.118 μM.

TABLE 1C

| | | | | | | | | | | | |
|---|---|---|---|---|---|---|---|---|---|---|---|
| Ceralasertib | 10000 | −3.21 | −4.57 | −0.59 | −0.59 | −2.38 | 0.19 | −1.02 | −2.42 | −6.30 | −2.55 |
| (nM) | 3333 | −0.77 | 1.10 | 9.54 | 12.30 | 14.87 | 17.21 | 16.73 | 14.82 | 11.81 | 11.81 |
| | 1111 | 13.74 | 13.98 | 23.52 | 30.96 | 41.65 | 42.89 | 35.75 | 28.82 | 21.90 | 16.60 |
| | 370 | 3.36 | 2.76 | 7.85 | 0.83 | 13.33 | 29.05 | 29.00 | 26.53 | 20.11 | 16.47 |
| | 123 | 0.07 | −0.28 | 2.87 | −4.88 | −1.40 | 8.40 | 12.10 | 11.47 | 10.53 | 8.84 |
| | 41 | 1.41 | −1.48 | 3.94 | −2.08 | 0.04 | 0.57 | 1.91 | 1.86 | 2.09 | 1.87 |
| | 13.7 | −0.91 | 2.38 | 6.41 | 1.37 | 3.06 | 1.41 | 0.37 | −0.97 | 0.36 | −0.21 |
| | 4.6 | 2.34 | 1.73 | 6.53 | 2.58 | 5.53 | 4.57 | 1.48 | 3.16 | 1.14 | 0.56 |
| | 1.5 | 6.29 | 4.36 | 7.14 | 7.64 | 6.22 | 5.52 | 2.93 | 3.25 | 2.36 | 0.23 |
| | 0.5 | 4.68 | 6.42 | 8.44 | 7.46 | 3.80 | 5.70 | 5.00 | 6.34 | 3.45 | 0.76 |
| | | 0.12 | 0.38 | 1.1 | 3.4 | 10 | 30 | 92 | 278 | 833 | 2500 |

Exatecan (nM)

### D: Exatecan and Prexasertib (a CHK1 Inhibitor) in HEC-1B Cells

FIG. 1D shows the % inhibition for exatecan and prexasertib alone and in combination. The Loewe synergy score is shown in table 1D below. The IC₅₀ for exatecan alone was 113.9 nM, when combined with prexasertib at 1.9 nM was 8.988 nM and when combined with prexasertib at 3.8 nM was 2.013 nM. The IC₅₀ for prexasertib alone was 4.466 nM.

TABLE 1D

| | | | | | | | | | | | |
|---|---|---|---|---|---|---|---|---|---|---|---|
| Prexasertib | 30 | −6.77 | −4.80 | −3.82 | −3.12 | −3.26 | −3.16 | −1.74 | −1.53 | −2.55 | −2.76 |
| (nM) | 15 | −6.37 | −6.95 | −4.81 | −5.04 | −4.40 | −3.88 | −2.38 | −1.03 | −2.55 | −1.84 |
| | 7.5 | −6.19 | −4.10 | −0.89 | 0.98 | 3.68 | 4.20 | 4.84 | 7.43 | 6.02 | 5.36 |
| | 3.8 | −7.79 | −1.21 | 8.67 | 20.88 | 31.37 | 30.83 | 26.49 | 23.84 | 18.35 | 15.01 |
| | 1.9 | −8.06 | −7.30 | −1.57 | 9.34 | 24.03 | 24.85 | 26.19 | 27.04 | 21.05 | 16.08 |
| | 0.9 | −7.29 | −6.70 | 1.87 | 1.71 | 9.99 | 8.60 | 15.58 | 18.82 | 18.26 | 14.51 |
| | 0.47 | −4.93 | −6.53 | 0.96 | 0.09 | 4.58 | 2.46 | 5.75 | 12.52 | 11.43 | 10.24 |
| | 0.23 | −2.37 | −4.55 | 3.79 | 1.13 | 5.15 | 1.37 | 3.30 | 6.28 | 6.12 | 5.68 |
| | 0.12 | −2.99 | −3.35 | 1.01 | 4.08 | 4.30 | 2.92 | 0.52 | 5.06 | 4.29 | 3.57 |
| | 0.06 | −0.84 | −2.67 | 4.13 | 6.75 | 6.19 | 3.18 | −0.08 | 4.14 | 1.20 | 0.78 |
| | | 0.12 | 0.38 | 1.1 | 3.4 | 10 | 30 | 92 | 278 | 833 | 2500 |

Exatecan (nM)

In further experiments an additional DDR inhibitor, Berzosertib (an ATR inhibitor), was combined with TOP1 inhibitor Exatecan, in a two-dimensional proliferation assay as follows.

### Cell Lines

HEC-1B (HTB-113)
HCT-116 (CCL-247)

### Test Compounds

Exatecan (MedChemExpress, #HY-13631)
Berzosertib (Selleckchem, #S7102)

### Detection

CellTiter-Glo® 2.0D Cell Viability Assay (Promega, #G9243)

The cells were seeded in 96-well white opaque plates in 150 μl of media, and incubated at 37 °C with 5% CO₂ for 24 hours. HEC-1B was seeded at 5000 cells/well and HCT-116 was seeded at 3000 cells/well The next day, 25 μl of exatecan, berzosertib, or exatecan and berzosertib were added to cells at varying concentrations and incubated for 3 days at 37 °C with 5% CO₂. Following incubation, 50 μl of CellTiter-Glo reagent was added to the plates and incubated for 25 mins with gentle shaking at 600 rpm. Cell viability was measured via Luminescence using Victor Nivo, PerkinElmer.

Background was subtracted using Luminescence values from wells having only media (without any cells). Percent inhibition was calculated using the formula 100−{(Lum of cells treated with drug/Lum of cells treated with buffer control)*100}(Lum=Luminescence). The LOEWE synergy score was calculated using the Synergy Finder tool at synergyfinder.fimm.fi.

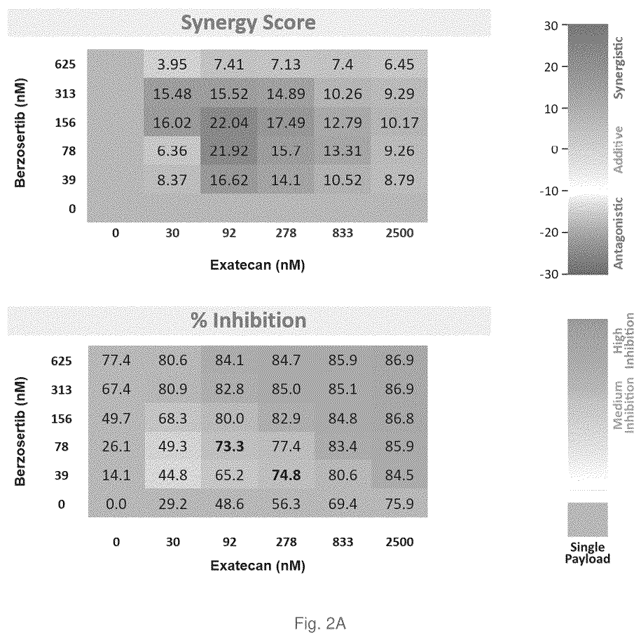

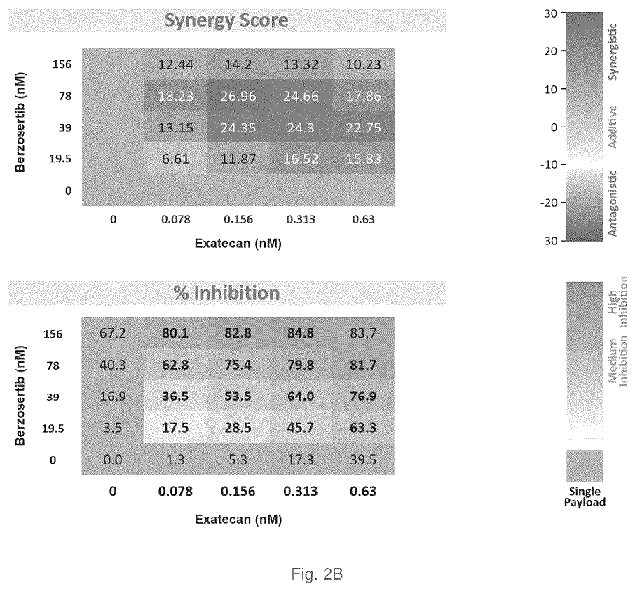

The results are shown in FIG. 2A and FIG. 2B. A synergistic effect is observed across a range of exatecan and berzosertib concentrations.

To summarise, exatecan was tested with two clinical stage ATR inhibitors (berzosertib and ceralasertib) for in vitro synergy in TOP1 inhibitor-resistant cell line HEC1B and TOP1 inhibitor-sensitive cell line HCT116. Both berzosertib and ceralasertib show synergy with exatecan across a wide range of concentrations tested in the cell lines. The effects in HEC1B demonstrate the TOP1 inhibitor and DDR inhibitor combination can sensitize inherently resistant cells to TOP1 inhibitor therapy.

### Example 2—Synthesis of Linker-Payload Molecule-1 (LP-1)

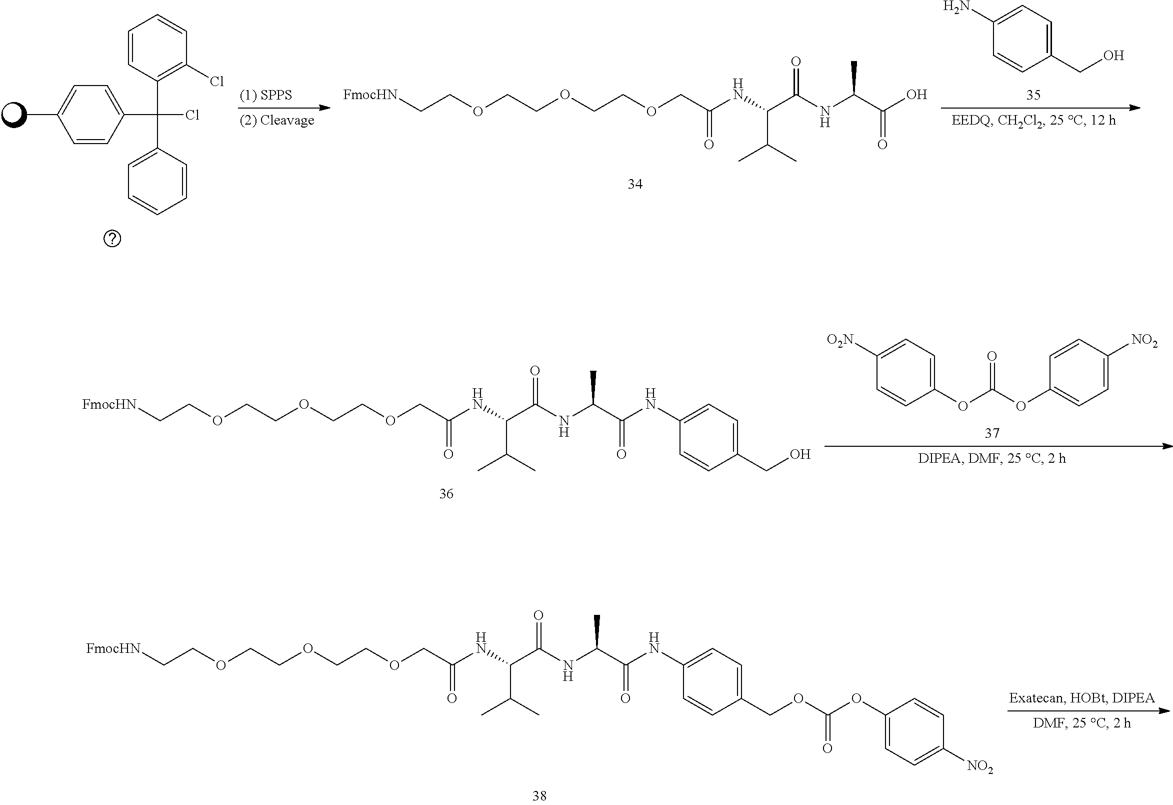

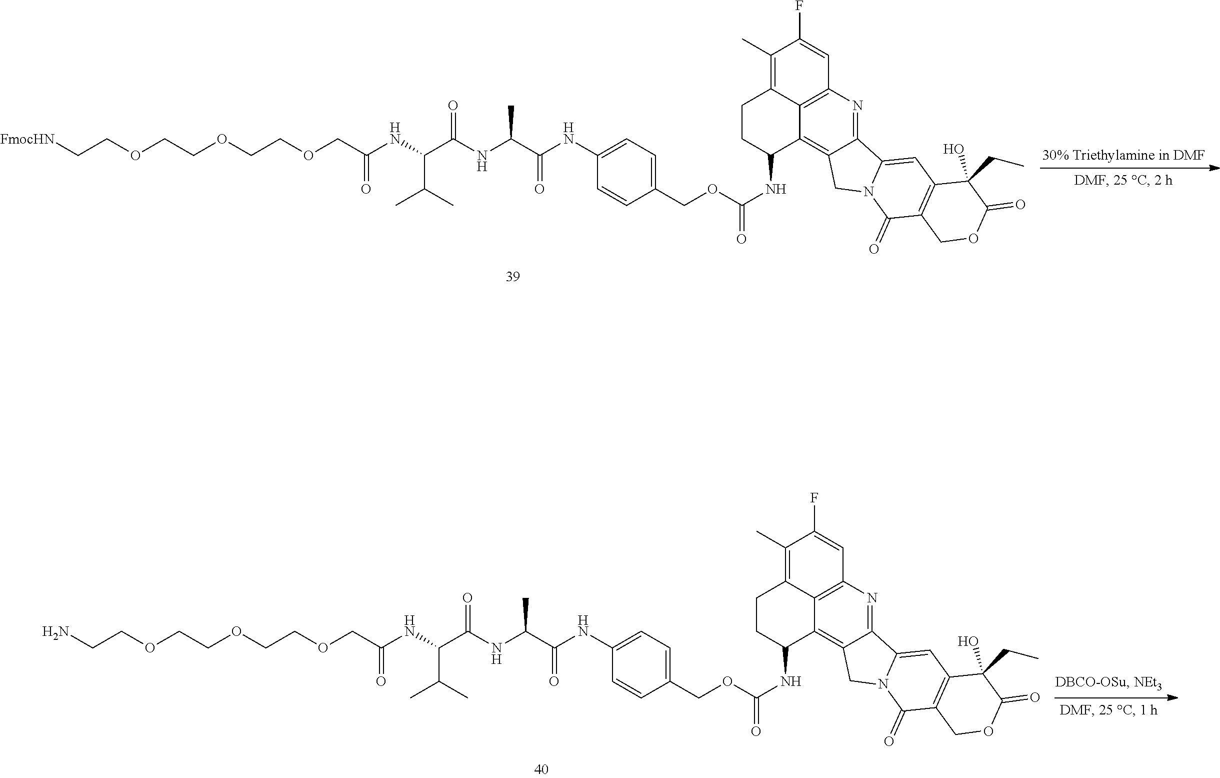

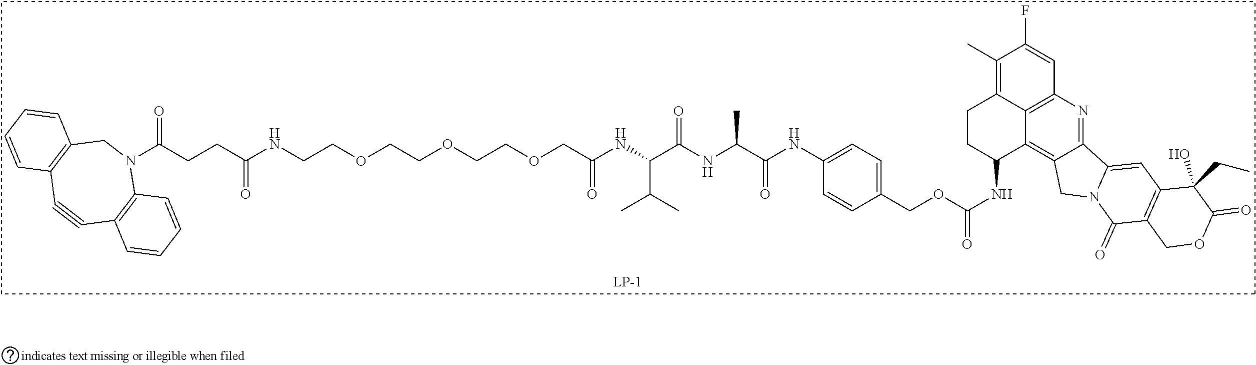

### i) (1-(9H-fluoren-9-yl)-3-oxo-2,7,10, 13-tetraoxa-4-azapentadecan-15-oyl)-L-valyl-L-alanine (Compound 34)

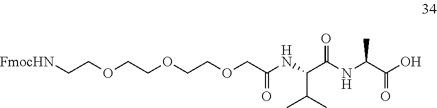

Compound 34 was synthesized using standard solid-phase Fmoc chemistry.
a) Resin loading: CH₂Cl₂ (200 mL) was added to 2-chlorotrityl chloride resin (6.0 mmol, 1.00 equiv.), followed by the addition of Fmoc-Ala-OH (1.0 equiv.) and DIPEA (6.0 equiv.), the mixture was agitated under N₂ atmosphere at 25 °C for 2 h. Thereafter methanol (9.5 mL) was added to the resin and the agitation continued for 30 min. The resin was then filtered and washed with DMF (300 ml×3).
b) Deprotection: 20% piperidine in DMF (200 mL) was added to the resin and agitated under N₂ atmosphere at 25 °C for 30 min. The resin was washed with DMF (200 mL×5) and filtered to get the resin with reactive amine group.
c) Coupling: A solution of HBTU (2.85 equiv.), and Fmoc-Val-OH (3.0 equiv.) in DMF (200 mL) was added to the resin followed by the addition of DIEPA (6.0 equiv.). The mixture was agitated under N₂ atmosphere at 25 °C for 30 min. The resin was then washed with DMF (200 mL×3). 15
d) Repeat step b to deprotect Fmoc group. Treat the resulting resin with Fmoc-N-amido-PEG3-acid (2.0 equiv.), HATU (1.9 equiv.) and DIPEA (4.0 equiv.) in DMF. The mixture was agitated under N₂ atmosphere at 25 °C for 30 min. The resulting resin was washed with DMF (200 mL×3).
e) Peptide cleavage and purification: The resin was washed with methanol (200 mL×3) and dried under vacuum. The dried resin was treated with the cleavage buffer consisting of 20% HFIP in CH₂Cl₂ and stirred for 30 min and filtered. Concentration of the filtrate under reduced pressure furnished the crude compound 34 (1.71 g) which was taken forward without further purification.

### ii) (9H-fluoren-9-yl) methyl ((2S,5S)-1-((4-(hydroxymethyl)phenyl)amino)-5-isopropyl-2-methyl-1,4,7-trioxo-9, 12, 15-trioxa-3,6-diazaheptadecan-17-yl) carbamate (Compound 36)

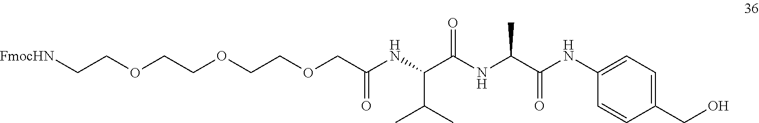

To a solution of compound 34 (1.60 g, 2.67 mmol, 1.0 equiv.) in CH₂Cl₂ (16.0 mL) was added compound 35 (657 mg, 5.34 mmol, 2.0 equiv.), and EEDQ (1.32 g, 5.34 mmol, 2.0 equiv.). The reaction mixture was stirred at 25 °C for 12 h. LCMS analysis showed the starting material 34 was consumed completely, and desired product mass was detected. The reaction mixture was added to 160 mL isopropyl ether, then centrifuged to afford the crude compound 36 (2.00 g) as a yellow oil. The crude product was used for the subsequent reaction without further purification. MS (ESI): [M+Na]⁺: 727.4.

### iii) (9H-fluoren-9-yl)methyl ((2S,5S)-5-isopropyl-2-methyl-1-((4-((((4-nitrophenoxy)carbonyl)oxy)methyl)phenyl)amino)-1,4,7-trioxo-9,12, 15-trioxa-3,6-diazaheptadecan-17-yl)carbamate (Compound 38)

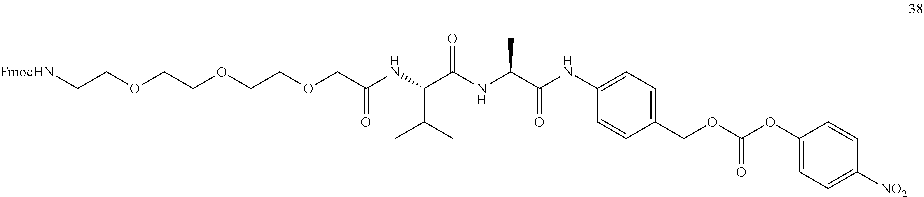

To a solution of compound 36 (2.00 g, 2.6 mmol, 1.0 equiv.), and compound 37 (1.58 g, 5.2 mmol, 2.0 equiv.) in DMF (20 mL) was added DIEPA (671 mg, 5.20 mmol, 905 μL, 2.0 equiv.). The mixture was stirred at 25 °C for 2 h. LCMS analysis showed 31% of desired product formation and 2% of the starting alcohol 36. The mixture was purified directly by purified by prep-HPLC (TFA condition) and the solvent was removed to furnish compound 38 (680 mg, 765 μmol, 29.4% yield, 97.9% purity) as a yellow solid. MS (ESI): [M+Na]⁺: 870.4.

### iv) (9H-fluoren-9-yl)methyl ((2S,5S)-1-((4-(((((1R,9S)-9-ethyl-5-fluoro-9-hydroxy-4-methyl-10,13-dioxo-2,3,9,10,13,15-hexahydro-1H, 12H-benzo[de]pyrano[3′,4′: 6,7]indolizino[1,2-b]quinolin-1-yl)carbamoyl)oxy)methyl)phenyl)amino)-5-isopropyl-2-methyl-1,4,7-trioxo-9,12, 15-trioxa-3,6-diazaheptadecan-17-yl)carbamate (Compound 39)

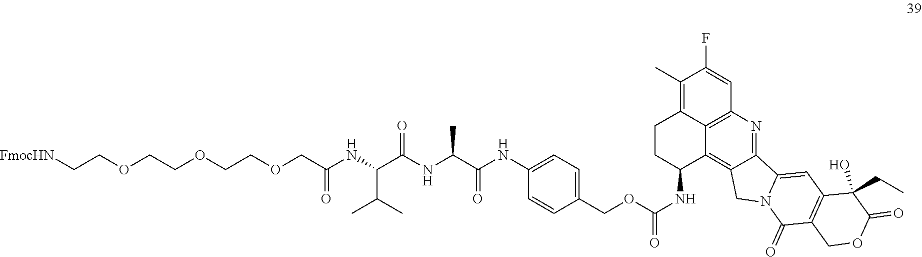

To a solution of compound 38 (200 mg, 225 μmol, 1.0 equiv.), and Exatecan (119 mg, 225 μmol, 1.00 equiv.) in DMF (3.40 mL) was added HOBt (33.4 mg, 247 μmol, 1.1 equiv.) and DIPEA (58 mg, 450 μmol, 78.4 L, 2.0 equiv.). The mixture was stirred at 25 °C for 2 h. LCMS analysis showed compound 38 was completely consumed and the desired product mass was identified. The mixture was purified directly by prep-HPLC (TFA condition) to afford compound 39 (180 mg, 148 μmol, 66% yield, 96.3% purity) as a yellow solid. MS (ESI): [M+H]⁺: 1167.7

### v) 4-((2,5S)-17-amino-5-isopropyl-2-methyl-4,7-dioxo-9,12, 15-trioxa-3,6-diazaheptadecanamido)benzyl ((1R,9S)-9-ethyl-5-fluoro-9-hydroxy-4-methyl-10,13-dioxo-2,3,9,10, 13, 15-hexahydro-1H, 12H-benzo[de]pyrano[3′,4′: 6,7]indolizino[1,2-b]quinolin-1-yl)carbamate (Compound 40)

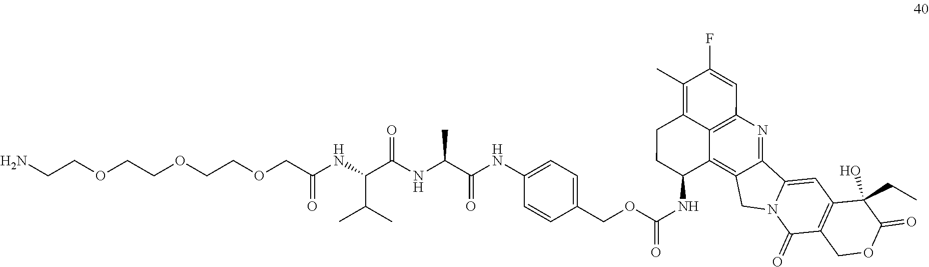 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)COCCOCCOCCN)C(C)C)cc1)CC3]

To a solution of compound 39 (180 mg, 148 μmol, 1.0 equiv.) in DMF (1.26 mL) was added triethylamine (392 mg, 3.88 mmol, 0.54 mL, 26 equiv.). The mixture was stirred at 25 °C for 2 h. LCMS analysis showed compound 39 was consumed completely, and desired product mass was detected. Compound 40 (140 mg, crude) was obtained as a brown liquid and used directly for the next step. MS (ESI): [M+H]⁺: 945.4

### vi) Synthesis of LP-1

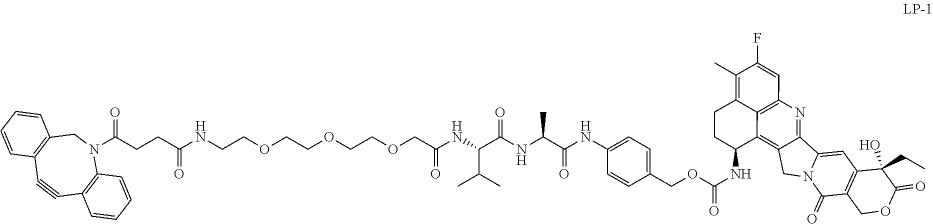 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)COCCOCCOCCNC(=O)CCC(=O)N2Cc4ccccc4C#Cc4ccccc42)C(C)C)cc1)CC3]

To a solution of compound 40 (140 mg, 148 μmol, 1.0 equiv.) in DMF (1.26 mL) was added triethylamine (392 mg, 3.9 mmol, 0.54 mL, 26.1 equiv.), and DBCO-OSu (59.6 mg, 148 μmol, 1.0 equiv.). The mixture was stirred at 25 °C for 1 h. LC-MS showed compound 40 was consumed completely, and desired mass was detected. The reaction mixture was added to 18.0 mL of isopropyl ether, and the crude product was slowly precipitated out. Centrifuged to get the crude product and discarded the liquid supernatant. The residue was purified by prep-HPLC (TFA condition) to obtain LP-1 (61.0 mg, 47.5 μmol, 32.5% yield, 97.5% purity) as a yellow solid. MS (ESI): [M+Na]⁺: 1253.6; ¹H NMR (400 MHz, DMSO-d₆) δ ppm 9.98 (s, 1H), 8.36 (d, J=6.80 Hz, 1H), 8.04-8.06 (m, 1H), 7.71-7.78 (m, 2H), 7.64-7.66 (m, 1H), 7.58 (br d, J=8.8 Hz, 3H), 7.41-7.47 (m, 4H), 7.25-7.37 (m, 6H), 5.44 (s, 2H), 5.28 (br s, 2H), 5.07 (s, 2H), 5.00 (br d, J=13.88 Hz, 1H), 4.38 (br t, J=6.94 Hz, 1H), 4.28 (dd, J=9.13, 6.63 Hz, 1H), 3.93 (s, 2H), 3.43-3.61 (m, 12H), 3.27 (br t, J=6.00 Hz, 4H), 3.03-3.11 (m, 2H), 2.37 (s, 3H), 2.13-2.26 (m, 3H), 1.93-2.03 (m, 2H), 1.81-1.92 (m, 2H), 1.70-1.80 (m, 1H), 1.30 (d, J=7.00 Hz, 3H), 0.84-0.91 (m, 6H), 0.81 (br d, J=6.75 Hz, 3H).

### Example 3—Synthesis of Linker-Payload Molecule-2 (LP-2)

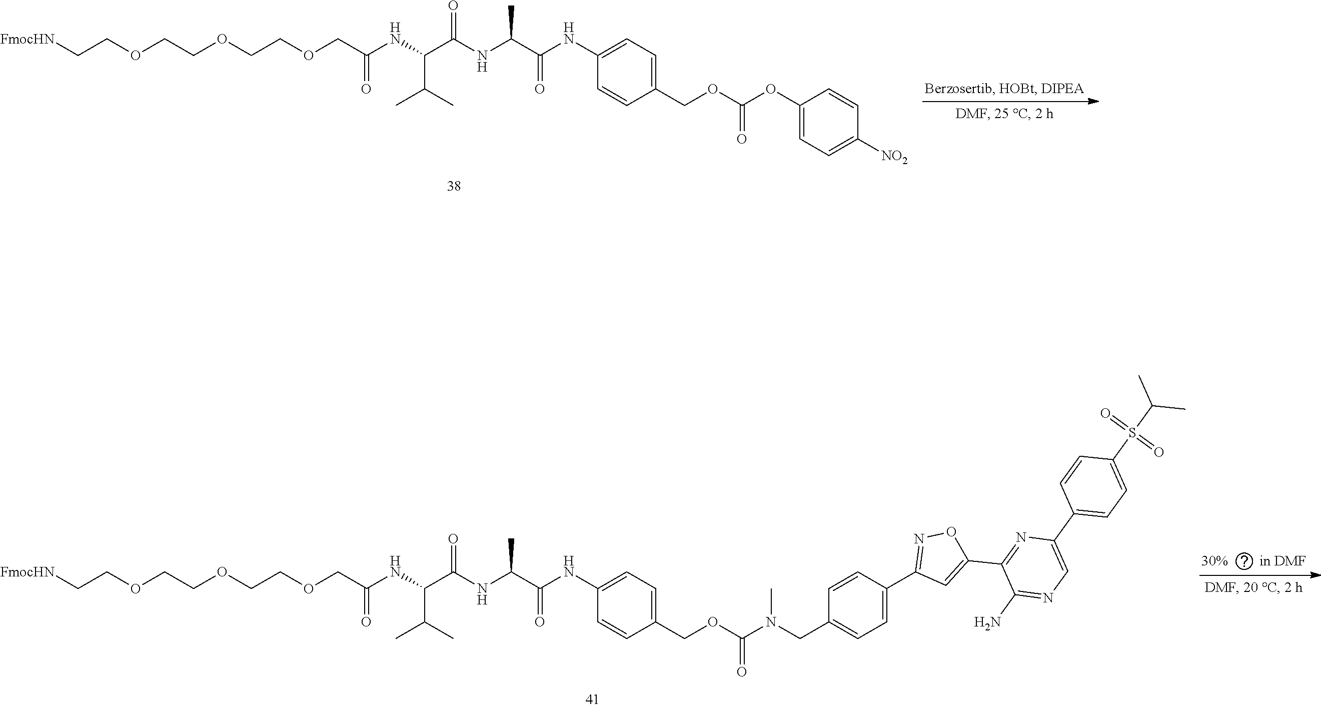

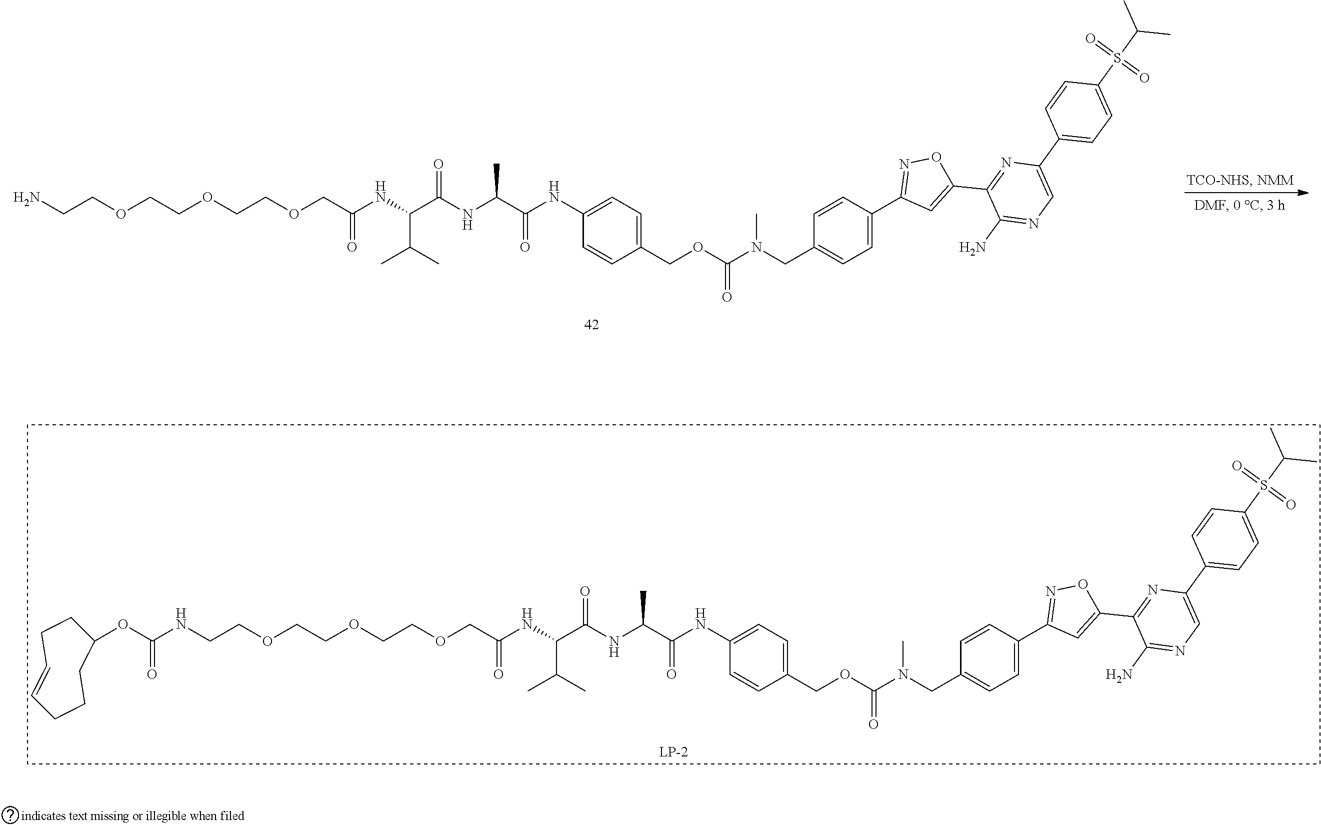 [SMILES: CC(C)[C@H](NC(=O)COCCOCCOCCN)C(=O)N[C@@H](C)C(=O)Nc1ccc(COC(=O)N(C)Cc2ccc(-c3cc(-c4nc(-c5ccc(S(=O)(=O)C(C)C)cc5)cnc4N)on3)cc2)cc1 ; CC(C)[C@H](NC(=O)COCCOCCOCCNC(=O)OC1CC/C=C/CCC1)C(=O)N[C@@H](C)C(=O)Nc1ccc(COC(=O)N(C)Cc2ccc(-c3cc(-c4nc(-c5ccc(S(=O)(=O)C(C)C)cc5)cnc4N)on3)cc2)cc1]

### i) 4-((17S,20S)-1-(9H-fluoren-9-yl)-17-isopropyl-20-methyl-3,15,18-trioxo-2,7,10,13-tetraoxa-4,16,19-triazahenicosan-21-amido)benzyl (4-(5-(3-amino-6-(4-(isopropylsulfonyl)phenyl)pyrazin-2-yl)isoxazol-3-yl)benzyl)(methyl)carbamate (Compound 41)

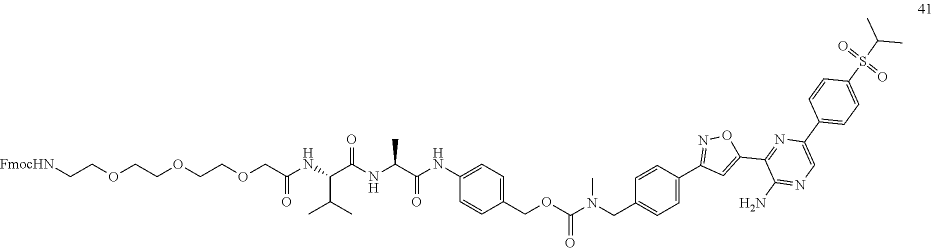

To a solution of compound 38 (150 mg, 168 μmol, 1.0 equiv.), and berzosertib (86 mg, 185 μmol, 1.1 equiv.) in DMF (1.5 mL) was added HOBt (25.0 mg, 185 μmol, 1.10 eq) and DIPEA (43.6 mg, 337 μmol, 58.8 μL, 2.00 eq). The mixture was stirred at 25 °C for 2 h. LCMS analysis showed compound 38 was consumed completely and the desired mass was detected. The mixture was purified directly by prep-HPLC (TFA condition) to afford compound 41 (150 mg, 121 μmol, 72.2% yield, 97.1% purity) as a yellow solid. MS (ESI): [M+H]⁺: 1194.6

### ii) 4-((2S,5S)-17-amino-5-isopropyl-2-methyl-4,7-dioxo-9,12, 15-trioxa-3,6-diazaheptadecanamido)benzyl (4-(5-(3-amino-6-(4-(isopropylsulfonyl)phenyl)pyrazin-2-yl)isoxazol-3-yl)benzyl)(methyl) carbamate (Compound 42)

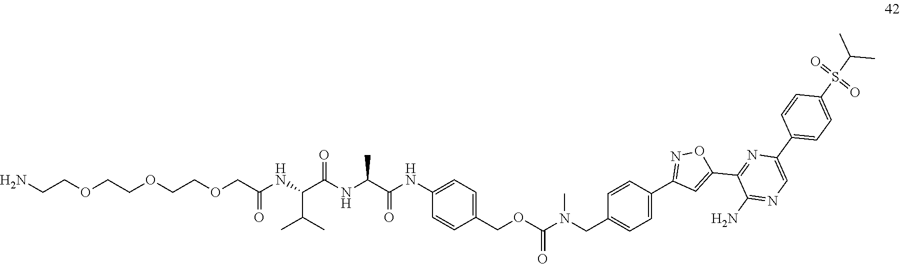 [SMILES: CC(C)[C@H](NC(=O)COCCOCCOCCN)C(=O)N[C@@H](C)C(=O)Nc1ccc(COC(=O)N(C)Cc2ccc(-c3cc(-c4nc(-c5ccc(S(=O)(=O)C(C)C)cc5)cnc4N)on3)cc2)cc1]

To a solution of compound 41 (150 mg, 121 μmol, 1.0 equiv.) in DMF (1.0 mL) was added triethylamine (327 mg, 3.23 mmol, 450 μL, 26.5 equiv.). The mixture was stirred at 25 °C for 2 h. LCMS analysis showed compound 41 was consumed completely and the desired product mass was observed. The reaction mixture was added to 15.0 mL isopropyl ether and centrifuged to get the crude compound 42 (200 mg, crude) as a yellow oil. MS (ESI): [M+H]⁺: 972.5

### iii) LP-2

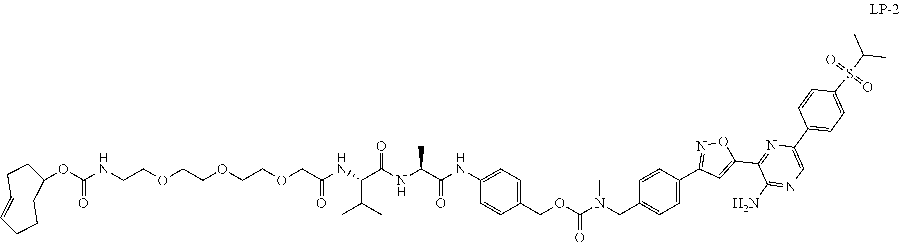 [SMILES: CC(C)[C@H](NC(=O)COCCOCCOCCNC(=O)OC1CC/C=C/CCC1)C(=O)N[C@@H](C)C(=O)Nc1ccc(COC(=O)N(C)Cc2ccc(-c3cc(-c4nc(-c5ccc(S(=O)(=O)C(C)C)cc5)cnc4N)on3)cc2)cc1]

To a solution of compound 42 (190 mg, 169 μmol, 1.0 equiv.), and TCO-NHS (45.2 mg, 169 μmol, 1.0 equiv.) in DMF (1.5 mL) was added NMM (17.1 mg, 169 μmol, 18.6 μL, 1.00 equiv.). The mixture was stirred at 0 °C for 3 h. LCMS showed compound 42 was consumed completely. The resultant reaction mixture was purified directly by prep-HPLC (neutral condition) to afford compound LP-2 (55.0 mg, 45.0 μmol, 27.6% yield, 95.6% purity) as a light-yellow solid. MS (ESI): [M+Na]⁺: 1146.6; ¹H NMR (400 MHz, DMSO-d₆): δ ppm 9.99 (s, 1H), 8.94 (s, 1H), 8.38 (d, J=8.50 Hz, 3H), 7.96-8.03 (m, 2H), 7.93 (d, J=8.63 Hz, 2H), 7.77 (s, 1H), 7.53-7.63 (m, 2H), 7.25-7.46 (m, 5H), 7.19 (br s, 2H), 6.86-6.93 (m, 1H), 5.49-5.60 (m, 1H), 5.36-5.46 (m, 1H), 5.07 (br s, 2H), 4.53 (s, 2H), 4.35-4.43 (m, 1H), 4.26-4.32 (m, 1H), 4.14-4.23 (m, 1H), 3.93 (s, 2H), 3.43-3.63 (m, 10H), 2.98-3.11 (m, 2H), 2.88 (s, 3H), 2.17-2.30 (m, 3H), 1.93-2.05 (m, 1H), 1.75-1.92 (m, 4H), 1.44-1.68 (m, 3H), 1.26-1.35 (m, 3H), 1.19 (d, J=6.75 Hz, 6H), 0.77-0.93 (m, 6H).

### Example 4—Effects of DDR Inhibitors and TOP1 Inhibitors on the DNA Damage Response Signalling Pathway

The effects of DDR inhibitor and TOP1 inhibitors, alone and in combination, on DNA damage response (DDR) signaling was analysed in vitro.

JIMT1 (TOP1 inhibitor resistant, breast cancer cell line) or HCT-116 cells were seeded in 10 cm plates at 1 and 0.8×10⁶ cells per/well respectively, using complete growth media (RPMI and McCoy respectively). Following overnight incubation, cells were serum starved using serum free media for at least 18 h. Cells were then treated with varying concentrations of berzosertib and/or exatecan for 24 h and 72 h, using complete growth media. Following incubation, cells were harvested using accutase, and lysed in IP lysis buffer (ThermoScientific, cat no. 87788) according to manufacturer's protocol and quantified using BCA assay (ThermoScientific, cat no. 23225). 50 μg of each protein sample was fractionated by SDS-PAGE and transferred to PVDF membrane using Bio-Rad semi-dry transfer cell. Membranes were blocked with 5% milk in TBS and immunoblotted with the indicated primary antibodies: PATR S428 (CS2853) 1:1000, overnight incubation at 4 °C; pCHK1 S317 (CS2344) 1:1000, overnight incubation at 4 °C; CHK1 (CS2360) 1:1000, overnight incubation at 4 °C; pH2AX S139 (CS9718) 1:1000, overnight incubation at 4 °C; β-Actin (8H10D10) (CS3700S) 1:1000, overnight incubation at 4 °C. Following incubation, blots were washed thrice with TBST and incubated with either anti-rabbit HRP (CS7074S) 1:1000 or anti-mouse HRP (CS7076S) secondary antibody used at 1:1000 dilution, and blots were incubated for 1 hr at room temperature. Blots were visualized with Bio-Rad Clarity Western ECL substrate (BioRad, cat no. 170-5061) and a ChemiDoc Imaging System (BioRad).

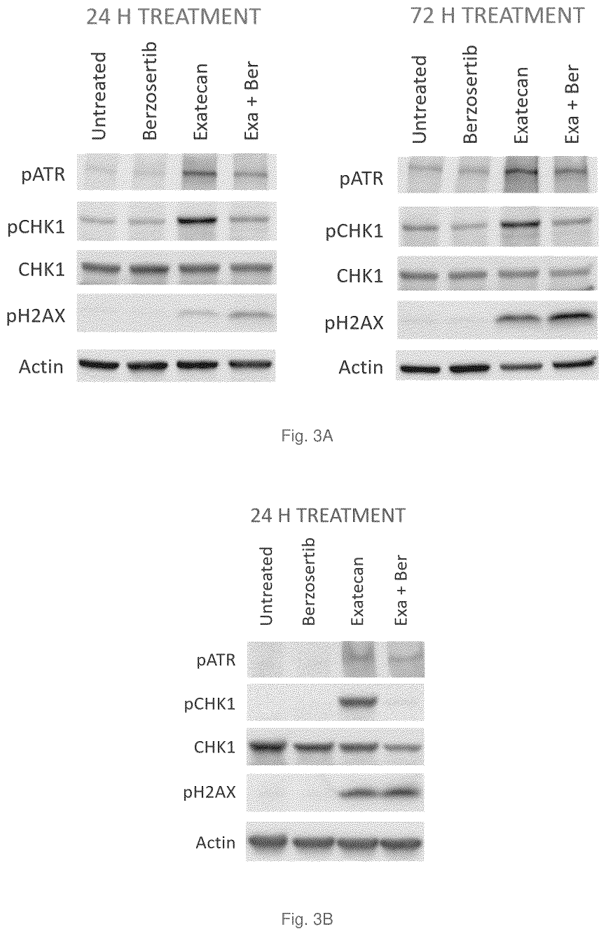

The exatecan and berzosertib combination significantly inhibits DNA damage response signaling (FIG. 3A, FIG. 3B). Exatecan alone induces pATR and pCHK1 of the DDR signaling pathway as well as DNA damage marker pH2AX. Addition of berzosertib to exatecan blocks DDR signaling pathway as seen by the decrease in pATR and pCHK1 and further increase in pH2AX indicating enhanced DNA damage.

### Example 5—Analysis of Toxicology of DDR Inhibitor+TOP1 Inhibitor Free Payload Combination

A free payload combination toxicology study was performed to assess synergistic or overlapping toxicities for the payloads in combination. The toxicological effects of exatecan and berzosertib in combination were investigated in female Sprague-Dawley rats aged 6 to 8 weeks old.

The following agents and concentrations were used:
Vehicle (20% DMSO (MedChemExpress, #HY-Y0320), 45% PEG300 (Selleckchem, #S6704), 5% Tween 20 (MedChemExpress, #HY-Y1891), 35% PBS (Sigma, #D8537-500 ML))
Exatecan (MedChemExpress, #HY-13631); Concentration: 1 mg/kg, 3 mg/kg, 10 mg/kg, 30 mg/kg
Berzosertib (Selleckchem, #S7102); Concentration: 35 mg/kg
Exatecan and berzosertib combination; Concentration: 1 mg/kg Exa+35 mg/kg Ber, 3 mg/kg Exa +35 mg/kg Ber, 10 mg/kg Exa+35 mg/kg Ber, 30 mg/kg Exa+35 mg/kg Ber

Mice were administered with the agents intravenously on Day 1 and Day 8.

Mortality observations and clinical observations were taken for all animals once daily. Body weight was assessed for all animals once pre-study and weekly thereafter.

Clinical chemistry and hematology assessments were performed on Day 2, Day 7, Day 9 and Day 11. 50 μl of fresh blood for hematology was collected via cheek bleed using EDTA as anti-coagulant and analysed using Vetscan HM5 analyser (Zoetis). 100 μl of fresh blood for biochemistry was collected via cheek bleed using lithium heparin as anti-coagulant and analysed using Vetscan VS2 chemistry analyser (Abaxis) using VetScan Comprehensive Diagnostic Profile reagent rotor (ZOETIS #10023219). Data were analyzed using Excel and GraphPad PRISM.

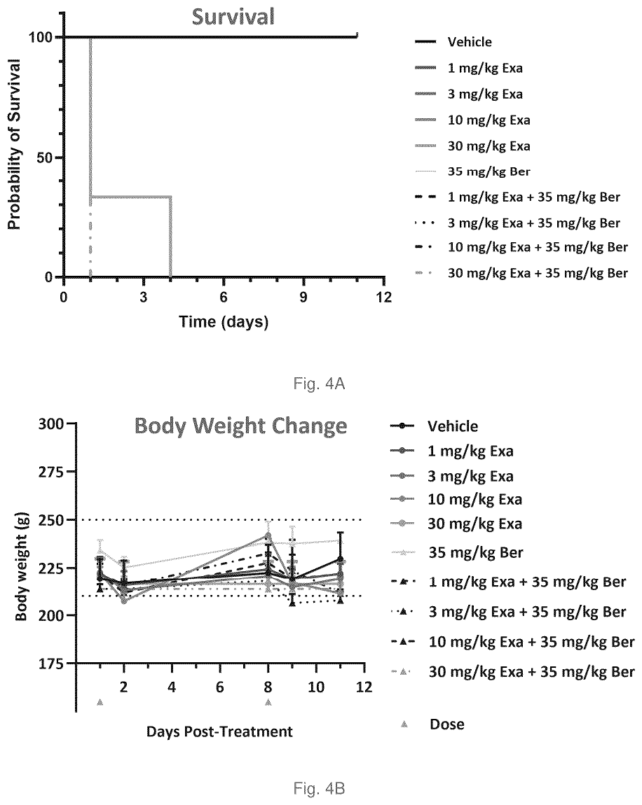

The results show that exatecan alone or in combination with berzosertib was well tolerated up to 10 mg/kg, and that berzosertib alone or in combination with exatecan (up to 10 mg/kg), was well tolerated at 35 mg/kg. No animal deaths were observed at 35 mg/kg berzosertib and 1, 3, and 10 mg/kg exatecan doses, alone or in combination (FIG. 4A). No significant changes in body weight were observed for single or combination payload arms relative to vehicle (FIG. 4B).

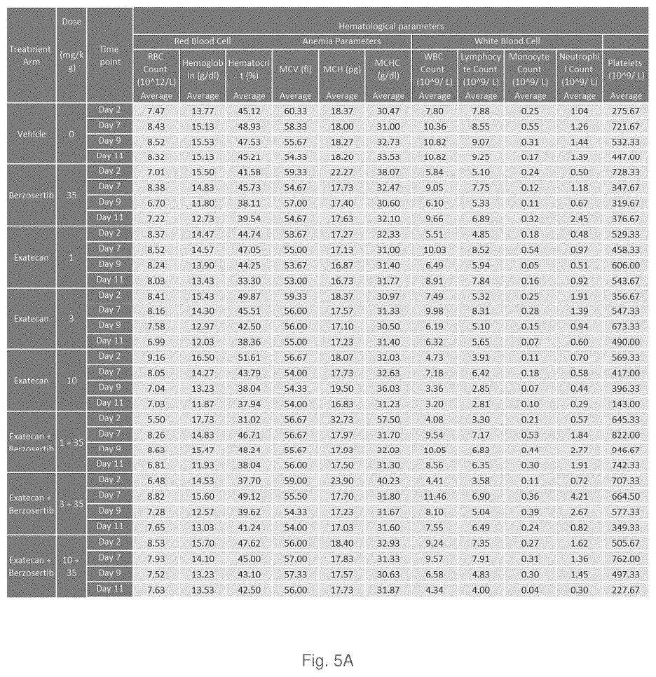

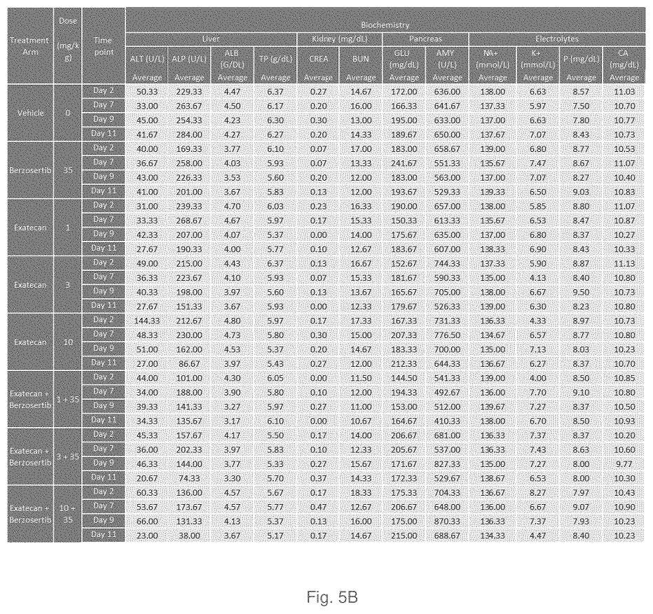

At the tolerated doses, there were no significant differences in toxicity observed between the combination and single payload arms. The payload combination did not result in any additional/unexpected toxicity or exacerbation of toxicity signals over single payloads. No significant change in body weight were observed at tolerated doses for exatecan and/or berzosertib. At tolerated doses, all differences in hematology and clinical chemistry test results were either transient, negligible in magnitude, within the normal biological variation, and/or lacked a dose relationship (FIG. 5A, FIG. 5B).

The 10 mg/kg tolerated dose for exatecan provides a 5X safety margin relative to the total amount of exatecan delivered in an ADC format at the maximum dose routinely tested for ADCs in rodent toxicology studies. The 35 mg/kg tolerated dose for berzosertib provides a 15X safety margin relative to the total amount of berzosertib delivered in an ADC format at the maximum dose routinely tested for ADCs rodent toxicology studies.

1-18. (canceled)

19. An antigen-binding molecule that binds to TROP2, comprising:
i. a TROP2-binding moiety;
ii. a branched linker moiety;
iii. a DNA topoisomerase I (TOP1) inhibitor moiety; and
iv. an Ataxia-Telangiectasia and RAD3-Related (ATR) inhibitor moiety,
wherein the TOP1 inhibitor moiety and the ATR inhibitor moiety are connected to the TROP2-binding moiety through the branched linker moiety.

20. The antigen-binding molecule of claim 19, wherein the ATR inhibitor moiety is linked to a click group selected from:
(i) an azide group;
(ii) an alkyne;
(iii) a tetrazine or tetrazine derivative group;
(iv) a cyclooctyne, cyclooctyne derivative, or cyclooctyne analogue group; and
(v) a strained alkene,
wherein the click group is conjugated to a corresponding click group in the branched linker moiety.

21. The antigen-binding molecule of claim 19, wherein the TOP1 inhibitor moiety is linked to a click group selected from:
(i) an azide group;
(ii) an alkyne;
(iii) a tetrazine or tetrazine derivative group;
(iv) a cyclooctyne, cyclooctyne derivative, or cyclooctyne analogue group; and
(v) a strained alkene,
wherein the click group is conjugated to a corresponding click group in the branched linker moiety.

22. The antigen-binding molecule of claim 19, wherein the branched linker moiety comprises an amino group conjugated to a glutamine of the TROP2-binding moiety.

23. The antigen-binding molecule of claim 19, wherein the ATR inhibitor moiety is or comprises, M6620 (berzosertib), M4344 (VX-803), AZD6738 (ceralasertib), BAY1895344 (elimusertib), RP3500 (camonsertib), ATRN119, ART380, IMP9064, HRS2398, M1774, SC0245, LF0397, or NU6027.

24. The antigen-binding molecule of claim 19, wherein the TOP1 inhibitor moiety is, or comprises, camptothecin or a derivative thereof, exatecan, exatecan mesylate (DX-8951f), N-glycyl-exatecan, SN-38, DXd (1), DXd (2), irinotecan, etirinotecan, FL118, topotecan, gimatecan, belotecan, deruxtecan, rubitecan, lurtotecan, diflomotecan, karenitecan, silatecan, namitecan, elomotecan, DRF-1042, delimotecan, NSC606985, chimmitecan, ZBH-1205, Genz-644282, non-CPT1, indotecan, indimitecan, AZ14170132, SHR9265, Ed-04, KL610023, A1.9, ZD06519, P1003, P1021, VIP126, ZBH-01, or LMP-744.

25. The antigen-binding molecule of claim 19, wherein the TOP1 inhibitor moiety is, or comprises exatecan, belotecan, SN-38, or DXd.

26. The antigen-binding molecule of claim 19, wherein the TOP1 inhibitor moiety is, or comprises exatecan, and the ATR inhibitor moiety is, or comprises berzosertib.

27. The antigen-binding molecule of claim 19, wherein the TOP1 inhibitor moiety is

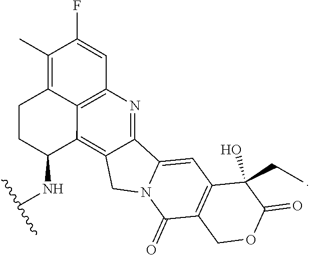 [SMILES: *N[C@H]1CCc2c(C)c(F)cc3nc4c(c1c23)Cn1c-4cc2c(c1=O)COC(=O)[C@]2(O)CC]

28. The antigen-binding molecule of claim 19, wherein the ATR inhibitor moiety is

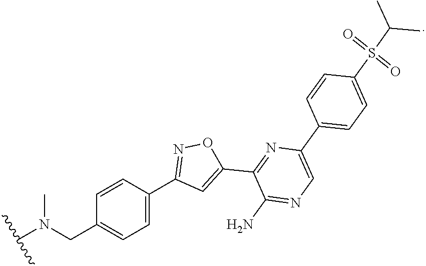 [SMILES: *N(C)Cc1ccc(-c2cc(-c3nc(-c4ccc(S(=O)(=O)C(C)C)cc4)cnc3N)on2)cc1]

29. The antigen-binding molecule of claim 21, wherein the TOP1 inhibitor moiety linked to a click group is:

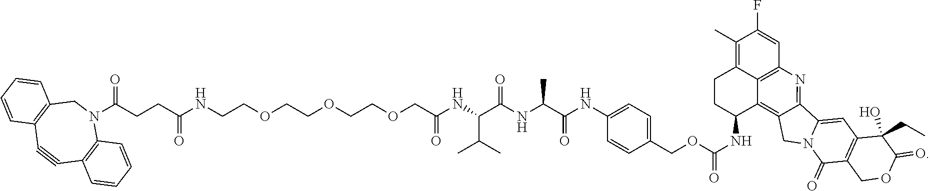 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)COCCOCCOCCNC(=O)CCC(=O)N2Cc4ccccc4C#Cc4ccccc42)C(C)C)cc1)CC3]

30. The antigen-binding molecule of claim 20, wherein the ATR inhibitor linked to a click group is:

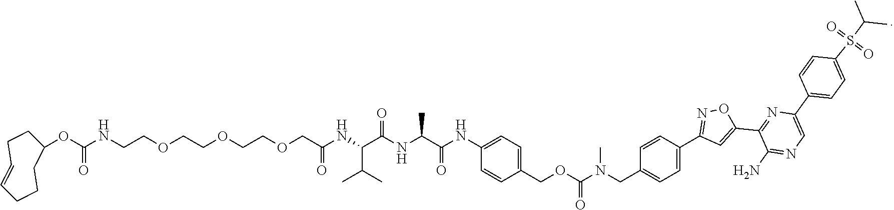 [SMILES: CC(C)[C@H](NC(=O)COCCOCCOCCNC(=O)OC1CC/C=C/CCC1)C(=O)N[C@@H](C)C(=O)Nc1ccc(COC(=O)N(C)Cc2ccc(-c3cc(-c4nc(-c5ccc(S(=O)(=O)C(C)C)cc5)cnc4N)on3)cc2)cc1]

31. The antigen-binding molecule of claim 19, wherein the TROP2-binding moiety comprises:
(i) a heavy chain variable (VH) region incorporating the following CDRs:
HC-CDR1 having the amino acid sequence of SEQ ID NO:13
HC-CDR2 having the amino acid sequence of SEQ ID NO:14
HC-CDR3 having the amino acid sequence of SEQ ID NO:15; and
(ii) a light chain variable (VL) region incorporating the following CDRs:
LC-CDR1 having the amino acid sequence of SEQ ID NO:21
LC-CDR2 having the amino acid sequence of SEQ ID NO:22
LC-CDR3 having the amino acid sequence of SEQ ID NO:23.

32. The antigen-binding molecule of claim 19, wherein the TROP2-binding moiety comprises:
a VH region comprising an amino acid sequence having at least 70% sequence identity to the amino acid sequence of SEQ ID NO:12; and
a VL region comprising an amino acid sequence having at least 70% sequence identity to the amino acid sequence of SEQ ID NO:20.

33. The antigen-binding molecule of claim 19, wherein the TROP2-binding moiety comprises:
a polypeptide comprising, or consisting of, an amino acid sequence having at least 70% sequence identity to the amino acid sequence of SEQ ID NO:10; and
a polypeptide comprising, or consisting of, an amino acid sequence having at least 70% sequence identity to the amino acid sequence of SEQ ID NO:11.

34. The antigen-binding molecule of claim 19, wherein the TROP2-binding moiety is hRS7 (Sacituzumab), MAAP-9001a (Datopotamab), PrE11, AR47A6.4.2, 7E6, 2EF, or Hu2G10, or an antigen binding fragment thereof.

35. The antigen-binding molecule of claim 19, wherein the TROP2-binding moiety comprises a substitution N297A.

36. The antigen-binding molecule of claim 19, wherein the branched linker moiety is conjugated to the TROP2-binding moiety via an enzymatic conjugation using a microbial transglutaminase.

37. A pharmaceutical composition comprising the antigen-binding molecule of claim 19, and a pharmaceutically acceptable carrier, diluent, excipient, or adjuvant.
